(19) United States
(12) Patent Application Publication
(10) Pub. No.: US 2006/0183221 A1
(43) Pub. Date: Aug. 17, 2006

(54) Method for neural differentiation of embryonic stem cells using protease passaging techniques

(76) Inventors: Thomas C. Schulz, Athens, GA (US); Brian G. Condle, Athens, GA (US); Allan J. Robins, Athens, GA (US)

Correspondence Address: Sutherland, Asbill & Brennan/Atta: Bill Warren, 999 Peachtree Street, NE, Atlanta, GA, 30309-3996, US

(21) Appl. No.: 10/551,603

(22) PCT Filed: Mar. 31, 2004
(86) PCT No.: PCT/US04/10121

§ 371 Date: Jan. 20, 2006

Related U.S. Application Data
(60) Provisional application No. 60/459,090, filed on Mar. 31, 2003.

(51) Int. Cl. C12N 5/08

(52) U.S. Cl. 435/366

(57) ABSTRACT

The present invention provides methods for human pluripotent cell culturing and for neural cell production. More particularly, the present invention provides culturing methods employing dissociating cell cultures to an essentially single cell culture, such as by employing antibody selection and bulk passaging treatments utilizing the subsequent application of Collagenase and trypsin. In certain embodiments, the cells are further treated with essentially serum free MEDII conditioned medium, proline, or minimal medium, and are optionally treated with amphiphilic lipid compounds for the generation of human neural cells from pluripotent human cells. In certain embodiments, the cells cultured using these methods have an abnormal karyotype.

### ACKNOWLEDGMENT OF FEDERAL RESEARCH SUPPORT

This invention was made, at least in part, with funding from the National Institutes of Health. Accordingly, the United States Government has certain rights in this invention.

### BACKGROUND OF THE INVENTION

1. Field of the Invention

This invention relates generally to mammalian stem cells and to differentiated or partially differentiated cells derived therefrom using methods for dissociating cells to an essentially single cell culture, such as by selecting cells with antibodies to pluripotent human cell markers, and protease passaging treatments. The invention also relates to mammalian stem cells and to differentiated or partially differentiated cells derived therefrom. The cell derived therefrom may be cultured with MEDII conditioned medium, proline, or a minimal medium, and optionally, may be cultured with amphiphilic lipid compounds, and preferably, with novel ceramide analogs of the β-hydroxyalkylamine type. The present invention also relates to methods of producing, differentiating and culturing the cells of the invention, and to uses thereof.

2. Background Art

Embryonic stem (ES) cells represent a powerful model system for the investigation of mechanisms underlying pluripotent cell biology and differentiation within the early embryo, as well as providing opportunities for genetic manipulation of mammals and resultant commercial, medical and agricultural applications. Furthermore, appropriate proliferation and differentiation of ES cells can be used to generate an unlimited source of cells suited to transplantation for treatment of diseases that result from cell damage or dysfunction. Other pluripotent cells and cell lines including early primitive ectoderm-like (EPL) cells as described in International Patent Application WO 99/53021, in vivo or in vitro derived ICM/epiblast, in vivo or in vitro derived primitive ectoderm, primordial germ cells (EG cells); teratocarcinoma cells (EC cells), and pluripotent cells derived by dedifferentiation, reprogramming or by nuclear transfer will share some or all of these properties and applications.

Human ES cells have been described in International Patent Application WO 96/23362, and in U.S. Pat. Nos. 5,843,780, and 6,200,806; and human EG cells have been described in International Patent Application WO 98/43679, and U.S. Pat. No. 6,245,566.

The ability to tightly control differentiation or form homogeneous populations of partially differentiated or terminally differentiated cells by differentiation in vitro of pluripotent cells has proved problematic. Most current approaches involve the formation of embryoid bodies from pluripotent cells in a manner that is not controlled and does not result in homogeneous populations. Mixed cell populations such as those in embryoid bodies of this type are generally unlikely to be suitable for therapeutic or commercial use.

Uncontrolled differentiation produces mixtures of pluripotent stem cells and partially differentiated stem/progenitor cells corresponding to various cell lineages. When these ES-derived cell mixtures are grafted into a recipient tissue the contaminating pluripotent stem cells proliferate and differentiate to form tumors, while the partially differentiated stem and progenitor cells can further differentiate to form a mixture of inappropriate and undesired cell types. It is well known from studies in animal models that tumors originating from contaminating pluripotent cells can cause catastrophic tissue damage and death. In addition, pluripotent cells contaminating a cell transplant can generate various inappropriate stem cell, progenitor cell and differentiated cell types in the donor without forming a tumor. These contaminating cell types can lead to the formation of inappropriate tissues within a cell transplant. These outcomes cannot be tolerated for clinical applications in humans. Therefore, uncontrolled ES cell differentiation makes the clinical use of ES-derived cells in human cell therapies impossible.

Selection procedures have been used to obtain cell populations enriched in neural cells from embryoid bodies. These include genetic modification of ES cells to allow selection of neural cells by antibiotic resistance (Li et al., 1998 Current Biol. 8:971-974), and manipulation of culture conditions to select for neural cells (Okabe et al., 1996 Mech. Dev. 59:89-102; and Tropepe et al., 2001 Neuron 30:65-78; O'Shea, 2002 Meth. in Mol. Biol. 198, 3-14). Previously, one research group has demonstrated efficient differentiation of mouse and primate ES cells to TH+ neurons following co-culture with the PA6 stromal cell line, but this technique is not likely to be useful for cell therapy applications as it introduces xenograft issues associated with exposure to non-human cell lines and removal of potential PA6 cell contamination in subsequent cultures (Kawasaki et al., 2000 Neuron 28, 3140; Kawasaki et al., 2002 Proc. Natl. Acad. Sci. USA, 99(3): 1580-1585). Furthermore, the PA6 differentiation procedure generated non-neural terminally differentiated cell types, such as retinal epithelial cells, reducing the usefulness of the cell cultures for cell therapy. In addition, McKay has demonstrated efficient differentiation of mouse ES cells to TH+ neurons, but this differentiation required over-expression of the Nurr-1 transcription factor in combination with exposure to Sonic Hedgehog and FGF8 (Kim et al., Nature 2002 418(6893):50-6). Furthermore, the McKay protocol involves a complex, five stage differentiation method for differentiation of mouse ES cells to neurons.

In all of these procedures, the differentiation of pluripotent cells in vitro does not involve biological molecules that direct differentiation in a controlled manner. Similarly, in experiments examining neural differentiation from human ES cells, there is no way to control the neural differentiation, and the methods merely allow for the passive development of neural cell types (see Zhang et al., 2001 Nature Biotech 19(12): 1129-1133, and Reubinoff et al., 2001 Nature Biotech 19(12); 1134-40). Hence homogeneous, synchronous populations of neural cells with unrestricted neural differentiation capability are not produced, restricting the ability to derive essentially homogeneous populations of partially differentiated or differentiated neural cells. Another research group differentiated human ES cell derived embryoid bodies in 20% serum containing medium for 4 days followed by plating and selection/expansion of neural cell types in medium containing B27 and N2 supplements (serum free), EGF, FGF-2, PDGF-AA, and IGF-1 (Carpenter et al., 2001 Exper. Neuro. 172, 383-397). Carpenter et al. showed that neural progenitors could be enriched from this culture system by cell sorting or immunopanning using antibodies directed against polysialated NCAM or the cell surface molecule recognized by the A2B5 monoclonal antibody.

Efficient neural differentiation of mouse embryonic stem cells in monolayer culture has recently been reported (Ying et al., 2003 Nature Biotechnology, 21:183-186). This previous study shows that adherent mouse ES cells can differentiate into neural cell types in a serum-free minimal medium. In contrast to the work described herein, the method described by Ying et al. produces neuronal cultures containing many GABAergic neurons and very few tyrosine hydroxylase expressing neurons. In addition the methods of Ying et al. are dependent on monolayer culture of the mouse ES cells.

Chemical inducers such as retinoic acid have also been used to form neural lineages from a variety of pluripotent cells including ES cells (Bain et al., 1995 Dev. Biol. 168:342-357, Strubing et al., 1995 Mech. Dev. 53, 275-287, Fraichard et al., 1995 J. Cell Sci. 108, 3181-3188, Schuldiner et al., 2001 Brain Res. 913, 201-205.). However, the route of retinoic acid-induced neural differentiation has not been well characterized, and the repertoire of neural cell types produced appears to be generally restricted to ventral somatic motor, branchiomotor or visceromotor neurons (Renoncourt et al., 1998 Mech. Dev. 79:185-197).

Manually passaged HESC colonies are typically comprised of tightly packed, multilayered, undifferentiated HESCs, and variable levels of cells undergoing early differentiation. When present, these differentiating cells are observed on the edges of HESC colonies and are considered to be an indicator that the maintenance of the undifferentiated state of the colony is beginning to be compromised. This is undesirable as the presence of differentiating cells is likely to have a negative influence on maintaining the undifferentiated state of the remaining HESC, as the differentiating cells can produce factors that influence cellular differentiation. Furthermore, the presence of differentiated cells is likely to add randomness to differentiation procedures due to the stochastic presence of these cells and the differentiation signals or factors that they produce. Due to the three dimensional nature of the manually passaged HESC cultures, differentiating cells are also likely to be present in regions of the colonies where they cannot be detected or distinguished morphologically. As shown by Henderson et al. (Stem Cells, 2002, 20:329-337), SSEA3 or SSEA1 magnetic bead based sorting of cells confirms the likelihood of different cell populations within a culture akin to manually passaged HESC cultures. There is therefore a need to develop methods to passage HESCs that result in more uniform populations of undifferentiated or partially undifferentiated cells, and that are not based on morphological distinctions.

Previous publications report the transplantation of ES-derived neural cells into the ventricles of the fetal or newborn rat or mouse brain without the formation of tumors (Brustle et al., 1997 PNAS 94:14809-14814, Zhang et al., 2001 Nature Biotech 19:1129-1133). Although some of the cells in these studies do integrate into the host brain, many of the cells in the transplants form neural tube-like structures within the lumen of the brain ventricle. Therefore, these previous studies do not lead to methods that can be readily applied to human cell therapy. Note that Reubinoff et al. (2001 Nature Biotech 19:1134) also injected ES-derived neural cells into the ventricles of newborn mice but did not report intraventricular masses of neural cells, omitting any mention of the presence or absence of such masses.

Neural stem cells and precursor cells have been derived from fetal brain and adult primary central nervous system tissue in a number of species, including rodent and human (e.g., see U.S. Pat. No. 5,753,506 (Johe), U.S. Pat. No. 5,766,948 (Gage), U.S. Pat. No. 5,589,376 (Anderson and Stemple), U.S. Pat. No. 5,851,832 (Weiss et al.), U.S. Pat. No. 5,958,767 (Snyder et al.) and U.S. Pat. No. 5,968,829 (Carpenter). However, each of these disclosures fails to describe a predominantly homogeneous population of neural stem cells able to differentiate into all neural cell types of the central and peripheral nervous systems, and/or essentially homogeneous populations of partially differentiated or terminally differentiated neural cells derived from neural stem cells by controlled differentiation. Furthermore, it is not clear whether cells derived from primary fetal or adult tissue can be expanded sufficiently to meet potential cell and gene therapy demands. Neural stem cells derived from fetal or adult brain are established and expanded after the cells have committed to the neural lineage and in some cases after the cells have committed to neural sublineages. Therefore, these cells do not provide the opportunity to manipulate the early differentiation processes that occur prior to neural commitment. Pluripotent stem cells provide access to these earliest stages of mammalian cellular differentiation opening additional options for cell expansion and directed development of the cells into desired lineages.

In summary, it has not been possible to control the differentiation of pluripotent cells in vitro, to provide homogeneous, synchronous populations of neural cells with unrestricted neural differentiation capacity. Similarly, methods have not been developed for the derivation of neural cells from pluripotent cells in a manner that parallels their formation during embryogenesis. In addition, current methods have relied upon the expression of foreign genes to drive neural differentiation of pluripotent stem cells (Kim et al, 2002 Nature 418:50-56). These limitations have restricted the ability to form essentially homogeneous, synchronous populations of partially differentiated and terminally differentiated neural cells in vitro, and have restricted their further development for therapeutic and commercial applications.

There is a need, therefore, to identify methods and compositions for the production of a population of cells enriched in neural stem cells and the products of their further differentiation, and in particular, human neural cells and their products.

### SUMMARY OF THE INVENTION

It is an object of the present invention to overcome, or at least alleviate, one or more of the difficulties or deficiencies associated with the prior art.

In that regard, the invention contemplates a human pluripotent cell culture, wherein the cells of the culture do not express SSEA1, express SSEA3, SSEA4, Oct4, Tra-1-60, Tra-1-80, and express nestin substantially uniformly. The present invention further provides a method of culturing a human pluripotent cell comprising dissociating a cell culture comprising human pluripotent cells to an essentially single cell culture. More specifically, the method of culturing a human pluripotent cell comprises: a) selecting a human pluripotent cell using an anti-SSEA4 antibody; and b) maintaining a culture of the cell by passaging the cell using a protease treatment, wherein the cells of the culture do not express SSEA1, express SSEA3, SSEA4, Oct4, Tra-1-60, Tra-1-80, and express nestin substantially uniformly. In a preferred embodiment the protease treatment comprises the sequential use of Collagenase and trypsin.

The invention further provides for a method of providing a human neural cell, comprising forming an embryoid body from a human pluripotent cell that had been dissociated to an essentially single cell culture. The invention contemplates that the human pluripotent cell was dissociated to an essentially single cell culture during at least one passage, and further contemplates that the dissociation preferably does not occur immediately prior to the formation of the embryoid body. The embryoid body may optionally be formed in the presence of MEDII conditioned medium, and/or a medium that contains proline, or a proline containing peptide, or in the presence of a minimal medium.

The invention further provides for a method of culturing a human pluripotent cell comprising, a) providing a human pluripotent cell; b) passaging the cell using a protease treatment comprising the sequential use of Collagenase and trypsin; c) dispersing the cell to an essentially single cell culture; and d) culturing the cell in the presence of a human feeder cell, in the presence of a conditioned medium, or in the presence of a minimal medium. In a further embodiment, the invention provides for a method of producing a human neural cell comprising, a) providing a human pluripotent cell; b) passaging the cell using a protease treatment comprising the sequential use of Collagenase and trypsin; c) dispersing the cell to an essentially single cell culture; d) culturing the cell in the presence of a human feeder cell, in the presence of a conditioned medium, or in the presence of a minimal medium; and e) forming an embryoid body comprising the essentially single cell culture by culturing the cell with an optionally essentially serum free medium. In a preferred embodiment, the essentially serum free medium is a MEDII conditioned medium or is a minimal medium.

The MEDII conditioned medium described herein can be preferably a Hep G2 conditioned medium that contains a bioactive component selected from the group consisting of a low molecular weight component; a biologically active fragment of any of the aforementioned proteins or components; and an analog of any of the aforementioned proteins or components. In a preferred embodiment, the bioactive component of the MEDII conditioned medium is proline, or a proline containing peptide. In one embodiment, the bioactive component of the MEDII conditioned medium is proline, preferably at a concentration of approximately 50 μM. The pluripotent human cell of the present invention can be selected from, but is not limited to, a human embryonic stem cell; a human ICM/epiblast cell; an EPL cell; a human primitive ectoderm cell; a human primordial germ cell; and a human EG cell.

In certain embodiments of the invention, the pluripotent cell culture of the invention that has been dissociated to an essentially single cell culture has an abnormal karyotype. In one embodiment, a majority of the cells have an abnormal karyotype. In further embodiments, the abnormal karyotype comprises a trisomy of at least one autosomal chromosome, wherein the autosomal chromosome is selected from the group consisting of chromosomes 1, 7, 8, 12, 14, and 17. In another embodiment, the abnormal karyotype comprises a trisomy of more than one autosomal chromosome, wherein at least one of the more than one autosomal chromosomes is selected from the group consisting of chromosomes 1, 7, 8, 12, 14, and 17. Preferably, the autosomal chromosome is chromosome 12 or 17. In another embodiment, the abnormal karyotype comprises an additional sex chromosome. In one embodiment, the karyotype comprises two X chromosomes and one Y chromosome. Combinations of the foregoing are also encompassed by the invention.

The invention further provides a composition comprising a culture of neural cells derived in vitro from a pluripotent human cell cultured with a composition comprising a ceramide compound. In preferred embodiments, these neural cells are capable of expressing one or more of the detectable markers for tyrosine hydroxylase (TH), vesicular monamine transporter (VMAT) dopamine transporter (DAT), and aromatic amino acid decarboxylase (AADC).

The invention further provides a method of treating a patient with a neural disease, comprising a step of administering to the patient a therapeutically effective amount of the neural cell or cell culture enriched in neural cells produced using the methods of the present invention.

The invention further provides for the human pluripotent cells and human neural cells produced using the methods of the invention.

### BRIEF DESCRIPTION OF THE DRAWINGS

FIGS. 1A-G show the chemical structure of ceramide, and novel structural analogs of ceramide (novel ceramide analogs or NCAs) synthesized by N-acylation of β-hydroxyalkylamines. A shows the chemical structure of N-acyl sphingosine (“ceramide”). B shows the chemical structure of N-(2-hydroxy-1-(hydroxymethyl)ethyl)-palmitoylamide (“S16”). C shows the chemical structure of N-(2-hydroxy-1-(hydroxymethyl)ethyl)-oleoylamide (“S18”). D shows the chemical structure of N,N-bis(2-hydroxyethyl)palmitoylamide (“B16”). E shows the chemical structure of N,N-bis(2-hydroxyethyl)oleoylamide (“B18”). F shows the chemical structure of N-tris(hydroxymethyl)methyl-palmitoylamide (“T16”). G shows the chemical structure of N-tris(hydroxymethyl)methyl-oleoylamide (“T18”).

FIG. 2 is a schematic showing the in vitro neural differentiation of mouse embryonic stem cells. Abbreviations: ES (embryonic stem cell); EB (embryoid: body); NP (neural progenitor cell); D (terminally differentiated cell); NEP (neuroepithelial precursor cell); GRP (glial restricted precursor cell); NRP (neuronal restricted precursor cell); LIF (leukemia inhibitory factor); DIV (days in vitro); FGF-2 (fibroblast growth factor 2); N2 (medium supplement N2); and Oct4, GFAP, and MAP-2, are markers for differentiation proteins.

FIG. 3 shows the levels of spontaneous and induced apoptosis in differentiating ES-J1 cells. During particular stages of in vitro neural differentiation, apoptosis was induced in ES-J1 cells by incubation for 20 hours with 35 μM C2-ceramide, 75 μM S18, or 100 μM S16. Apoptosis was determined by TUNEL staining. The levels of apoptosis in ceramide treated samples were compared to the levels in control samples that were not incubated with ceramide analogs. Each experiment was performed five times. The bars show the standard mean and deviation of % TUNEL positive cells that were counted in five areas of 200 cells in each experiment. Open bars, no ceramide analog treatment; black bars, ceramide analog treatment.

FIGS. 4A-J show the cell death of ES-J1 cells treated with the novel ceramide analog S18 during in vitro neural differentiation. FIGS. 4A and B show cell death in ES cells without and with S18 incubation, respectively. FIGS. 4C and D show cell death at the EB4 stage without and with S18 incubation, respectively. FIGS. 4E and F show cell death at the EB8 stage without and with S18 incubation, respectively. FIGS. 4G and H show cell death at the NP2 stage without and with S18 incubation, respectively. FIGS. 4I and J show cell death in differentiated neurons without and with S18 incubation, respectively. ES-J1 cells were differentiated in vitro following the protocol as described herein, and were subsequently incubated for 20 hours with 75 μM of the novel ceramide analog S18. Note the high degree of cell death that was induced at the EB8 (E, and F) and NP2 stages (G, and H), whereas differentiated neurons were unaffected by ceramide treatment (compare I to J). Note also that at the EB8 stage, a rim of cells surrounding the central embryoid body survived treatment with ceramide analogs. See FIG. 2 for an explanation of the differentiation stages.

FIGS. 5A, and B show Hoechst staining and nestin antibody staining of mouse EB8 cells after incubation with S18. Differentiating embryonic stem cells at stage EB8 were incubated for 24 hours with 80 μM of S18, and were then immunostained for nestin. Apoptosis was detected by intensive staining with Hoechst dye. Note that the center of the embryoid body (left side of A) stained strongly with Hoechst 33258 and indicates apoptotic cells, whereas the rim of non-apoptotic cells in the embryoid body stained intensively for nestin (B).

FIG. 6 shows a table summarizing double staining results for TUNEL and various marker proteins at the NP2 stage. TUNEL staining detects apoptotic cells, and the marker proteins indicate the stage of neural differentiation. The total number of cells staining for one specific antigen within a population of 200 cells was as follows: TUNEL, 65; PAR-4, 91; ceramide, 105; nestin, 113; and PCNA, 108. The table shows the number of cells that stained simultaneously for two antigens. Note that the TUNEL positive cells co-localized significantly less with nestin (8% of TUNEL positive cells were nestin positive cells while 57% of the total cell population was nestin positive cells) and that the TUNEL positive cells co-localized significantly more with PCNA (74% of TUNEL positive cells were PCNA positive cells while 54% of the total cell population was PCNA positive cells). A chi square analysis of these distributions showed that TUNEL positive cells were predominantly nestin negative and PCNA positive. The abbreviation “n.d.” indicates that a particular combination was not determined.

FIGS. 7A and B show that EB-derived stem cells treated with novel ceramide analogs of the serinol type do not form teratomas when injected into neonate mouse brains. Ten days after injection of the untreated ES cells (A) or treated ES cells (B), the brains were isolated for analysis. Massive teratoma formation was observed with untreated, control cells (A), while EB8-derived cells that have been treated with S18 did not show the formation of teratomas (B). The black India ink spot on the right side of the brain in panel B marks the injection channel.

FIGS. 8A-H show teratoma formation with untreated ES cells and tissue integration with S18-treated ES cells. EB8-derived stem cells were stained with a fluorescent marker dye (Vybrant diI) in order to track the migration and integration of the injected cells into the recipient's brain tissue. A and B show the injection site of untreated EB8-derived embryonic stem cells, while E and F show the injection site of S18 treated EB8-derived embryonic stem cells. C and D show the migration site of untreated EB8-derived embryonic stem cells, while G and H show the migration site of S18 treated EB8-derived embryonic stem cells. Brains injected with untreated cells show teratoma formation and displacement growth at the migration site (C and D). Only the center of the tumor is stained with Vybrant diI. In the periphery of the tumor, cells have undergone numerous cell divisions, resulting in dilution of the fluorescent dye and low levels of staining. Note the bright Vybrant diI staining of cells that have integrated into the recipient's brain tissue (G and H). This intensive staining indicates that the cells have undergone a limited number of cell divisions.

FIGS. 9A-D show expression of Oct4 protein in HESCs and serum free embryoid bodies. A shows high levels of Oct4 expression in a typical manually passaged HESC colony, with distinct nuclear expression in undifferentiated ES cells and no Oct4 in the unstained feeder layer surrounding the HESC colony. B shows a typical manually passaged HESC crater colony, showing high levers of Oct4 expression in the multilayered ring of undifferentiated cells surrounding the monolayer crater cells that express a low level of Oct4. Differentiating cells at the edge of the colony also express a low level of Oct4. C shows the expression of Oct4 in a seeded essentially serum free embryoid body, representative of what is seen when sfEBMs are derived from domed HESCs or monolayer crater cells. Regions of high level Oct4 expression persist and are indicative of residual nests of pluripotent cells maintained by local cell-cell signaling events. Neural rosettes in the same field are indicated as radially organized circles of nuclei by DAPI staining (D) and these neural precursor cells only express low levels of Oct4.

FIGS. 10A-E show the effect of S18 treatment on seeded sfEBMs. A shows a seeded essentially serum free embryoid body exhibiting neural rosettes within the core of the explant and other cell types that have proliferated away from the rosettes. B shows that a high proportion of cells within these cultures have been killed after 36 hours exposure to 6 μM S18. C shows that a high degree of cell death is apparent after 36 hours exposure to 8 μM S18. Neural rosettes appear to be unaffected and in many cases can be observed more clearly, as surrounding cell types have died. D is a 60× magnification of surviving neural rosette after 36 hours exposure to 8 μM S18. The rosette appears morphologically normal and the typical radial organization of cells and distinct boundary between healthy rosette cells and apoptotic surrounding cells can be observed. E shows that the dying cells are undergoing apoptosis. Apoptosis of dying cells is indicated by their fragmented nuclei when stained with DAPI. Morphologically normal nuclei of unaffected cells are present in the lower right corner.

FIGS. 11A and B demonstrate the purification of neural rosette material by exposure of sfEBMs in suspension to S18. A shows S18 resistant neural rosette material isolated from generally degenerating sfEBMs grown in suspension at 20×. B shows a 40× magnification of a different piece of S18 resistant neural rosette material.

FIGS. 12A and B show the ablation of residual pluripotent cells in sfEBM cultures exposed to S18. sfEBM cultures exposed to S18 in suspension, followed by seeding and immunocytochemistry do not exhibit any cells expressing high levels of Oct4. This demonstrated that residual nests of pluripotent cells did not survive S18 induced apoptosis.

FIGS. 13A-F show that neural rosette cells are unaffected by exposure to S18. FIGS. 13A, B, and C show the same field of seeded sfEBMs stained with anti-Oct4, anti-Map2 and anti-TH, respectively. Seeded rosette cells only express low levels of Oct4 (A) and mature neurons (Map2+; B) are either also resistant to S18 or are regenerated effectively from the rosette precursor cells. A proportion of the Map2+ cells are presumptively dopaminergic neurons as they express Tyrosine Hydroxylase (C), indicating that they are also resistant to S18 and/or the rosette precursor cells maintain their capacity to differentiate to dopaminergic neurons. D and E show 40× magnification of Map2 and TH positive neurons in the same field, respectively. F shows that neural rosettes were still proliferative after exposure to S18, as demonstrated by phosphoHistone H3 staining for mitotic cells (indicated as the intense white spots) within DAPI stained rosettes, shown as the paler staining radially organized structures.

FIGS. 14A-L show immunostaining of SSEA4 selected trypsin passaged cells. A and B show Oct4 and DAPI staining, respectively; C and D show SSEA1 and DAPI staining, respectively; E and F show SSEA3 and DAPI staining, respectively; G and H show SSEA4 and DAPI staining, respectively, I and J show Tra-1-60 and DAPI staining, respectively; and K and L show Tra-1-81 and DAPI staining respectively.

FIGS. 15A-D show Nestin expression in manually passaged and SSEA4 selected trypsin passaged cells. A and B show Nestin and DAPI staining of manually passaged HESCs, respectively. The edge of a HESC colony is shown, showing that multilayered cells toward the center of the colony do not exhibit nestin expression (indicated by the dot in the lower right corner), while nestin expressing cells encircle the colony (indicated by the arrowhead), which are in turn surrounded by an outer ring of differentiating nestin+cells (top left corner, indicated by the arrow). C and D show Nestin and DAPI staining of SSEA4 selected trypsin passaged HESCs, respectively. A substantially uniform distribution of nestin is exhibited.

FIGS. 16A-C show DAPI stained 3 μm plastic sections of manually and SSEA4 selected trypsin passaged cells. A shows sfEBMs derived from manually passaged crater cells, fixed at day 10. Well organized rosette regions can be observed. 50% of the sfEBMs consisted of neural rosette cells as determined by counting the nuclei. Arrows indicate regions of apoptosis/necrosis associated with non-rosette cell types. Note that rosette cells were viable even when located in the center of the sfEBM, unlike the non-rosette cell type(s) that were not viable when located more than approximately 5 cell widths from the edge of the EB. B shows sfEBMs derived from SSEA4 selected trypsin passaged HESCs, fixed at day 9 for sectioning. A very high proportion of the sfEBM is organized into small but densely packed rosettes. No apoptotic/necrotic regions were observed. C shows sfEBMs derived from SSEA4 selected trypsin passaged HESC, exposed from day 6-9 to 10 μM S18, and fixed at day 9 for sectioning. Nearly all cells exhibited a radial nuclear staining, with predominant organization into rosettes, indicating a highly enriched population of neural rosette cells.

FIGS. 17A-D show enhanced neural differentiation of SSEA4 selected trypsin passaged HESCs in response to MEDII. Serum free embryoid bodies were derived, exposed to 10 μM S18 from day 13 to day 17, seeded at day 18 and fixed for immunostaining at day 23. A and B show TH immunostaining and DAPI staining, respectively, of serum free embryoid bodies grown in FGF2. The proportion of TH+ cells and distribution of the network of the dopaminergic neural projections was considerably enhanced over what had previously been observed with serum free embryoid bodies derived from manually passaged HESCs. Up to 30-70% by area of the sfEBs contained TH+ neurons, as opposed to less than ˜20% for crater derived sfEBMs. Significant regions of the seeded embryoid bodies did not contain neurons. C and D show TH immunostaining and DAPI staining, respectively, of serum free embryoid bodies grown in FGF2/MEDII. A very high proportion of the culture, typically >90% of the area of a seeded sfEBM piece, consisted of TH+ neurons and the differentiation of these cells was enhanced, as they exhibited far more developed neural processes. Non-neural regions of the culture were significantly reduced. The proportion of neural rosettes appeared to be far greater in cultures exposed to MEDII.

FIGS. 18A-F show high efficiency dopaminergic differentiation. SSEA4 selected trypsin passaged HESCs were differentiated in response to MEDII to generate a very high proportion of TH+ neurons. Serum free embryoid bodies were derived, exposed to 10 μM S18 from day 13 to day 17, seeded at day 18 and fixed for immunostaining at day 23. A and B show βIII-Tubulin and DAPI staining, respectively, of a seeded sfEBM. The boxes mark the regions shown at increased magnification in C-F. C and E show an increased magnification of the TH immunostaining, and D and F show an increased magnification of the βIII-Tubulin immunostaining. A very high proportion, typically 90% or greater of the neurons express TH.

FIGS. 19A-B show a comparison of TH+ and Hoffman optics images of neural extensions in a region of a serum free embryoid body grown in 4 ng/ml FGF2. Serum free embryoid bodies were derived, exposed to 10 μM S18 from day 13 to day 17, seeded at day 18 and fixed for immunostaining at day 23. A very high proportion of neurons express TH.

FIGS. 20A-D show expression of TH and VMAT in sfEBM cultures. sfEBMs were derived, exposed to 10 μM S18 from day 13 to day 17, seeded at day 18 and fixed for immunostaining at day 23. A and C show VMAT expression at 40× and 20× magnification respectively. B and D show TH expression at 40× and 20× magnification respectively. TH+/VMAT−, TH−/VMAT+ and TH+/VMAT+ cells could be observed.

FIGS. 21A-B illustrate the dopamine release assay. A is a schematic representation of the purification, modification and competitive enzyme linked immunoassay. Dopamine (D) is released from cultured neurons by depolarization with KCl, D is then is purified with a cis-diol affinity resin and acylated to N-acyldopamine (D^{a}). D^{a} remains in suspension and is modified to N-acyl-3-Methoxytyamine (m), which competes with solid phase D for a limited number of anti-dopamine antibody binding sites. Free antigen and antibody are removed by washing, and antibody bound to solid phase D is detected with a secondary antibody-peroxidase conjugate. There is an inverse correlation between the amount of D in the samples and detected signal. The amount of D in the sample is established from a standard curve. B shows a determination of dopamine released from sfEBM samples, which had been derived, seeded to polyornithine/laminin coated slides at day 25 and cultured to day 30 prior to depolarization. The cultures released approximately 2650 pg/ml of dopamine into the depolarizing medium (dot and vertical line). This value was within the range of the standard curve (dots representing 0, 150, 600, 2400, 9600, 38400 pg/ml dopamine) and fell between two unknown control samples from the kit (arrows).

FIGS. 22A-D show sections of sfEBMs exposed to S18. The sfEBMs were derived from protease passaged HESCs exposed to 10 μM S18 from day 6 to 9 after derivation of the embryoid bodies. Sections were stained with DAPI to reveal rosette organization and nuclear morphology. A shows a section of an untreated sfEBM at day 9, while B-D show sections of sfEBMs at day 9 that were treated with S18 from days 6-9.

FIGS. 23A-E show the neural differentiation of SSEA4 selected bulk passaged cells cultured as serum free embryoid bodies in FGF2 and Proline. sfEBP were derived and cultured for 10 or 17 days, and seeded to polyornithine/laminin for 5 days. A, and B show seeded sfEBPs at day 15 stained with DAPI and anti-βIII-Tubulin, respectively, at 10× magnification. C, D, and E show seeded sfEBPs at day 22 stained with DAPI, anti-βIII-Tubulin and anti-TH, respectively, at 40× magnification.

FIG. 24 shows neural differentiation of SSEA4 selected bulk passaged cells cultured as serum free embryoid bodies in minimal medium without FGF2, MEDII or L-Proline. Serum free embryoid bodies were seeded at day 21, fixed at day 25 and immunostained with anti-βIII-Tubulin and imaged at 10× magnification.

FIGS. 25A-B show whole mount immunostaining and confocal analysis of 50 μM L-Proline sfEBP at day 27 after derivation. Different sfEBPs are shown in these images. A shows anti-βIII-Tubulin immunostaining, detected with an Alexa 488 labeled secondary antibody and 1 μm confocal section at 40× magnification. Complex networks of βIII-Tubulin positive neuronal extensions were detected. A non-staining neural rosette is indicated by the asterisk, and βIII-Tubulin positive cell bodies are indicated by arrowheads. B shows a DAPI stained sfEBP imaged at 1 μm sections by a 2-Photon laser confocal at 40× magnification. A large proportion of the sfEBP consists of the elongated, closely packed, radial, neural rosette nuclei. The two dashed ovals surround a rosette and indicate its central proliferative core, where mitotic figures are localized within rosettes.

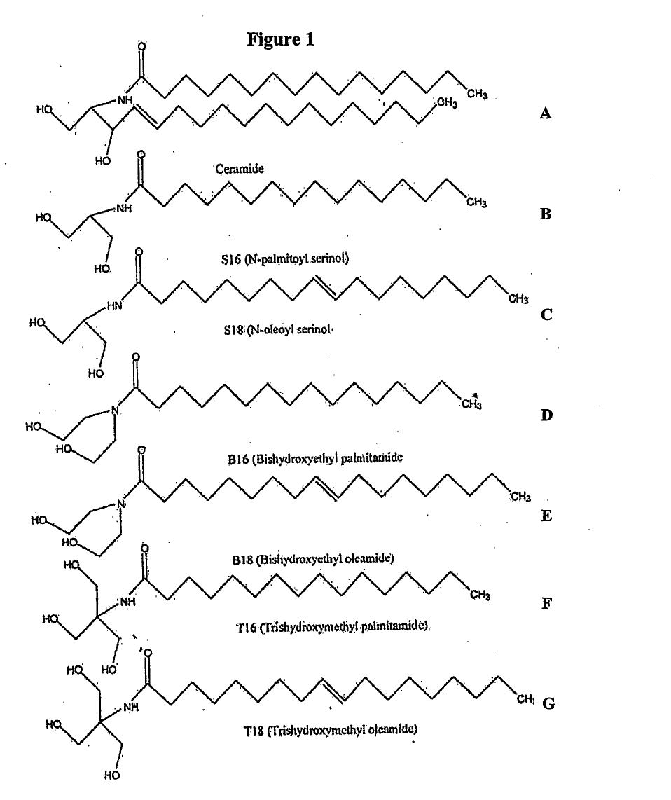

### DETAILED DESCRIPTION OF THE INVENTION

Applicant has demonstrated that culturing human cell populations comprising pluripotent human cells by dissociating the cells to an essentially single cell culture, such as by selecting the cells with an antibody directed to a pluripotent cell marker, and/or passaging the cells with a protease treatment results in the formation of a human pluripotent cell type that expresses cell markers characteristic of human embryonic stem cells, and also expresses nestin in a substantially uniform manner. When these cells are cultured with MEDII, they form neural cells with greater homogeneity than observed in a pluripotent human cell population that is not cultured with MEDII. When these cells are cultured with a minimal medium that optionally comprises proline, they form neural cells with greater homogeneity than observed in a pluripotent human cell population that is not cultured with minimal medium. This differentiation protocol has the capacity to be performed on a large scale, free of exposure to non-human cell types, to generate a high proportion of dopaminergic neurons, in the absence of residual pluripotent cells.

The differentiation approach using L-proline is the first example of high efficiency DA differentiation of mouse, monkey or human ES cells in a chemically defined medium, in the absence of exogenous neural or DA inducing factors such as FGF8/shh, the presence of inducing transgenes such as Nurr1, or the presence of stromal cell co-cultures. Given the requirement for HESC lines that have not been exposed to mouse feeder cells, the approach of the present invention represents the simplest and most viable approach when progressing toward clinical trials, enabling critical issues to be addressed, such as refinement of culture conditions, scaling and meeting FDA regulations.

In certain embodiments of the invention, the pluripotent cell culture that has been dissociated to an essentially single cell culture has an abnormal karyotype. In one embodiment, a majority of the cells have an abnormal karyotype. It is contemplated that greater than 50%, 55%, 60%, 65%, 70%, 75%, 80%, 85%, 90% or greater than 95% of metaphases examined will display an abnormal karyotype. In certain embodiments, the abnormal karyotype is evident after the cells have been dissociated to an essentially single cell culture for greater than 5, 6, 7, 8, 9, 10, 11, 12, 13, 14, 15, or 20 passages. Preferably, the abnormal karyotype is evident after the cell culture has been dissociated to a single cell culture for less than 10 passages. In one embodiment, the abnormal karyotype comprises a trisomy of at least one autosomal chromosome, wherein the autosomal chromosome is selected from the group consisting of chromosomes 1, 7, 8, 12, 14, and 17. In another embodiment, the abnormal karyotype comprises a trisomy of more than one autosomal chromosome, wherein at least one of the more than one autosomal chromosomes is selected from the group consisting of chromosomes 1, 7, 8, 12, 14, and 17. Preferably, the autosomal chromosome is chromosome 12 or 17. In another embodiment, the abnormal karyotype comprises an additional sex chromosome. In one embodiment, the karyotype comprises two X chromosomes and one Y chromosome. It is also contemplated that translocations of chromosomes may occur, and such translocations are encompassed within the term “abnormal karyotype.” Combinations of the foregoing chromosomal abnormalities are also encompassed by the invention.

In one embodiment, the pluripotent cell culture that has an abnormal karyotype is stable in culture. As used herein, the terms “stable” and “stabilize” refer to the differentiation state of a cell or cell line. When a cell or cell line is stable in culture, it will continue to proliferate over multiple passages in culture, and preferably indefinitely in culture; additionally, each cell in the culture is preferably of the same differentiation state, and when the cells divide, typically yield cells of the same cell type or yield cells of the same differentiation state. Preferably, a stabilized cell or cell line does not further differentiate or de-differentiate if the cell culture conditions are not altered, and the cells continue to be passaged and are not overgrown. It is preferred that the cells with an abnormal karyotype are stable in culture for greater than 5, 10, 15, 20, 25 or 30 passages.

In one embodiment, the neural cell produced by culturing the protease passaged and differentiated pluripotent human cell is therapeutically transplanted into the brain of a subject. The cell culture of the present invention form teratomas at a greatly reduced frequency than if the culture was not passaged using a protease treatment. In a preferred embodiment, the cell culture of the present invention does not induce the formation of teratomas at a significant rate.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed, Berlin: Springer-Verlag; in Current Protocols in Molecular Biology, F. M. Ausubel et al., eds., Current Protocols, a joint venture between Greene Publishing Associates, Inc. and John Wiley & Sons, Inc., (1998 Supplement); in Current Protocols in Cell Biology, J. S. Bonifacino et al., Eds., Current Protocols, John Wiley & Sons, Inc. (1999 Supplement); and in Current Protocols in Neuroscience, J. Crawley et al., Eds., Current Protocols, John Wiley & Sons, Inc. (1999 Supplement). It is to be understood that as used in the specification and in the claims, “a” or “an” can mean one or more, depending upon the context in which it is used. Thus, for example, reference to “a cell” can mean that at least one cell can be utilized.

The present invention particularly provides a human pluripotent cell culture, wherein the cells of the culture express SSEA3, SSEA4, Oct4, Tra-1-60, Tra-1-80, and express nestin substantially uniformly. In one embodiment, the cells do not express SSEA-1. In a further embodiment, the cells display an abnormal karyotype. In a preferred embodiment, the human pluripotent cell culture is at least partially differentiated towards a neural cell type. In another embodiment, the human pluripotent cell culture is reversibly partially differentiated towards a neural cell type. The invention further provides methods of producing a human pluripotent cell culture, wherein the cells of the culture express SSEA3, SSEA4, Oct4, Tra-1-60, Tra-1-80, and express nestin substantially uniformly, described in detail below. In one embodiment, the cells do not express SSEA-1.

The present invention further provides a method of culturing a human pluripotent cell, comprising the steps: a) selecting a human pluripotent cell using an anti-SSEA4 antibody; and b) maintaining a culture of the cell by passaging the cell using a protease treatment, wherein the cells of the culture express SSEA3, SSEA4, Oct4, Tra-1-60, Tra-1-80, and express nestin substantially uniformly. As used herein, the term “substantially uniformly” refers to the expression pattern of a cellular marker when a colony of cells is examined for expression of that marker. If there is “substantially uniform” expression of a marker, generally most of the cells of the colony express the marker. For example, if the center of an HESC colony does not express a marker, but the marker is expressed in most of the cells in the remainder of the colony, the marker is not expressed in a substantially uniform manner. Preferably, greater than 90% of the cells of a colony express the marker, more preferably, greater than 91%, 92%, 93%, 94%, 95%, 96%, 97%, 98%, of the cells of the colony express the marker, and still more preferably, greater than 99% of the cells of the colony express the marker.

In one embodiment, the protease treatment comprises the sequential use of Collagenase and trypsin. Preferably, Collagenase is used at a concentration of from approximately 0.1 mg/ml to approximately 10 mg/ml, more preferably from a concentration of from approximately 0.5 mg/ml to approximately 5 mg/ml, and most preferably at a concentration of from approximately 1 mg/ml to 2 mg/ml. The invention contemplates that Collagenase may be used for approximately 1 minute to 10 minutes, more preferably from approximately 2 minutes to 8 minutes, and most preferably for approximately 4 minutes to 6 minutes.

In another embodiment, trypsin is used at a concentration of from approximately 0.001% to 1%, more preferably at a concentration of from approximately 0.01% to 0.1%, and most preferably at a concentration of approximately 0.05%. The invention contemplates that trypsin may be used for approximately 1 second to 5 minutes, more preferably for approximately 5 seconds to 2 minutes, more preferably for approximately 10 seconds to 1 minute, and most preferably for approximately 30 seconds.

In a further preferred embodiment, Collagenase is used at a concentration of approximately 1 mg/ml for approximately 5 minutes, and trypsin is used at a concentration of approximately 0.05% for approximately 30 seconds.

The methods of the present invention further encompass providing a human cell culture enriched in neural cells, comprising the formation of an embryoid body that comprises a human pluripotent cell culture that expresses SSEA3, SSEA4, Oct4, Tra-1-60, Tra-1-80, and expresses nestin substantially uniformly. In one embodiment, the human pluripotent cell culture is provided by culturing the cells in an essentially single cell culture. In one embodiment, the human pluripotent cell culture is provided using a protease passaging treatment. In another embodiment, the human pluripotent cell culture is provided using antibody selection and protease passaging treatment. In another embodiment, the human pluripotent cell culture is provided using antibody selection. In certain embodiments of the invention, the antibody selection is performed using an anti-SSEA4 antibody. In one embodiment, the protease passaging treatment comprises the use of Collagenase at a concentration of approximately 1 mg/ml for approximately 5 minutes, and the subsequent use of trypsin at a concentration of approximately 0.05% for approximately 30 seconds.

In a further preferred embodiment, the method of providing a human cell culture enriched in neural cells comprises the formation of an embryoid body by culturing a human pluripotent cell culture of the invention with an essentially serum free medium. In one embodiment, the essentially serum free medium is a MEDII conditioned medium as defined herein. In another embodiment, the essentially serum free medium is a minimal medium that optionally comprises proline. In a further embodiment, the embryoid body is formed in MEDII/FGF2 medium supplemented with DMEM:F12 for approximately 2-5 days, and the embryoid body is then cultured with minimal medium. In other embodiments, the embryoid body is subsequently cultured with one or more cell differentiation environments to produce a human neural cell or human cell culture enriched in neural cells, wherein each environment is appropriate to the cell types as they appear from the preceding cell type. It is to be understood that the absence of the term “differentiation” when describing a MEDII conditioned medium does not indicate that the MEDII conditioned medium can not also be considered a “differentiation” environment. In certain embodiments, the essentially serum free medium preferably is also essentially LIF free.

As used herein, the term “MEDII conditioned medium” refers to a medium comprising one or more bioactive components as described herein. In a preferred embodiment, the bioactive component is derived from a hepatic or hepatoma cell or cell line culture supernatant. The hepatic or hepatoma cell or cell line can be from any species, however, preferred cell lines are mammalian or avian in origin. The hepatic or hepatoma cell line can be selected from, but is not limited to, the group consisting of: a human hepatocellular carcinoma cell line such as a Hep G2 cell line (ATCC HB-8065) or Hepa-1c1c-7 cells (ATCC CRL-2026); a primary embryonic mouse liver cell line; a primary adult mouse liver cell line; a primary chicken liver cell line; and an extraembryonic endodermal cell line such as END-2 and PYS-2. A particularly preferred cell line is the Hep G2 cell line (ATCC HB-8065). A description of the isolation of an essentially serum free MEDII conditioned medium from a Hep G2 cell line is provided in Example 2 below. In one embodiment of the present invention, the MEDII conditioned medium is derived from a Hep G2 cell line and contains supplements of FGF-2.

As used herein, the terms “bioactive component” and “bioactive factor” refer to any compound or molecule that induces a pluripotent cell to follow a differentiation pathway toward an EPL cell or a neural cell. Alternatively, the bioactive component may act as a mitogen or as a stabilizing or survival factor for a cell differentiating towards an EPL cell or neural cell. A bioactive component from the conditioned medium may be used in place of the MEDII conditioned medium in any embodiment described herein. The isolation of a bioactive component of MEDII is shown below in Example 2. While the bioactive component may be as described below, the term is not limited thereto. The term “bioactive component” as used herein includes within its scope a natural or synthetic molecule or molecules which exhibit(s) similar biological activity, e.g. a molecule or molecules which compete with molecules within the conditioned medium that bind to a receptor on ES or EPL cells or their differentiation products in adherent culture, in embryoid bodies, or in nonadherent cultures, responsible for EPL or neural induction, and/or EPL or neural proliferation, and/or EPL or neural survival.

The MEDII conditioned medium described herein can comprise one or more bioactive components selected from the group consisting of a low molecular weight component comprising proline or a proline containing peptide; a biologically active fragment of any of the aforementioned proteins or components; and an analog of any of the aforementioned proteins or components. In one preferred embodiment, the bioactive component of the MEDII conditioned medium can be replaced, at least in part, by proline. Preferably proline is present in the cell culture medium at a concentration of from approximately 1 μM to approximately 10 M, more preferably from a concentration of from approximately 5 μM to approximately 1 M, more preferably from approximately 10 μM to approximately 500 mM, more preferably from approximately 10 μM to approximately 100 mM, and more preferably from approximately 25 μM to approximately 10 mM. In one embodiment, proline is present in the cell culture medium at a concentration of approximately 50 μM. In addition, the MEDII conditioned medium may further comprise a neural inducing factor.

The low molecular weight component of the MEDII conditioned medium can comprise one or more proline residues or a polypeptide containing proline residues. As used herein, the term “polypeptide” refers to any of various amides that are derived from two or more amino acids by combination of the amino group of one acid with the carboxyl group of another and usually obtained by partial hydrolysis of proteins. In a preferred embodiment, the low molecular weight component is L-proline or a polypeptide including L-proline. The proline containing polypeptide preferably has a molecular weight of less than approximately 5 kD, more preferably less than approximately 3 kD. In a further preferred embodiment, the low molecular weight component is a polypeptide of between approximately 2-11 amino acids, more preferably of between approximately 2-7 amino acids and most preferably approximately 4 amino acids. The proline containing polypeptide can be selected from, but is not limited to, the following polypeptides: Pro-Ala, Ala-Pro, Ala-Pro-Gly, Pro-OH-Pro, Pro-Gly, Gly-Pro, Gly-Pro-Ala, Gly-Pro-Glu, Gly-Pro-OH-Pro, Gly-Pro-Arg-Pro (SEQ ID NO:1), Gly-Pro-Gly-Gly (SEQ ID NO:2), Val-Ala-Pro-Gly (SEQ ID NO:3), Arg-Pro-Lys-Pro (SEQ ID NO:4), and Arg-Pro-Lys-Pro-Gln-Gln-Phe-Phe-Gly-Leu-MetOH (SEQ ID NO:5).

As used herein, “essentially serum free” refers to a medium that does not contain serum or serum replacement, or that contains essentially no serum or serum replacement. As used herein, “essentially” means that a de minimus or reduced amount of a component, such as serum, may be present that does not eliminate the improved bioactive neural cell culturing capacity of the medium or environment For example, essentially serum free medium or environment can contain less than 10, 9, 8, 7, 6, 5, 4, 3, 2, or 1% serum wherein the presently improved bioactive neural cell culturing capacity of the medium or environment is still observed. In preferred embodiments of the present invention, the essentially serum free medium does not contain serum or serum replacement.

As used herein, “essentially LIF free” refers to a medium that does not contain leukemia inhibitory factor (LIF), or that contains essentially no LIF. As used herein, “essentially” means that a de minimus or reduced amount of a component, such as LIF, may be present that does not eliminate the improved bioactive neural cell culturing capacity of the medium or environment. For example, essentially LIF free medium or environment can contain less than 100, 75, 50, 40, 30, 10, 5, 4, 3, 2, or 1 ng/ml LIF, wherein the presently improved bioactive neural cell culturing capacity of the medium or environment is still observed.

The present invention further contemplates a method of culturing a human pluripotent cell comprising the steps of: a) providing a human pluripotent cell, b) passaging the cell culture using a protease treatment to thereby disperse the cell to an essentially single cell culture, and c) culturing the essentially single cell culture in the presence of a feeder cell, in the presence of a conditioned medium, or in the presence of a minimal medium. In a further embodiment, the invention encompasses a method of producing a human cell culture enriched in neural cells comprising the steps of: a) providing a human pluripotent cell, b) passaging the cell culture using a protease treatment to thereby disperse the cell culture to an essentially single cell culture, c) culturing the essentially single cell culture in the presence of a feeder cell, in the presence of a conditioned medium, or in the presence of a minimal medium and d) forming an embryoid body comprising the essentially single cell culture by culturing the cell culture with an optionally essentially serum free medium, to thereby produce the human neural cell. In certain embodiments, the protease treatment comprises the sequential use of Collagenase and trypsin. In preferred embodiments of the above methods, the protease treatment comprises treating the cell culture with Collagenase at a concentration of approximately 1 mg/ml for approximately 5 minutes, and treating the cell culture with trypsin at a concentration of approximately 0.05% for approximately 30 seconds. In other embodiments, the essentially serum free medium is a MEDII conditioned medium. It is further contemplated that the MEDII conditioned medium is a Hep G2 conditioned medium. In another embodiment, the MEDII conditioned medium comprises one or more proline residues or a polypeptide containing proline residues. In one embodiment, proline is present at a concentration of approximately 50 μM. In a further embodiment, the essentially serum free medium comprises proline and FGF2.

In one embodiment of the present invention described above, the pluripotent cell or cell culture is cultured with a minimal medium. As used herein, the term “minimal medium” refers to a tissue culture medium that is preferably essentially free from FGF, proline, and/or MEDII. As used herein, “essentially free from FGF” or “essentially FGF free” refers to a tissue culture medium that contains less than approximately 10, 9, 8, 7, 6, 5, 4, 3, 2, 1, 0.5, 0.1, or 0.01 ng/ml of an FGF. Preferably, the minimal medium comprises less than 1 ng/ml of an FGF. As used herein, “essentially free from proline” or “essentially proline free” refers to a tissue culture medium that contains less than approximately 500 μM, 400 μM, 300 μM, 200 μM, 100 μM, 50 μM, 10 μM, 5 μM, or 1 μM of proline. In one embodiment, the minimal medium comprises less than 10 μM proline. In another embodiment, the minimal medium is supplemented with proline. When the minimal medium is supplemented with proline, preferably the proline is present at a concentration of less than 500 μM, 400 μM, 300 μM, 200 μM, 100 μM, 50 μM, 10 μM, 5 μM, or 1 μM of proline. In one embodiment, the minimal medium comprises approximately 50 μM proline. As used herein, “essentially free from MEDII” or “essentially MEDII free” refers to a tissue culture medium that contains less than approximately 50%, 40%, 30%, 20%, 10%, 5%, 4%, 3%, 2%, or 1% of MEDII, as defined herein. Preferably the tissue culture medium comprises less than 5% MEDII.

As used herein, an “essentially single cell culture” is a cell culture wherein during passaging, the cells desired to be grown are dissociated from one another, such that the majority of the cells are single cells, or two cells that remain associated (doublets). Preferably, greater than 50%, 60%, 70%, 80%, 90%, 95%, 96%, 97%, 98%, 99% or more of the cells desired to be cultured are singlets or doublets. The term encompasses the use of any method known now or later developed that is capable of producing an essentially single cell culture.

In a preferred embodiment of the above methods, a “feeder cell” is a cell that is co-cultured with a human pluripotent cell and maintains the human pluripotent cell in an undifferentiated or partially differentiated state. In a preferred embodiment of the above method, the conditioned medium is obtained from a feeder cell that maintains the human pluripotent cell in an undifferentiated or partially differentiated state. In one embodiment, the feeder cell is a mouse cell, such as a mouse embryonic fibroblast. In a preferred embodiment, the mouse embryonic fibroblast is mitotically inactivated, using methods well known to those of skill in the art. In another embodiment, the feeder cell is a human feeder cell. In certain embodiments, the human feeder cell is selected from the group consisting of a human fibroblast cell, a MRC-5 cell, a human embryonic kidney cell, a mesenchymal cell, an osteosarcoma cell, a keratinocyte, a chondrocyte, a Fallopian ductal epithelial cell, a liver cell, a cardiac cell, a bone marrow stromal cell, a granulosa cell, a skeletal muscle cell, and an aortic endothelial cell. In a more preferred embodiment the human feeder cell is selected from the group consisting of a skin keloid fibroblast cell, a fetal skin fibroblast cell, a bone marrow stromal cell, or a skeletal muscle cell.

The present invention contemplates that the feeder cell can be a freshly plated feeder cell. As used herein, the term “freshly plated” means that the feeder cell has been allowed to attach to the tissue culture dish for less than 2 days. In certain embodiments, the feeder cell has been plated for less than 18 hours, in other embodiments the feeder cell has been plated for less than 10 hours, in other embodiments the feeder cell has been plated for less than 6 hours, and in further embodiments, the feeder cell has been plated for less than 2 hours. In another embodiment, preferably the feeder cell has been plated for approximately 6 to 18 hours. In one embodiment, HESC cultures that have been cultured in an essentially single cell culture, such as by protease passaging and/or antibody selection are prepared for differentiation by seeding the cells at a defined density on feeder layers that are between approximately 6 to 18 hours old. In another embodiment, manually passaged HESC cultures are prepared for differentiation by seeding the cells at a defined density on feeder layers that are freshly plated. Seeding manually passaged HESCs on fresh feeder layers appears to cause a differentiation event that enables uniform neural rosette differentiation in suspension, and although morphological changes are not apparent, this may also have a positive influence on the neural and DA differentiation of bulk passaged HESC.

In a preferred embodiment, the pluripotent cell is a human cell. As used herein, the term “pluripotent human cell” encompasses pluripotent cells obtained from human embryos, fetuses or adult tissues. In one embodiment, the pluripotent human cell is a differentiating cell. In one preferred embodiment, the pluripotent human cell is a human pluripotent embryonic stem cell. In certain preferred embodiments, the human pluripotent embryonic stem cell is obtained from a domed human embryonic stem cell colony, a crater human embryonic stem cell colony, and a protease passaged human embryonic stem cell colony. In another embodiment the pluripotent human cell is a human pluripotent fetal stem cell, such as a primordial germ cell. In another embodiment the pluripotent human cell is a human pluripotent adult stem cell. As used herein, the term “pluripotent” refers to a cell capable of at least developing into one of ectodermal, endodermal and mesodermal cells. In one preferred embodiment, the pluripotent human cell is a differentiating human cell. As used herein the term “pluripotent” refers to cells that are totipotent and multipotent. As used herein, the term “totipotent cell” refers to a cell capable of developing into all lineages of cells. As used herein, the term “multipotent” refers to a cell that is not terminally differentiated. In one preferred embodiment the multipotent cell is a neural precursor cell and the multipotent cell culture is a neural precursor cell culture. The pluripotent human cell can be selected from the group consisting of a human embryonic stem (ES) cell; a human inner cell mass (ICM)/epiblast cell; a human primitive ectoderm cell, such as an early primitive ectoderm cell (EPL); and a human primordial germ (EG) cell. The human pluripotent cells of the present invention can be derived using any method known to those of skill in the art at the present time or later discovered. For example, the human pluripotent cells can be produced using de-differentiation and nuclear transfer methods. Additionally, the human ICM/epiblast cell or the primitive ectoderm cell used in the present invention can be derived in vivo or in vitro. EPL cells may be generated in adherent culture or as cell aggregates in suspension culture, as described in WO 99/53021, herein incorporated by reference.

As used herein, the term “protease passaged” cell refers to a cell that has been passaged using a protease treatment such that the cells were cultured as an essentially single cell culture. In one embodiment, the protease treatment comprises the sequential use of Collagenase and trypsin, however, other protease treatments known now or later developed are encompassed within the term.

The present invention further contemplates the human neural cell or human cell culture enriched in neural cells produced by any of the above-described methods. As used herein, the term “neural cell” includes, but is not limited to, a neurectoderm cell; an EPL derived cell; a glial cell; a neural cell of the central nervous system such as a dopaminergic cell, a differentiated or undifferentiated astrocyte or oligodendrocyte; a neural stem cell, a neural progenitor, a glial progenitor, an oligodendrocyte progenitor, and a neural cell of the peripheral nervous system. As used herein, the term “neurectoderm” refers to undifferentiated neural progenitor cells substantially equivalent to cell populations comprising the neural plate and/or neural tube; or a partially differentiated neural progenitor cell. Neurectoderm cells are multipotential. Therefore, “neural cell” as used in the context of the present invention, is meant that the cell is at least more differentiated towards a neural cell type than the pluripotent cell from which it is derived.

A central characteristic of the neural differentiation method described herein is that the medium in which embryoid bodies are formed is preferably essentially serum free, and that cell-cell interactions are not chemically disrupted after the formation of an embryoid body. It is likely that serum induces the formation of primitive endoderm in embryoid body differentiation, which would direct primitive ectoderm equivalents to non-neural fates. Therefore, in essentially serum free conditions, HESCs are not co-opted from their proposed default pathway of neural differentiation. In the system of the present invention, intact HESC colonies are harvested and placed in suspension, and sfEBs, sfEBMs, and sfEBPs are passaged by being cut into ˜200 μm pieces rather than by disaggregation to single cells. Without being limited to a theory, it is possible that factors known to be critical in the induction of dopaminergic differentiation, such as En1, Nurr1, Pitx3, and Lmx1b are expressed in HESCs or during intrinsic neural differentiation, and are not disrupted by breaking cell-cell communication, which influences the majority of neurons to a dopaminergic fate. Expression of these molecules has been shown to be upregulated in essentially serum free conditions in comparison to serum containing conditions.

One distinguishing feature of the approach of the present invention is that the efficient generation of TH+ neurons appears to be an intrinsic component of differentiation, as opposed to other documented approaches that rely on the induction of DA differentiation via co-culture with a stromal cell layer, addition of FGF8 and shh, or overexpression of the Nurr1 transgene. Given the intrinsic DA differentiation capacity in this system, co-culture of ES cells on a stromal cell layer may not necessarily provide inductive signals, but rather an appropriate matrix that enables ES cell survival, ES cell-ES cell interaction and development along an intrinsic DA differentiation pathway. Zhang et al. (Nat Biotech 2001, 19: 1129-1133) used a suspension culture system for the neural differentiation of HESC, but only detected “a small number of neurons” that expressed TH. There are numerous differences in the culture methodologies that were developed through empirical observations that could have contributed to the significantly superior DA differentiation approach of the present invention. As used herein, depending on the context, the term “DA” refers to either dopaminergic, or dopamine.

Briefly, one present method of passaging HESCs involves routine disruption of HESC colonies to essentially single cells, and optionally, periodic SSEA4 selection to remove differentiated cells. Zhang et al. used a technique where enzymatic digestion with collagenase and dispase was used to break HESC colonies into pieces for passaging, rather than disaggregation to essentially single cells (Nat Biotech 2001, 19: 1129-1133). This approach could lead to the gradual accumulation of differentiated cells within the culture, as there is no selection against differentiation, unlike morphological criteria with manual passaging and the bulk passaging and magnetic sorting methods described herein. Heterogeneous HESC populations with stochastic levels of differentiated cells could lead to the generation of cell types that generate inhibitory signals for DA differentiation within an embryoid body, such that the neural rosettes generated are fated to alternate neuronal differentiation pathways. The selection and passaging procedures described herein leads to a far more homogeneous pluripotent cell population than observed in manually passaged HESCs and is also more likely to be homogeneous than the culture of Zhang et al. (Nat Biotech 2001, 19: 1129-1133). Therefore, the methods described herein would cause minimal disruption of an intrinsic neural and DA differentiation pathway, which would lead to the efficient and robust DA differentiation observed herein.

In the same manner, the differentiation protocol reported by Ron McKay's laboratory (Kim et al., Nature 2002 418(6893):50-6) includes serum in the initial embryoid body suspension culture, and does not produce neural progenitors with a high intrinsic DA differentiation potential. That these cells respond to FGF8/shh demonstrates that the DA differentiation capacity of these neural precursors is not lost, but that a possible default specification to DA differentiation may have been altered by the presence of other cell types within these embryoid bodies. This approach also requires the high level expression of a Nurr1 transgene to achieve reliable and significant neural differentiation down a dopaminergic pathway.

Furthermore, Zhang et al. cultured embryoid bodies in 20% knockout serum replacement (KSR) medium for four days after derivation (Nat. Biotech 2001, 19:1129-1133). KSR is a media supplement containing amino acids, ascorbic acid, transferrin, insulin, albumin, trace elements and trace element moiety-containing compounds. At a 1× formulation, added at 15% to base medium, KSR contributes 5.21 mM L-proline to the medium. It is likely that these various components would contribute to the differentiation and survival of numerous non-neural cell types, the signaling from which could also inhibit the generation of neural rosettes capable of intrinsic DA differentiation. Zhang et al. seeded embryoid bodies and subsequently purified rosettes from a background of undefined cell types by differential response to dispase (Nat. Biotech 2001, 19: 1129-1133). The approach described herein is serum and KSR free from the point of embryoid body derivation and onwards. In the conditions described herein, serum free embryoid bodies cultured in minimal and proline conditions exhibited a high degree of cell death over the first few weeks. The interpretation is that cells were being continuously generated that could not survive in the minimal conditions, and neural rosette cells were presumably generated in the absence of other cell types that would survive in more complete media. In L-proline conditions at least, the large majority of neurons generated were TH+, greater than 50% of the cells in an embryoid body in suspension. In serum free embryoid bodies cultured in DMEM/F12 with FGF2 or FGF2/MEDII, low cell death was exhibited, but the proportion of neurons that were TH+ remained high. Therefore cell types capable of inhibiting the intrinsic DA differentiation capacity of rosette cells were not generated in significant numbers in these conditions. One explanation of these results is HESCs have uniform converted to rosette cells and their presumed daughter cells, differentiating neurons and glial like cells, as suggested by sectioning of sfEBMs. However, the rosettes generated by Zhang et al. did not readily differentiate to DA neurons (Nat. Biotech 2001, 19: 1129-1133). This may suggest that cells of non-neural lineages persist from their BESC cultures, or are generated in the first 4 days of their suspension culture system, survive due to the presence of the KSR supplement and have a negative influence on the DA differentiation of early rosette cells.

It also appears that L-proline plays a role in neural differentiation, however, the precise role that it plays to enhance the survival or proliferation or neural cells within this differentiation system is unclear. L-proline is not an essential amino acid and can be generated biosynthetically within cells, for example from ornithine, a component of the Krebs cycle, by ornithine cyclodeaminase. Besides incorporation into peptides, functional roles for L-proline in lipogenesis, glycogen synthesis, cell growth and as a neuromodulator in the CNS have been reported (Baqet et al., 1991, Biochem. J, 273; 57-62; Sugden et al., 1984, Biochim, Biophys. Acta 798; 368-373; Houck and Michalopoulos, 1985, In Vitro Cell Dev. Biol., 21; 121-124; Fremeau et al., 1992, Neuron, 8; 915-925). Cells can also import a large proportion of the L-proline they require, which is mediated via a transport system for short-chain amino acids (Collinari and Oxender 1987, Ann Rev. Nutr., 7; 75-90; McGivan and Pastor-Anglada 1994, Biochem. J., 299; 321-334).

L-proline is a common component of many cell culture formulations, for example, it is present at 150 μM in DMEM/F12 and at 5.21 mM in 1×KSR (15%). However, no other reports examining neural differentiation of ES cells have highlighted the role that L-proline may play, and other ES research groups are clearly oblivious to this effect. Carpenter et al. (Expt. Neurol. 2001, 172;383-397) cultured HESCs in 20% KSR with approximately 6.94 mM L-Proline, and differentiated embryoid bodies in 20% serum and 1% non essential amino acids (100 μM L-Pro). Zhang et al. (Nat Biotech 2001, 19; 1129-1133) cultured HESC in 20% KSR and differentiated embryoid bodies for 4 days in 20% KSR, before culturing neural progenitors in DMEM/F12. Ying et al. (2003 Nat. Biotech. 21:183-186) generated neural progenitors from mouse ES cells by culturing in an apparently minimal medium, but this medium contained 150 μM L-proline. Reubinoff et al. (Nat Biotech 2001, 19; 1134-1140) overgrew HESCs in 20% FCS and non-essential amino acids, and grew neural progenitors in DMEM/F12. Kawasaki et al. (2000, Neuron 28; 31-40) and Kawasaki et al. (2002, PNAS 99; 1580-1585) differentiated mouse and primate ES cells, respectively, in contact with PA6 cells in the presence of 10% KSR. Rathjen et al. (2002, Development 129; 2649-2661) differentiated mouse ES cells in the presence of 10% FCS for 5 days, followed by further culture in DMEM/F12. Therefore, in all these examples, ES cell differentiation was carried out in the presence of a minimum of 100 μM and a maximum of 7 mM L-proline. The minimal medium differentiations documented herein represent the first differentiation of ES cells, and particularly of human ES cells, to neurons in a L-proline free environment, and clearly demonstrate a survival/proliferation effect upon addition of this amino acid.

Functional roles for L-proline in neural differentiations could include extracellular or intracellular effects. Extracellular activities could include, for example, interaction with specific signaling receptors and subsequent signal transduction, or modulation of the extracellular matrix or the cell membrane. Given that the effects observed in this system are in the range of 50 to 500 μM L-proline, or possibly 7 mM L-proline in KSR conditions in the differentiation systems reported elsewhere, it may be more likely that the role that L-proline is functioning in an intracellular metabolic role.

Three components of the proline and neutral amino acid transport system have been described, and they share ˜50% peptide sequence similarity (System A amino acid transporters: SAT1, SAT2 and ATA3). SAT1 and SAT2 are sodium-coupled, pH sensitive high affinity transporters and are expressed in the brain, or ubiquitously, respectively. ATA3 is a low affinity transporter that is sodium independent and expressed primarily in the liver. Ensena et al. (2001 Biochem J. 360; 507-512) examined L-proline transport in vascular smooth muscle cells that predominantly express the SAT1 transporter in response to TGF-β1. They showed that the importation of L-proline by SAT1 could be inhibited or competed by the neutral amino acids P, F, S, A, C, T, M, V, Q, G, I, Y, L, and the basic amino acid histidine, but not by the anionic or cationic amino acids K, R, E, D. This indicated that the net uptake of L-proline within a cell culture system will be affected by the ratio of L-proline to neutral amino acids in the media formulation, that can be imported by the same transporters. The expression of SAT1 in the brain may be relevant to the effect of L-proline observed here on neural differentiation from HESC in vitro.

There are several roles that L-proline could be playing in neural progenitor cells and neurons in our system. Effects on cell survival could be based on an anti-apoptotic or anti-oxidant activity. Another possibility is that L-proline is a significant alternate energy source in neural cultures. L-glutamine is an essential amino acid and is commonly thought to be a component of most cell media formulations because it cannot be produced by mammalian cells for the incorporation into peptides. However L-glutamine is also an important energy source, supplying a significant proportion of the available energy in media formulations. L-glutamine can be converted to glutamate by Glutaminase, which can then be converted to α-ketogluterate, a component of the Krebs cycle, by glutamate dehydrogenase. This reaction generates one NADPH (yield 3 ATP from oxidative phosphorylation) and generates a NH⁺₄. L-proline can be converted to glutamate y-semialdehyde by Proline Oxidase and an uncatalyzed reaction, and then to glutamate by Glutamate Semialdehyde Dehydrogenase, a reaction that yields one NADPH. Inside the mitochondria, glutamate and oxaloacetate can be reacted by Aspartate Aminotransferase to generate α-ketogluterate and aspartate. Conversely, Glutamate Dehydrogenase can convert glutamate to α-ketogluterate, generating a NADPH and a NH⁺₄. For each α-ketogluterate generated, 2 NADPH, one FADH₂ and one GTP can be generated in the Krebs cycle (equivalent to a final 9 ATP). Aspartate can be processed through the urea cycle for the yield of one fumarate, which can yield one NADPH in the Krebs cycle (yield 3 ATP). Each NH⁺₄ generated can be processed through the urea cycle for the cost of 4 phosphate bonds, and the yield of 1 fumarate (net loss of 1 ATP).

Therefore, L-proline could provide an alternate and important energy source for neural cultures, as a single proline molecule can yield 14 or 15 ATP compared to 10 or 11 for glutamine, depending on the processing pathway of the glutamate intermediate. It is possible that neural cultures could be specialized to utilize this pathway compared to other cell types. Given the exposure of manually and bulk passaged HESCs to high levels of L-proline from the KSR, there may be some preconditioning for the utilization of L-proline as an energy source in this differentiation system. A precedence for the use of L-proline in this manner has been demonstrated in Trypanosoma cruzi, where L-proline may be used as the main energy and carbon source, in particular in the insect vector stage (Evans and Brown, 1972, J. Protozool. 19; 686-690; Silber et al., 2002, J. Eukaryot. Microbiol. 49(6); 441-446). However, in this example only the α-ketogluterate to succinate segment of the Krebs cycle appears to be utilized (van Weelden et al., 2003, JBC in press, Manuscript M213190200). This can potentially generate a net yield of 10 ATP per input L-proline molecule (2 NADPH, 1 ATP, and net 3 ATP from the processing of aspartate to fumarate through the urea cycle). A potential role for L-proline as a neural inducer is not clear, as demonstrated by the differentiation to neurons in the absence of proline. However, the trypsin passaged HESCs were cultured in 20% KSR, which contains 6.9 mM L-proline. Immunostaining of manually passaged HESCs cultured in 5% KSR (1.7 mM Proline) demonstrated expression of the SAT1 proline transporter, which indicated these cells could already be responding to the high proline concentration in the medium. It is possible that the uniform nestin expression observed in bulk passaged HESCs is indicative of a pre-neural character of these cells which otherwise express the expected pluripotent cell markers. Regardless, the uniformity of this HESC starting population is likely to be a key factor in the efficiency of DA differentiation observed herein.

The present invention further contemplates the use of a composition comprising an amphiphilic lipid compound. In a preferred embodiment, the amphiphilic lipid compound is selected from the group consisting of a ceramide compound, a sphingosine compound, and a hydroxyalkyl ester compound. In one embodiment, the embryoid body comprising the pluripotent human cell is cultured with a composition comprising the amphiphilic lipid compound.

In a preferred embodiment, the amphiphilic lipid compound is a ceramide compound, wherein the ceramide compound is a N-acyl derivative of β-hydroxyalkylamine. In a preferred embodiment, the ceramide compound has the general formula 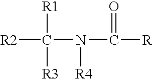
and, wherein R is a saturated or mono- or polyunsaturated (cis or trans) alkyl group having greater than 2 carbon atoms; R1, R2, R3, and R4 may be the same or different and are saturated or mono- or polyunsaturated hydroxylated alkyl groups, aryl groups, or hydrogen. In one embodiment, R4 is an alkyl chain having from 1 to 12 carbon atoms. In a preferred embodiment, R is a saturated or mono- or polyunsaturated (cis or trans) alkyl group having from 12-20 carbon atoms, the hydroxylated alkyl groups have from 1-6 carbon atoms, R1 and R2 are hydroxylated alkyl groups, and R3 is hydrogen. In one embodiment, the composition comprises a ceramide compound of the structure 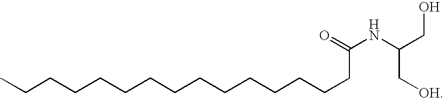
In another preferred embodiment, the composition comprises a ceramide compound of the structure

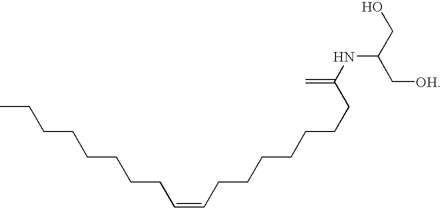

In another embodiment, the present invention contemplates the use of a composition comprising a sphingosine compound, wherein the sphingosine compound has the general formula 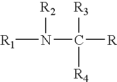
and, R is a saturated or mono- or polyunsaturated (cis or trans) alkyl group having greater than 2 carbon atoms; R1, R2, R3, and R4 may be the same or different and are saturated or mono- or polyunsaturated hydroxylated alkyl groups, aryl groups, or hydrogen. In preferred embodiments, the sphingosine compound is selected from the group comprising D-erythro-sphingosine, L-threo-sphingosine, dimethylsphingosine, and N-oleoyl ethanolamine.

In another embodiment, the present invention contemplates the use of a composition comprising a hydroxyalkyl ester compound, wherein the hydroxyalkyl ester compound has the general formula 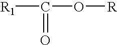
and, wherein R is a saturated or mono- or polyunsaturated (cis or trans) alkyl group having greater than 2 carbon atoms; and R1 is a saturated or mono- or polyunsaturated hydroxylated alkyl group, aryl group, or hydrogen. In a preferred embodiment, the hydroxyalkyl ester compound is an O-acyl derivative of gallic acid. In another preferred embodiment, the hydroxyalkyl ester compound is the n-dodecyl ester of 3,4,5-trihydroxybenzoic acid (“laurylgallate”), which has the formula

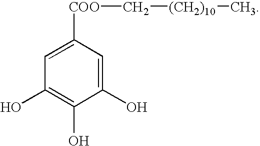

In preferred embodiments of the present invention, the composition comprises a ceramide compound selected from the group consisting of N-(2-hydroxy-1-(hydroxymethyl)ethyl)-palmitoylamide (“S16”); N-(2-hydroxy-1-(hydroxymethyl)ethyl)-oleoylamide (“S18”); N,N-bis(2-hydroxyethyl)palmitoylamide (“B16”); N,N-bis(2-hydroxyethyl)oleoylamide (“B18”); N-tris(hydroxymethyl)methyl-palmitoylamide (“T16”); N-tris(hydroxymethyl)methyl-oleoylamide (“T18”); N-acetyl sphingosine (“C2-ceramide”); D-threo-1-phenyl-2-decanoylamino-3-morpholino-1-propanol (“D-threo-PDMP”); D-threo-1-phenyl-2-hexadecanoylamino-3-morpholino-1-propanol (“D-Threo-PPMP”); D-erythro-2-tetradecanoyl-1-phenyl-1-propanol (“D-MAPP”); D-erythro-2-(N-myristoylamino)-1-phenyl-1-propanol (“MAPP”), and N-hexanoylsphingosine (C6-ceramide).

Those of skill in the art will recognize that many other variations of the general formulas above exist, and that the use of all such variations is encompassed by the methods of the present invention. In more preferred embodiments, the ceramide compound is selected from the group comprising S16, S18 and functional homologues, isomers, and pharmaceutically acceptable salts thereof. In a preferred embodiment the ceramide compound is S18. In another preferred embodiment the ceramide compound is S16. In another preferred embodiment, the amphiphilic lipid compound can include the metabolites and catabolites of the ceramide compound, the sphingosine compound, and the hydroxyalkyl ester compound. The composition comprising the amphiphilic lipid compound may further comprise pharmaceutically acceptable carriers, excipients, additives, preservatives, and buffers.

In the methods of the present invention, it is preferred that the concentration of the amphiphilic lipid compound is from approximately 0.1 μM to 1000 μM, more preferred that the concentration of the amphiphilic lipid compound is from approximately 1 μM to 200 μM, and more preferred that the concentration of the amphiphilic lipid compound is from approximately 8 μM to 100 μM, and most preferred that the concentration of the amphiphilic lipid compound is approximately 100 μM.

In the methods of the present invention, it is preferred that the duration of culturing the differentiating human pluripotent cell with the amphiphilic lipid compound is from approximately 1 hour to 20 days, more preferably from approximately 6 hours to 10 days, and most preferably from approximately 12 hours to 6 days.

In a further embodiment, a subsequent cell differentiation environment comprises an amphiphilic lipid compound. In a preferred embodiment, the amphiphilic compound is selected from the group comprising a ceramide compound, a sphingosine compound, and an hydroxyalkyl ester. In more preferred embodiments, the ceramide compound is a ceramide analog of the serinol type selected from the group comprising S16, S18 and functional homologues, isomers, and pharmaceutically acceptable salts thereof. In a preferred embodiment the ceramide compound is S18. In another preferred embodiment the ceramide compound is S16. In a preferred embodiment, the composition comprising the amphiphilic lipid compound is essentially serum free.

In the methods of the present invention, the composition comprising the ceramide compound further comprises a MEDII conditioned medium. In a further embodiment, the composition comprising the ceramide compound is essentially serum free. In another embodiment, the composition comprising the ceramide compound further comprises serum, or a serum replacement.

In another preferred embodiment of the above methods, an embryoid body is formed upon culturing the pluripotent human cell or cell culture with an essentially serum free medium, wherein the serum free medium is optionally a MEDII conditioned medium, the embryoid body is seeded, and cultured with a composition comprising the amphiphilic lipid compound until the human neural cell is produced or the human cell culture enriched in neural cells is produced. In another embodiment, the embryoid body is formed upon culturing the pluripotent human cell or cell culture with a medium, the embryoid body is seeded, and cultured with a composition comprising the amphiphilic lipid compound until the human neural cell is produced or the human cell culture enriched in neural cells is produced. In one embodiment, the amphiphilic lipid compound is in an essentially serum free medium. In a further embodiment, the essentially serum free medium comprises a MEDII conditioned medium, proline, or a proline containing polypeptide. In other embodiments, the amphiphilic lipid compound is in a serum containing medium. In other preferred embodiments, the seeded embryoid body is subsequently cultured with one or more cell differentiation environments to produce a human neural cell or human cell culture enriched in neural cells, wherein each environment is appropriate to the cell types as they appear from the preceding cell type. The amphiphilic lipid compound is selected from the group consisting of a ceramide compound, a sphingosine compound, and a hydroxyalkyl ester compound. In a preferred embodiment, the amphiphilic lipid compound is a ceramide compound of the serinol type.

As used herein, the term “cell differentiation environment” refers to a cell culture condition wherein the pluripotent cells or embryoid bodies derived therefrom are induced to differentiate into neural cells, or are induced to become a human cell culture enriched in neural cells. Preferably the neural cell lineage induced by the growth factor will be homogeneous in nature. The term “homogeneous,” refers to a population that contains more than 80%, 85%, 86%, 87%, 88%, 89%, 90%, 91%, 92%, 93%, 94%, 95%, 96%, 97%, 98%, or 99% of the desired neural cell lineage.

In one embodiment, the cell differentiation environment comprises an amphiphilic lipid compound. In a further embodiment, the amphiphilic lipid compound is a ceramide compound. In another embodiment, the cell differentiation environment is a suspension culture. As used herein, the term “suspension culture” refers to a cell culture system whereby cells are not tightly attached to a solid surface when they are cultured. Non-limiting examples of suspension cultures include agarose suspension cultures, and hanging drop suspension cultures. In one embodiment, the cell differentiation environment comprises a suspension culture where the tissue culture medium is Dulbecco's Modified Eagle's Medium and Ham's F12 media (DMEM/F12), and it is supplemented with a fibroblast growth factor (FGF) such as FGF-2. In a preferred embodiment, the cell differentiation environment comprises an FGF. In a preferred embodiment, the cell differentiation environment comprises a suspension culture where the tissue culture medium is DMEM/F12, FGF-2, and MEDII conditioned medium. In a preferred embodiment, the suspension culture is an agarose suspension culture. In certain other embodiments, the cell differentiation environment is essentially free of human leukemia inhibitory factor (hLIF). In certain other embodiments the cell differentiation environment is a minimal medium as defined herein.

In other embodiments, the cell differentiation environment can also contain supplements such as L-Glutamine, NEAA (non-essential amino acids), P/S (penicillin/streptomycin), N2 supplement (5 μg/ml insulin, 100 μg/ml transferrin, 20 nM progesterone, 30 nM selenium, 100 μM putrescine (Bottenstein, and Sato, 1979 PNAS USA 76, 514-517) and β-mercaptoethanol (β-ME). It is contemplated that additional factors may be added to the cell differentiation environment, including, but not limited to fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, and amnionless. TGF, BMP, and GDF antagonists could also be added in the form of TGF, BMP, and GDF receptor-Fc chimeras. Other factors that may be added include molecules that can activate or inactivate signaling through Notch receptor family, including but not limited to proteins of the Delta-like and Jagged families as well as gamma secretase inhibitors and other inhibitors of Notch processing or cleavage. Other growth factors may include members of the insulin like growth factor family (IGF), the wingless related (WNT) factor family, and the hedgehog factor family. Additional factors may be added to promote neural stern/progenitor proliferation and survival as well as neuron survival and differentiation. These neurotrophic factors include but are not limited to nerve growth factor (NGF), brain derived neurotrophic factor (BDNF), neurotrophin-3 (NT-3), neurotrophin-4/5 (NT-4/5), interleukin-6 (IL-6), ciliary neurotrophic factor (CNTF), leukemia inhibitory factor (LIF), cardiotrophin, members of the transforming growth factor (TGF)/bone morphogenetic protein (BMP)/growth and differentiation factor (GDF) family, the glial derived neurotrophic factor (GDNF) family including but not limited to neurturin, neublastin/artemin, and persephin and factors related to and including hepatocyte growth factor. Neural cultures that are terminally differentiated to form post-mitotic neurons may also contain a mitotic inhibitor or mixture of mitotic inhibitors including but not limited to 5-fluoro 2′-deoxyuridine and cytosine β-D-arabino-furanoside (Ara-C). The cell differentiation environment can further comprise conditions that are known to lead to an increase in endogenous ceramide levels, including but not limited to ionizing radiation, UV light radiation, application of retinoic acid, heat shock, chemotherapeutic agents such as but not limited to daunorubicin, and oxidative stress. Endogenous ceramide levels can also be elevated by incubating the cells in medium containing a sphingomyelinase or a compound with similar activity, or by treating the cells with an inhibitor of ceramidase such as N-oleoylethanolamine.

In another embodiment, the cell differentiation environment can contain compounds that enhance the activity of the amphiphilic lipid compound. In an alternative embodiment, the cell differentiation environment can contain other inducers or enhancers of apoptosis that synergize with the activity of the amphiphilic lipid compounds. In a further embodiment, the cell differentiation environment can comprise compounds that make the neural cells more resistant to apoptosis. In this embodiment, the addition of compounds that increase the resistance of neural cells to amphiphilic lipid compound enhanced apoptosis allows for the use of higher levels of the amphiphilic lipid compounds. As used herein, the term “higher levels” refers to concentrations of the amphiphilic lipid compound that would inhibit the growth or differentiation of neural cells in the absence of the additional compound, but that do not inhibit the growth or differentiation in the presence of the additional compound.

In other embodiments, the cell differentiation environment comprises seeding the embryoid body to an adherent culture. As used herein, the terms “seeded” and “seeding” refer to any process that allows an embryoid body or a portion of an embryoid body to be grown in adherent culture. An used herein, the term “a portion” refers to at least one cell from an embryoid body, preferably between approximately 1-10 cells, more preferably between approximately 10-100 cells from an embryoid body, and more preferably still between approximately 50-1000 cells from an embryoid body. As used herein, the term “adherent culture” refers to a cell culture system whereby cells are cultured on a solid surface, which may in turn be coated with a substrate. The cells may or may not tightly adhere to the solid surface or to the substrate. The substrate for the adherent culture may further comprise any one or combination of polyornithine, laminin, poly-lysine, purified collagen, gelatin, extracellular matrix, fibronectin, tenascin, vitronectin, poly glycolytic acid (PGA), poly lactic acid (PLA), poly lactic-glycolic acid (PLGA) and feeder cell layers such as, but not limited to, primary astrocytes, astrocyte cell lines, glial cell lines, bone marrow stromal cells, primary fibroblasts or fibroblast cells lines. In addition, primary astrocyte/glial cells or cell lines derived from particular regions of the developing or adult brain or spinal cord including but not limited to olfactory bulb, neocortex, hippocampus, basal telencephalon/striatum, midbrain/mesencephalon, substantia nigra, cerebellum or hindbrain may be used to enhance the development of specific neural cell sub-lineages and neural phenotypes. Furthermore, the substrate for the adherent culture may comprise the extracellular matrix laid down by a feeder cell layer, or laid down by the pluripotent human cell or cell culture.

In other embodiments of the present invention, it is not required that an embryoid body is formed upon culturing the pluripotent human cell or cell culture. In these embodiments, a pluripotent human cell or cell culture is optionally selected with an anti-SSEA4 antibody, passaged using a protease treatment, cultured with a medium, and as an optional additional step, the resultant cells are cultured with a composition comprising an amphiphilic lipid compound to produce a human neural cell or human cell culture enriched in neural cells. Alternatively, the pluripotent human cell or cell culture is optionally selected with an anti-SSEA4 antibody, passaged such that the cell culture is in an essentially single cell culture, cultured with a medium, and as an additional optional step, the resultant cells are cultured with a composition comprising an amphiphilic lipid compound to produce a human neural cell or human cell culture enriched in neural cells. In some embodiments, prior to culturing the cell with the composition comprising the amphiphilic lipid compound, the pluripotent human cell is first cultured with an essentially serum free medium. In other embodiments, the essentially serum free medium comprises MEDII conditioned medium or the bioactive component of a MEDII conditioned medium. In still other embodiments, the cells cultured with the amphiphilic lipid compound are subsequently cultured with one or more cell differentiation environments to produce a human neural cell or human cell culture enriched in neural cells, wherein each medium is appropriate to the cell types as they appear from the preceding cell type. In a preferred embodiment, the amphiphilic lipid compound is selected from the group consisting of a ceramide compound, a sphingosine compound, and a hydroxyalkyl ester compound. In a preferred embodiment, the amphiphilic lipid compound is a ceramide compound of the β-hydroxyalkylamine type.

The present invention further contemplates methods of enhancing the efficiency of the transplantation of a cultured human pluripotent cell or cell culture, comprising the steps of (a) culturing a human pluripotent cell with a growth medium comprising a ceramide compound of the general formula described above, wherein R is a saturated or mono- or polyunsaturated (cis or trans) alkyl group having greater than 2 carbon atoms, and R1, R2, R3, and R4 may be the same or different and are saturated or mono- or polyunsaturated hydroxylated alkyl groups, aryl groups, or hydrogen; and (b) transplanting the cultured human pluripotent cell or cell culture into the patient. In one embodiment, R4 is an alkyl chain having from 1 to 12 carbon atoms. In a preferred embodiment, R is a saturated or mono- or polyunsaturated (cis or trans) alkyl group having from 12-20 carbon atoms, the hydroxylated alkyl groups have from 1-6 carbon atoms, and R1 and R2 are hydroxylated alkyl groups. In other preferred embodiments, the ceramide compound is selected from the group comprising S16, S18 and functional homologues, isomers, and pharmaceutically acceptable salts thereof. In a preferred embodiment the ceramide compound is S18. In another preferred embodiment the ceramide compound is S16. In a preferred embodiment of the above method, the cell population comprising the cultured human pluripotent cell contains at least 80% of a neural cell.

The present invention further contemplates a composition for promoting maintenance, proliferation, or differentiation of a human neural cell, the composition comprising a cell culture medium comprising MEDII conditioned medium or the bioactive component of a MEDII conditioned medium and an amphiphilic lipid compound of the general formulas described above. Preferably the amphiphilic lipid compound is selected from the group consisting of the ceramide compound, the sphingosine compound, and the hydroxyalkyl ester compound of the formulas described above. In a preferred embodiment, the amphiphilic lipid compound is a ceramide compound of the β-hydroxyalkylamine type, wherein R is a saturated or mono- or polyunsaturated (cis or trans) alkyl group having from 12-20 carbon atoms, the hydroxylated alkyl groups have from 1-6 carbon atoms, and R1 and R2 are hydroxylated alkyl groups. In one embodiment, the ceramide compound is selected from the group consisting of N-(2-hydroxy-1-(hydroxymethyl)ethyl)-palmitoylamide (“S16”); N-(2-hydroxy-1-(hydroxymethyl)ethyl)-oleoylamide (“S18”); N,N-bis(2-hydroxyethyl)palmitoylamide (“B16”); N,N-bis(2-hydroxyethyl)oleoylamide (“B18”); N-tris(hydroxymethyl)methyl-palmitoylamide (“T16”); N-tris(hydroxymethyl)methyl-oleoylamide (“T18”); N-acetyl sphingosine (“C2”); D-threo-1-phenyl-2-decanoylamino-3-morpholino-1-propanol (“D-threo-PDMP”); D-threo-1-phenyl-2-hexadecanoylamino-3-morpholino-1-propanol (“D-Threo-PPMP”); D-erythro-2-tetradecanoyl-1-phenyl-1-propanol (“D-MAPP”); D-erythro-2-(N-myristoylamino)-1-phenyl-1-propanol (“MAPP”); and N-hexanoylsphingosine (C6-ceramide). In more preferred embodiments, the ceramide compound is selected from the group comprising S16, S18 and functional homologues, isomers, and pharmaceutically acceptable salts thereof. In a preferred embodiment the ceramide compound is S18. In another preferred embodiment the ceramide compound is S16. In other embodiments, the amphiphilic lipid compound is a sphingosine compound, wherein the sphingosine compound is selected from the group consisting of D-erythro-sphingosine, L-threo-sphingosine, dimethylsphingosine, and N-oleoyl ethanolamine. In other embodiments, the amphiphilic lipid compound is a hydroxyalkyl ester compound, wherein the hydroxyalkyl ester is laurylgallate. The composition comprising the amphiphilic lipid compound may further comprise pharmaceutically acceptable carriers, excipients, additives, preservatives, and buffers. The invention also contemplates the neural cell or human cell culture enriched in neural cells that is cultured in the composition.

The MEDII conditioned medium described herein can comprise one or more bioactive components selected from the group consisting of a low molecular weight component; a biologically active fragment of any of the aforementioned proteins or components; and an analog of any of the aforementioned proteins or components. The bioactive component can be a neural inducing factor, and in a preferred embodiment, is isolated from MEDII conditioned medium using purification techniques well known in the art. At each step of the purification procedure the samples or fractions are applied the pluripotent cell to test for the presence of the neural inducing factor. The bioactive component can be proline or a proline containing peptide

The step of culturing the human pluripotent cells with the MEDII conditioned medium to produce embryoid bodies (EBs) or EPL cells can be conducted in any suitable manner. For example, EPL cells may be generated in adherent culture or as cell aggregates in suspension culture. EBs may be generated in suspension culture using the hanging drop technique or by culturing the cells on agarose coated plates. EBs can be generated in serum containing medium, or in essentially serum free medium. It is also to be understood that the step of culturing the embryoid body with an essentially serum free medium and/or an essentially serum free cell differentiation environment can also be conducted in any manner known to those of skill in the art. In one embodiment, the embryoid body is initially generated in serum containing medium and then transferred to an essentially serum free medium for further neural differentiation and ceramide treatment.

As stated above, the present invention provides a method of producing a neural cell or producing a human cell culture enriched in neural cells comprising the steps of: a) providing a pluripotent human cell; b) culturing the pluripotent human cell with an essentially serum free medium to form an embryoid body; and c) culturing cells from the embryoid body with a composition comprising a ceramide compound to produce the neural cell or the human cell culture enriched in neural cells. In one embodiment, the essentially serum free medium is a MEDII conditioned medium. It is to be understood that the step of culturing the pluripotent cell with the essentially serum free MEDII conditioned medium can include the use of a “normal” or “other” essentially serum free medium supplemented with a MEDII conditioned medium. The “normal” or “other” medium, such as a normal human ES medium, can be supplemented with an essentially serum free MEDII conditioned medium at any concentration, but it is preferred that the “normal” or “other” medium can be supplemented at between approximately 10-75%, more preferably between approximately 40-60% and most preferably approximately 50% essentially serum free MEDII conditioned medium. The “normal” or “other” medium that is supplemented with essentially serum free MEDII conditioned medium is also preferably essentially serum free, containing no or essentially no serum. In one embodiment, the pluripotent human cell is cultured with the essentially serum free cell differentiation environment between approximately 1-60 days, more preferably between approximately 2-28 days, and most preferably 5-15 days.

The present invention encompasses the human neural cells and the human cell cultures enriched in neural cells produced by any of the above-described methods. In preferred embodiments, the neural cell is capable of expressing one or more of the detectable markers for tyrosine hydroxylase (TH), vesicular monamine transporter (VMAT) dopamine transporter (DAT), and aromatic amino acid decarboxylase (AADC, also known as dopa decarboxylase). In preferred embodiments, the neural cell expresses less Oct4 protein than an embryonic stem cell or a pluripotent human cell. The human neural cells or cell cultures enriched in neural cells generated using the compositions and methods of the present invention can be generated in adherent culture or as cell aggregates in suspension culture. Preferably, the human neural cells or cell cultures enriched in neural cells are produced in suspension culture. As used herein, the term “enriched” refers to a culture that contains more than 50%, 60%, 70%, 80%, 90%, or 95% of the desired cell lineage. In one embodiment, at least 80% of the human cell culture comprises neural cells. In another embodiment, the human cell culture is enriched for dopaminergic cells. In one embodiment, more than 50%, 60%, 70%, 80%, 90%, or 95% of the neural cells express tyrosine hydroxylase. In one preferred embodiment, more than 95% of the neural cells express tyrosine hydroxylase.

The human neural cells produced using the methods of the present invention have a variety of uses. In particular, the neural cells can be used as a source of nuclear material for nuclear transfer techniques, and used to produce cells, tissues or components of organs for transplant. The invention contemplates that the neural cells of the present invention are used in human cell therapy or human gene therapy to treat a patient having a neural disease or disorder, including but not limited to Parkinson's disease, Huntington's disease, lysosomal storage diseases, multiple sclerosis, memory and behavioral disorders, Alzheimer's disease, epilepsy, seizures, macular degeneration, and other retinopathies. The cells can also be used in treatment of nervous system injuries that arise from spinal cord injuries, stroke, or other neural trauma or can be used to treat neural disease and damage induced by surgery, chemotherapy, drug or alcohol abuse, environmental toxins and poisoning. The cells are also useful in treatment of peripheral neuropathy such as those neuropathies associated with injury, diabetes, autoimmune disorders or circulatory system disorders. The cells may also be used to treat diseases or disorders of the neuroendocrine system, and autonomic nervous system including the sympathetic and parasympathetic nervous system. In a preferred embodiment, a therapeutically effective amount of the neural cell or cell culture enriched in neural cells is administered to a patient with a neural disease. As used herein, the term “therapeutically effective amount” refers to that number of cells which is sufficient to at least alleviate one of the symptoms of the neural disease, disorder, nervous system injury, damage or neuropathy. In a preferred embodiment, the neural disease is Parkinson's disease.

The neural cells of the invention can also be used in testing the effect of molecules on neural differentiation or survival, in toxicity testing or in testing molecules for their effects on neural or neuronal functions. This could include screens to identify factors with specific properties affecting neural or neuronal differentiation, development, survival, plasticity or function. In this application the cell cultures could have great utility in the discovery, development and testing of new drugs and compounds that interact with and affect the biology of neural stem cells, neural progenitors or differentiated neural or neuronal cell types. The neural cells can also have great utility in studies designed to identify the cellular and molecular basis of neural development and dysfunction including but not limited to axon guidance, neurodegenerative diseases, neuronal plasticity and learning and memory. Such basic neurobiology studies may identify novel molecular components of these processes and provide novel uses for existing drugs and compounds, as well as identify new drug targets or drug candidates.

The neural cell or the human cell culture enriched in neural cells may disperse and differentiate in vivo following brain implantation. In particular, following intraventricular implantation, the cell can be capable of dispersing widely along the ventricle walls and moving to the sub-ependymal layer. The cell can be further able to move into deeper regions of the brain, including into the untreated (e.g., by injection) side of the brain into sites that include but are not limited to the thalamus, frontal cortex, caudate putamen and colliculus. In addition the neural cell or human cell culture enriched in neural cells can be injected directly into neural tissue with subsequent dispersal of the cells from the site of injection. This could include any region, nucleus, plexus, ganglion or structure of the central or peripheral nervous systems. In a preferred embodiment, following brain implantation, the neural cell or the human cell culture enriched in neural cells previously cultured with the ceramide compound induces the formation of fewer teratomas than cells or cell cultures not cultured with the compound.

The method of enriching populations of stem or progenitor cells via ceramide induced cell death has potential applications in other areas as well. For example, autologous transplants of hematopoietic stem or progenitor cells may be useful in the treatment of cancers including but not limited to cancers of the hematopoietic system such as leukemias and lymphomas as well as solid tumors. To date, this approach has had limited success due to the infusion of cancerous cells along with normal hematopoietic cells in the autologous graft (Rill, D. R., Santana V. M., Roberts W. M., 1994 Blood 84:380-383). Efforts directed at removing cancer cells from autologous grafts of hematopoietic cells by cell sorting protocols have not yet been uniformly successful in completely removing cancerous cells from the autografts resulting in the potential or actual recurrence of disease in recipients of the autologous hematopoietic graft (Dreger et al., 2000, Experimental Hematology 28:1187-1196; Rasmussen et al., 2002, Experimental Hematology 30:82-88). Incubation of hematopoietic cells with ceramide analogs or the activation of ceramide signaling pathways in these cell populations may remove cancerous or tumor forming cells within these populations.

Throughout this application, various publications are referenced. The disclosures of all of these publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments.

### EXAMPLES

### Example 1

Production of Ceramide Analogs

Ceramide analogs were produced as described in U.S. Pat. No. 6,410,597 to Bieberich, the entire contents of which are hereby incorporated by reference. Briefly, the compound S16 (N-(2-hydroxy-1-(hydroxymethyl)ethyl)-palmitoylamide) was synthesized from a solution of 50 mg (549 μmoles) of 2-amino-1,3-propanediol in 15 ml of pyridine supplemented with 1.65 mmol (457 μl) of palmitoylchloride at −30° C. The reaction mixture was stirred for 2 hours at room temperature followed by the addition of 30 ml of CH₃OH. After stirring for another 2 hours at room temperature the reaction mixture was concentrated by evaporation. For selective hydrolysis of any ester groups formed during the reaction, the concentrate was treated with a 30 ml solution of CH₃OH and sodium methoxide (pH 11-12) and stirred for 2 hours at room temperature. The reaction mixture was neutralized with dilute HCl and then concentrated. The reaction product obtained was purified by chromatography on a silica gel column (5 g) with CHCl₃/CH₃OH (5:1 by volume) as the eluent. The yield of S16 was 135 mg (75%). The purity and structure were verified by nuclear magnetic resonance (NMR) and mass spectrometry.

The octanoyl-, oleoyl-, and stearoyl derivatives (S8, S18 and SS18) were synthesized following the procedure used above for the synthesis of S16, but using octanoyl chloride, oleoyl chloride and stearoyl chloride, respectively, instead of palmitoyl chloride in the procedure.

The T16 compound was prepared by following the procedure used above for the synthesis of S16, but using bis(hydroxyethyl)amine instead of 2-amino-1,3-propanediol. The T18 was prepared by following the procedure used above for the synthesis of T16, but using oleoyl chloride instead of palmitoyl chloride in the procedure.

The ceramide compounds were lyophilized and stored in the dark until use. The compound was dissolved in ethanol to make a stock solution, and the stock solution was added to an appropriate pre-warmed tissue culture medium prior to culturing the cells with the ceramide compound.

### Example 2

Production of Essentially Serum Free MEDU Conditioned Medium, and Isolation of Bioactive Components Thereof.

Serum free MEDII (sfMEDII) was used as a source of the biologically active factor in all purification protocols. An essentially serum free MEDII conditioned medium was produced as follows. Hep G2 cells (Knowles et al., 1980 Nature 288:615-618; ATCC HB-8065) were seeded at a density of 5×10⁴ cells/cm² and cultured for three days in DMEM. Cells were washed twice with 1×PBS and once with serum free medium (DMEM containing high glucose but without phenol red, supplemented with 1 mM L-glutamine, 0.1 mM β-ME, 1×ITSS supplement (Boehringer Mannheim), 10 mM HEPES, pH 7.4 and 110 mg/L sodium pyruvate) for 2 hours. Fresh serum free medium was added at a ratio of 0.23 ml/cm² and the cells were cultured for a further 3-4 days. sfMEDII was collected, sterilized and stored. A further explanation of MEDII conditioned media can be found in International Application No. WO 99/53021.

The bioactive components of MEDII can be isolated and characterized using techniques routine to those of ordinary skill in the art. Non-limiting examples of such isolation and characterization can be found within International Application No. WO 99/53021, herein incorporated by reference in its entirety.

### Example 3

Induction of Apoptosis by Treatment of Murine ES Cells with Novel Ceramide Analogs of the β-Hydroxyalkylamine Type

Methods

In Vitro Neural Differentiation of Murine ES Cells

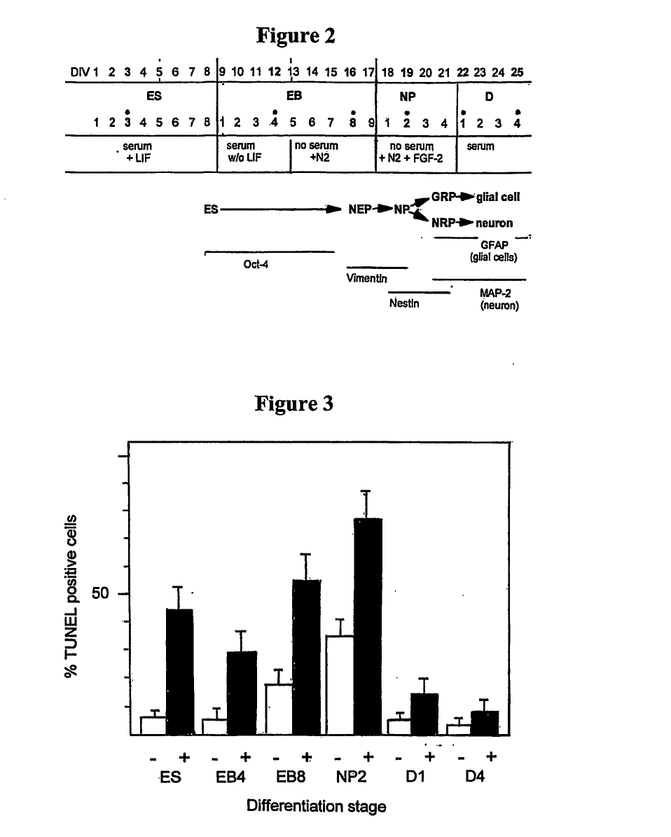

In vitro neural differentiation of mouse ES cells (ES-J1, ES-D3) followed a serum deprivation protocol as described previously (Hancock, et al., 2000, Biochem. Biophys. Res. Commun. 271: 418-421). The differentiation stages are outlined in FIG. 2. Briefly, ES cells were grown on gamma-irradiated feeder fibroblasts for four days in Knockout DMEM/15% Knockout serum replacement, supplemented with ESGRO (LIF; Chemicon; Cat No. ESG1106) at a concentration of 103 units/ml medium. ES cells were then grown for another four days on gelatin-coated bacterial culture dishes without a fibroblast feeder layer, and were then grown for three days in Knockout DMEM/15% heat-inactivated ES qualified Fetal Bovine Serum, supplemented with 10³ units LIF per ml of medium. Upon trypsinization, ES cells were transferred to bacterial culture dishes without gelatin, and embryoid body (EB) formation was induced for four days in Knockout DMEM/10% heat-inactivated ES qualified FBS without LIF (EB4 stage). On the fifth day, floating and loosely attached EBs were rinsed off and transferred to tissue culture dishes. The EBs were allowed to attach to the tissue culture dish surface by incubation for another 24 hours in Knockout DMEM with 10% heat-inactivated ES qualified fetal bovine serum. Neural differentiation due to serum deprivation was induced by cultivation of the EBs for three days in DMEM/F12 (50/50), supplemented with 1×N2 (Invitrogen/Life Technologies; Cat No. 17502, dilution of 1: 100) but without serum (EB8 stage). Serum-deprived EBs were then trypsinized, plated on poly-L-ornithine/laminin-coated tissue culture dishes and grown for four days in DMEM/F12 (50/50), supplemented with N2 and 10 ng/ml FGF-2, but without serum. his incubation period is referred to as neuroprogenitor (NP) stage due to commitment of neuroepithelial precursor cells to neuroprogenitor cells. These cells have committed during the EB stages and were expanded during the NP stage. NPs grown for 48 hours upon replating of trypsinized EBs were referred to as the NP2 stage. On the fifth day of NP formation, the medium was changed to Neurobasal (Invitrogen/Life Technologies; Cat No. 21103-049), with 5% heat-inactivated FBS, and the cells were incubated for another seven days. During this time, NPs fully differentiate to glial cells and neurons. Cells cultured for 24 hours or 96 hours upon changing the medium were referred to as the D1 or D4 stage, respectively.

ES cells were cultured and differentiated to the EB4, EB8, NP2, or D4 stage following the protocol as described above. The ceramide analog S18 was dissolved in ethanol at a concentration of 100 mM and then added to the cells at a final concentration of 75 μM in medium. The cells at the EB8 stage were incubated for 48 hours in the presence of the ceramide analog and were then transplanted into mouse brains.

Ceramide Analysis

The extraction and quantitative determination of the ceramide levels by high performance thin layer chromatography (HPTLC) followed a standard protocol as described previously (Bieberich, E., et al., 2001, J. Biol. Chem. 276: 44396-44404; and Bieberich, E., et al., 1999, J. Neurochem. 72: 1040-1049). Briefly, ES cells and ES-derived neural cultures were homogenized in 500 μl of deionized water and lipids were extracted with 5 ml of CHCl₃/CH₃OH (1:1 by volume). The lipid extract was adjusted to the composition of solvent A (CHCl₃/CH₃OH/H₂O, 30:60:8 by volume) and acidic and neutral lipids were separated by chromatography on 1 ml of DEAE-Sephadex A-25. The unbound neutral lipids were washed out with 6 ml of solvent A and were then concentrated by evaporation with a gentle stream of nitrogen. The dried residue was re-dissolved in methanol for separation by HPTLC using the running solvent CHCl₃/HOAc (methanol:acetic acid; 9:1 by volume). Lipids were stained with 3% cupric acetate in 8% phosphoric acid for quantification by comparison with various amounts of standard lipids.

Immunofluorescence Microscopy and TUNEL Assay

Differentiating ES cells were grown on cover slips and fixed with 4% paraformaldehyde in phosphate-buffered saline (PBS). Fixed cells were permeabilized with 0.5% Triton X-100 in PBS for 5 minutes at room temperature and unspecific binding sites were saturated by incubation with 3% ovalbumin in PBS for 1 hour at 37° C. The cover slips were then incubated with 5 μg/ml primary antibody (anti-ceramide clone 15B4 mouse IgM, Alexis; anti-PAR-4 rabbit IgG, Santa Cruz; anti-PCNA rabbit IgG, Santa Cruz; anti-nestin clone 401 rat IgG, BD Pharmingen) in 0.1% ovalbumin/PBS, followed by incubation with the appropriate fluorescence-labeled secondary antibody (5 μg/ml Alexa 546 conjugated anti-mouse IgG, Molecular Probes; Alexa 488 conjugated anti-rabbit IgG, Molecular Probes, Cy3 conjugated anti-mouse IgM, Jackson) for 2 hours at 37° C. The nuclei were stained by treatment with 2 μg/ml Hoechst 33258 in PBS for 30 minutes at room temperature. Apoptotic nuclei were stained using the fluorescein FragEL TUNEL assay (Oncogene) according to the manufacturer's instructions.

Statistical Analysis

Antigen specific immunostaining was quantified by counting cells that fluoresced at least twice as much as the background fluorescence. Cell counts were performed in five areas of approximately 200 cells each that were obtained from three independent immunostaining reactions. A Chi square test with one degree of freedom was applied for the statistical analysis of the distribution of two immunostained antigens. The first null hypothesis (H01) to be refuted was that the two antigens were independently distributed within the total cell population (mean of 200 cells in five counts). The expected frequency for double-staining was the frequency product for immunostaining of A or B in the total population, f(A and B)=f(A)×f(B). The second null hypothesis (H02) to be refuted was that the frequency of antigen B in the subpopulation A was identical to its frequency in the total population, f(B in A)=f(13 in A+B).

Results

The concentration of endogenous ceramide in apoptotic, undifferentiated stem cells and non-apoptotic, neural progenitor cells was determined. In contrast to cancer cells, the undifferentiated stem cells and neural progenitor cells had elevated levels of endogenous ceramide prior to treatment with the ceramide compounds, indicating that ceramide analogs of the serinol type enhance or sustain apoptosis in undifferentiated stem cells, rather than inducing or initiating apoptosis in the undifferentiated stem cells. However, neural progenitor cells, although they had elevated levels of endogenous ceramide, were protected against ceramide compound-induced and/or -enhanced apoptosis.

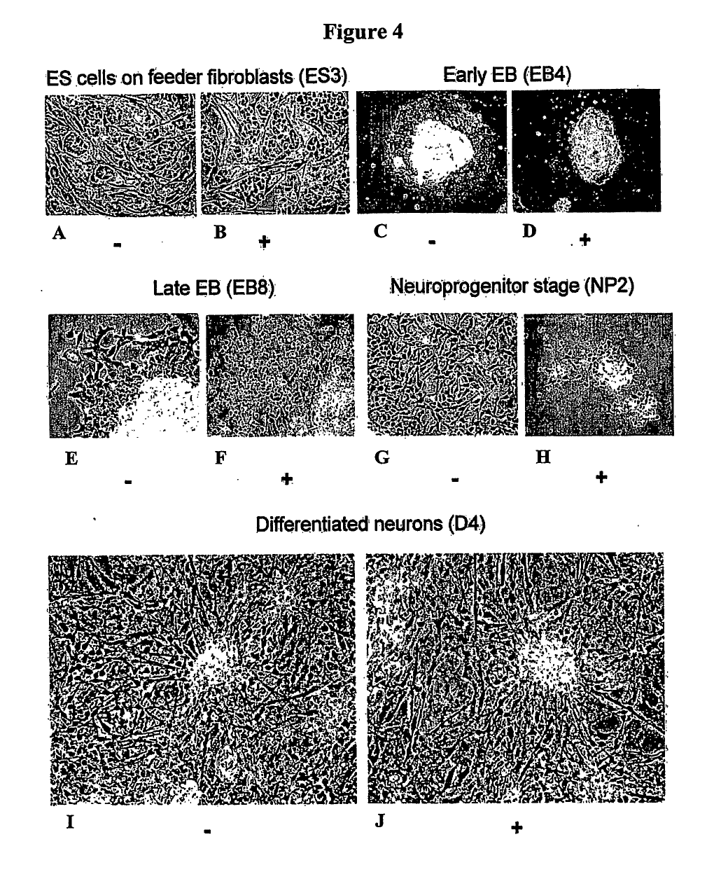

The degree of apoptosis that occurred naturally in differentiating mouse ES cells or that occurred upon incubation for 15 hours with 75 μM of the novel ceramide analogs S16 or S18, or 35 μM N-acetyl sphingosine (C2-ceramide) was determined. FIG. 2 shows the in vitro neural differentiation of mouse embryonic stem cells, indicating the various stages of differentiation. FIGS. 3 and 4 show that cell death was prominent at the EB8 or NP2 stages, whereas differentiated neurons did not reveal characteristics of apoptotic cells. The degree of apoptosis was quantified by counting TUNEL stained (apoptotic) cells. Apoptosis was elevated at the EB8 stage, when 20±5% of cells were apoptotic, and was most prominent at the NP2 stage when 35±5% of cells were apoptotic. Incubation with S16, S18, or C2-ceramide enhanced apoptosis, and increased the number of TUNEL stained cells to 45±10% at the EB8 stage and 70±10% at the NP2 stage. Enhancement of apoptosis by ceramide analogs was also observed in undifferentiated ES cells, where 40±10% of cells were apoptotic, and at the EB4 stage, where 25±5% of cells were apoptotic.

The sensitivity of differentiating NP cells rapidly decreased upon the post-treatment plating of trypsinized EBs at day 8 (EB8). Sensitivity to ceramide analogs was highest for NP2, while the sensitivity to the analogs was already less than 20% at the D1 stage. TUNEL staining revealed that differentiated neurons at the D4 stage did not show significant levels of apoptosis (<10±5%) upon incubation with ceramide analogs.

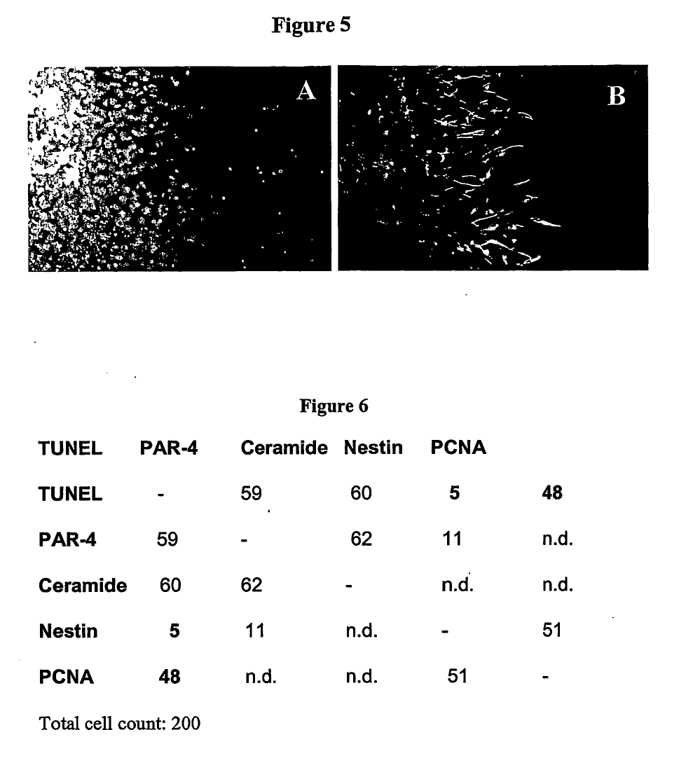

FIG. 4F shows that at the EB8 stage, a rim of cells surrounding the central embryoid body resisted apoptosis induced by novel ceramide analogs. Immunostaining of EBs with an antibody against nestin, a marker protein for neural progenitor cells, revealed that this rim of non-apoptotic cells strongly stains for nestin (FIG. 5B). Therefore, neural progenitor cells that express nestin were less sensitive toward ceramide induced or -enhanced apoptosis, whereas nestin-negative, undifferentiated cells were sensitive to ceramide-enhanceable apoptosis. Cell counts revealed that of TUNEL positive cells, 8% were nestin positive (5/65) while 80% (108/135) of the TUNEL negative cells expressed nestin protein.

A quantitative determination of different marker proteins and TUNEL staining for apoptotic cells showed that predominantly nestin negative, proliferating cell nuclear antigen (PCNA) positive cells underwent apoptosis (FIG. 6). PCNA is a specific marker protein for cells that undergo rapid cell division. PCNA positive cells are not neural progenitor cells, but show rapid proliferation. These highly proliferative cells are likely to be residual pluripotent stem cells since these cells are known to have a cell cycle with greatly abbreviated G1 and G2 phases while differentiated cells derived from pluripotent stem cells have longer cell cycles with longer G1 and G2 phases (WO 01/23531, herein incorporated by reference in its entirety). The elimination of these rapidly proliferating cells by selective apoptosis will thus reduce significantly the risk of teratoma formation after transplantation of pluripotent stem cell-derived cells into the host tissue.

### Example 4

Injection of Ceramide Analog Treated EB-Derived Stem Cells into Mouse Brains

Methods

In vitro differentiating ES cells at stage EB8 were incubated for 24-48 hours with 75 μM S18, or 35 μM N-acyl sphingosine or other ceramide analogs. Protein was isolated from cells incubated with S18 for 24 hours and from untreated cells, separated by SDS-PAGE, and the expression of Oct4 was analyzed by immunoblotting.

Prior to injection into the mouse brain, ES cells were labeled with Vybrant-DiI (rhodamine fluorescence) for permanent vital staining and were mixed with India ink in order to track the injection channel and cell migration/tissue integration. 1×10⁴ of the untreated ES-J1 cells were injected, while 2×10⁴ of the S18-treated cells were injected in order to control for the percentage of cells lost to apoptosis. The ES cells were injected into the right brain hemisphere (bregma −1.5 mm, 1 mm lateral of central suture, 2.0 mm deep) of 8-10 day old C57BL6 mice using a Hamilton syringe. After 7-21 days, the mice were sacrificed, the brain isolated and fixed with 10% PBS-buffered formalin. The brains were Vibratome sectioned at 100 μm. The distribution of the injected cells was determined by fluorescence microscopy.

Results

The protein preparation from S18 treated cells demonstrated only 25% of the Oct4 immunostaining found in the untreated control cells. This indicated that Oct4 protein levels were suppressed, or that Oct4 expressing cells were eliminated such that a 75% decrease in Oct4 protein levels was observed after treatment with S18.

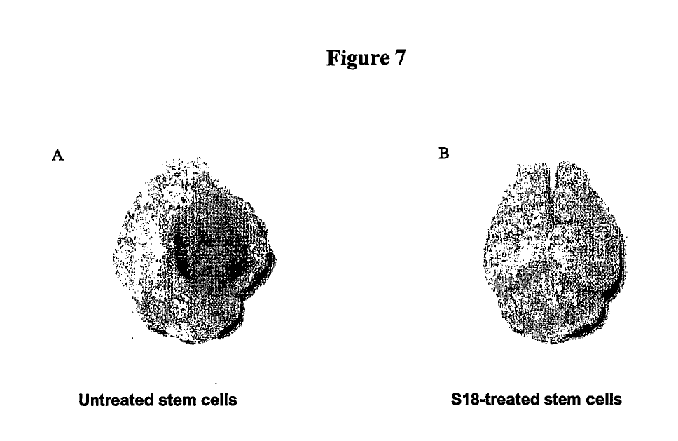

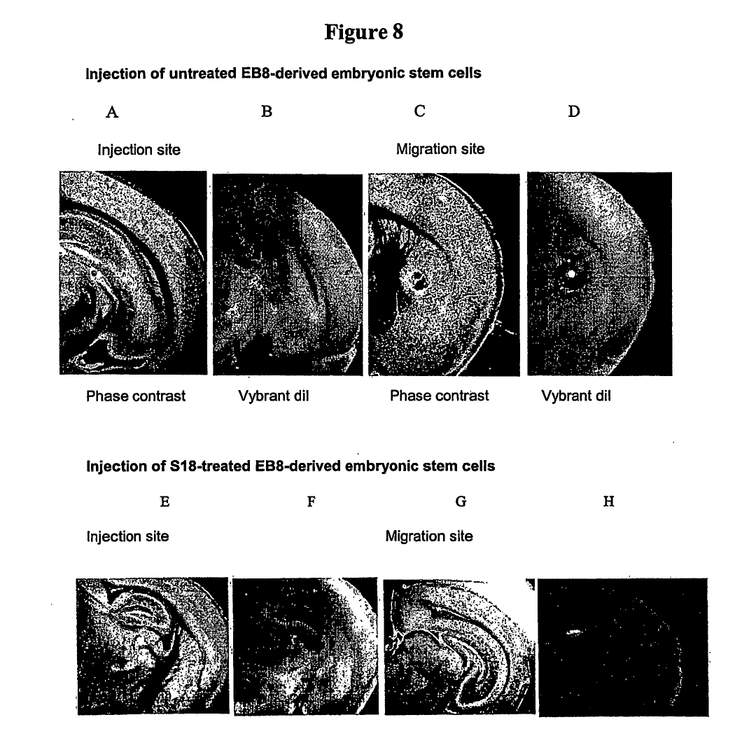

FIG. 7A shows that ten days after injection of the cells, massive teratoma formation was found on the right side of the brain that was injected with untreated, control cells. However, EB8-derived cells that were treated with S18 did not show teratoma formation (FIG. 7B). In another experiment, EB8-derived cells were stained with a fluorescent marker dye, Vybrant diI, in order to track the migration and integration of the injected cells into the recipient's brain tissue. FIGS. 8A-D show that untreated cells formed numerous teratomas that resulted in death of the recipient at 8 days post-injection. S18-treated EB8-derived cells, however, did not form teratomas, migrated to the hippocampus, and integrated into the host's brain tissue (FIGS. 8E-H). The host injected with the ceramide analog treated cells was killed after 21 days in order to analyze the brain tissue. From two separate transplantation experiments a total of 5 animals were implanted with S18 treated cells. No teratomas were detected in the animals implanted with S18 treated cells. A total of 4 control animals were implanted with untreated cells. One of these controls died and its brain could not be analyzed, the remaining three control animals all contained teratomas formed from the injected untreated cells.

### Example 5

Induction of Apoptosis in Mouse Neuroblastoma Cells

Mouse neuroblastoma (F-11) cells were incubated for 24 hours in 0.1, 0.2, 0.5, or 1.0 μM of laurylgallate (Aldrich). Apoptosis was determined by punctate staining of condensed nuclei with Hoechst 33258 (Sigma, 2 μ/ml medium for 30 minutes at room temperature).

Results

At a concentration of 0.5 μM laurylgallate, 50% of the neuroblastoma cells were observed to undergo apoptosis. At 1.0 μM of laurylgallate, 100% of the cells had undergone apoptosis. These results indicates that laurylgallate is a very potent inducer of apoptosis in neuroblastoma cells, and likely will enhance apoptosis is undifferentiated ES cells as well.

### Example 6

Cell Culture Conditions for Human Embryonic Stem Cells

Manual Passaging of Human ES Cells

Human embryonic stem cells (HESCs) identified as BGN01 (BresaGen, Inc. Athens, Ga.) were used in this work. The HESCs were grown in DMEM/F12 (50/50) supplemented with 15% FCS, 5% knockout serum replacer (Invitrogen), 1× non-essential amino acids (NEAA; Invitrogen), L-Glutamine (20 mM), penicillin (0.5 U/ml), streptomycin (0.5 U/ml), human LIF (10 ng/ml, Chemicon) and FGF-2 (4 ng/ml, Sigma). The human ES cells were grown on feeder layers of mouse primary embryonic fibroblasts (MEFs) that were mitotically inactivated by treatment with mitomycin-C. Feeder cells were re-plated at 1.2×10⁶ cells per 35 mm dish. The mitotically inactivated fibroblasts were cultured for at least 2 days prior to the plating of HESCs. Alternatively, HESCs were grown on 2×10⁶ MEFs per 35 mm dish, where the medium contains 20% KSR, and the is pre-conditioned on MEF feeders for 24 hours prior to plating the HESCs.

The HESCS were manually passaged onto fresh fibroblast feeder layers every 3-4 days using a fire-pulled Pasteur pipette. Briefly, the barrel of the Pasteur pipette was melted solid and drawn out to a solid needle approximately 1 cm long and approximately 25 μm in diameter, which was sequentially pressed through HESC colonies to form a uniform grid of cuts. The same needle was passed under the colonies to lift them from the feeder layer. Entire plates of HESCs were harvested, then the colonies were broken into individual pieces defined by the grid by gentle pipetting using a 5 ml serological pipette. The pieces from a single plate were split between 2 or 3 new plates that were coated with feeder layers of mitotically inactivated mouse primary embryonic fibroblasts.

SSEA4 Selection and Bulk Passaging of HESCs

SSEA4 staining appears to be closely associated with the undifferentiated state of HESCs. Undifferentiated domed HESC colonies show a uniform distribution of SSEA4 immunostaining, while differentiating HESC colonies show reduced or no expression of SSEA4 in morphologically differentiated cells. An example of this is the reduced SSEA4 expression in morphologically differentiated cells that occurs within the crater cells located in the center of manually passaged HESCs that are plated onto fresh feeder layers These crater cells grow as a monolayer, surrounded by multilayered morphologically undifferentiated HESCs. Since SSEA4 appears to be selective for a population of undifferentiated HESCs, it was chosen to use as a selectable marker.

Undifferentiated HESCs were selected by magnetic sorting using an anti-SSEA4 antibody (Developmental Studies Hybridoma Bank) and the MACS separation system (Miltenyi Biotec) according to the manufacturers instructions. Briefly, manually passaged BESCs were harvested by treating with 1 mg/ml Collagenase (Gibco) for 5 minutes, followed by treating with 0.05% Trypsin/EDTA for 30 seconds. Colonies were then flushed off the top of the feeder layer and dissociated to an essentially single cell suspension, leaving the feeder cells behind as a net. The trypsin was neutralized with 10% FBS/10% KSR human ES medium and passed through a cell strainer (Becton Dickinson). For blocking, cells were pelleted and resuspended in staining buffer (5% FBS, 1 mM EDTA, penicillin (0.5 U/ml) and streptomycin (0.5 U/ml), in Ca²⁺/Mg²⁺ free PBS).

The cells were pelleted and resuspended in 1 ml primary anti-SSEA4 antibody diluted 1:10 in staining buffer, and incubated at 4° C. for 15 minutes. 9 ml staining buffer was then added and the cells were pelleted, washed with 10 ml staining buffer and re-pelleted. 1×10⁷ cells were resuspended in 80 μl staining buffer and 20 μl magnetic goat anti-mouse IgG MicroBeads were added, mixed and incubated at 4° C. for 10 minutes. The volume was then brought to 2 ml with staining buffer and 2 μl of a fluorescent conjugated secondary antibody (Alexa-488 conjugated goat anti-mouse IgG, Molecular Probes) was added to enable fluorescent analysis of the separation. The sample was incubated for 5 minutes at 4° C., then the volume was brought to 10 ml with staining buffer and the cells were pelleted and washed in 10 ml staining buffer and re-pelleted. The cells were resuspended in 500 μl staining buffer and applied to a separation column that had been prepared by washing it three times with 500 μl staining buffer. The column was positioned on the selection magnet prior to application of the cells and the flow-through and three washes with 500 μl staining buffer were collected. These cells in these fractions were presumably a SSEA4 negative population. The column was removed from the magnet, 500 μl staining buffer was added and forced through with a plunger, and the presumed SSEA4 positive cell population was collected in a 15 ml tube. 20% KSR human ES growth medium was added to bring the volume to 10 ml, and the cells were pelleted and resuspended in 1 ml of the same medium. 10⁵ SSEA4 selected HESCs were plated on 35 mm dishes plated with a mouse embryonic fibroblast feeder layer, and the cells were maintained and passaged in 20% KSR growth medium (see below).

To examine the effectiveness of the selection, aliquots of the flow/wash sample and SSEA4 selected sample were analyzed by fluorescence microscopy. Approximately 75% of the cells from the retained fraction were SSEA4 positive, indicating effective enrichment.

Bulk passaged HESCs were grown in DMEM/F12 (50/50) supplemented with 20% knockout serum replacer (KSR; Invitrogen), 1×NEAA (Invitrogen), L-Glutamine (20 mM), penicillin (0.5 U/ml), streptomycin (0.5 U/ml), human LIF (10 ng/ml, Chemicon) and FGF-2 (4 ng/ml, Sigma). For passaging, cells were treated with 1 mg/ml Collagenase (Gibco) for 5 minutes, followed by 0.05% Trypsin for 30 seconds and the cells were then dissociated with a 1 ml pipette. The feeder layer remained as a mesh and was removed with a pipette. DMEM/F12 (50/50) supplemented with 10% FCS and 10% KSR was added to the HESC suspension, followed by centrifugation, aspiration and resuspension in culture medium. HESCs were replated at 1×10⁵ cells per 35 mm dish on a feeder layer.

Generation of Embryoid Bodies from Cells in the Crater of an ES Colony

The colony morphology of HESCs was observed to differ from the typically observed multilayered, domed colonies when HESCs were plated onto feeder cells that had been freshly plated. When HESC's were plated on feeder cells that were 0-6 hours old, but not on feeders that were 2 days old or older, typical HESC colonies formed except that in the central region of the colony a “crater” was observed. Domed colonies were observed when HESCs were plated onto feeders that were at least 2 days old. These central or crater cells formed a monolayer of uniform cells within a ring of multilayered HESCs. This monolayer was in direct contact with the tissue culture plastic, or the extracellular matrix that was left behind as the HESC colony had pushed out the underlying feeder layer. HESC colonies typically displace the underlying feeder layer as they seed and proliferate. Cells within the crater expressed the pluripotent marker Oct4, although apparently at a reduced level compared to the surrounding ring of HESCs, indicating that they are a novel, partially differentiated cell type derived from the HESCs. This approach allowing the controlled development of crater HESC colonies occurred within 3 to 5 days and generated a uniform monolayer of central cells, as opposed to stochastic differentiation proceeding over several weeks and leading to a complex heterogeneous culture (Reubinoff et al., 2001 Nature Biotech 19, 1134-1140).

Domed colonies were preferred for continual passaging, while monolayer cultures were preferred for generating serum free embryoid bodies.

Formation of Essentially Serum Free Embryoid Bodies

Manually passaged HESC cultures were washed once with DMEM/F12 and once with DMEM/F12 supplemented with 1×N2 supplement (Invitrogen). Undifferentiated HESC colonies were harvested into uniform colony pieces of approximately 10-100 cells using the manual passaging methods described above. Pieces were transferred to 15 ml tubes and washed in 10 ml DMEM/F12 plus 1×N2 supplement. The pieces were left to settle, and the medium was aspirated. The pieces were resuspended in 2.5 ml of medium, and transferred to suspension dishes.

Suspension dishes were prepared by coating the surface of non-tissue culture plastic Petri dishes with a layer of agarose. The agarose coating was generated by pouring a molten solution of 0.5% agarose in DMEM/F12 medium into the Petri plates. The agarose coating was equilibrated in DMEM/F12 medium. Suspension cultures contained 2.5 ml of medium for 35 mm dishes, or 10 ml of medium for 100 mm dishes.

Essentially serum free embryoid bodies were cultured in suspension for up to four weeks, with replenishment of the medium every 3-4 days. The essentially serum free embryoid bodies were passaged every 5-7 days by cutting them into pieces with drawn out solid glass needles. At passaging, the embryoid bodies contained approximately 5000-10,000 cells and were divided into 4-10 pieces. Essentially serum free embryoid bodies formed in the presence of DMEM/F12 with 1×N2 and 4 ng/ml FGF-2 were termed sfEBs, while essentially serum free embryoid bodies formed in the presence of DMEM/F12 with 1×N2, 4 ng/ml FGF-2 and 50% MEDII were termed sfEBMs.

Essentially serum free embryoid bodies were generated from HESC crater cells by removing the feeder layer and HESCs growing on their surface. Watchmaker's forceps were used to hold the feeder layer at the side of the culture dish, and lifted this layer and the attached multilayered HESC from the dish. This manipulation peeled the feeder layer and the multilayered parts of the HESC colonies off of the dish and the monolayer crater cells were left attached to the dish. Glass needles were used to cut the crater monolayer to 50-200 cell size pieces, and lift them from the dish. These pieces were grown in suspension culture in the same serum free conditions as above (DMEM/F12, 1×N2, L-Glutamine (20 mM), penicillin (0.5 U/ml), streptomycin (0.5 U/ml), 4 ng/ml FGF-2, with or without 50% MEDII).

Essentially serum free embryoid bodies were generated from SSEA4 selected monolayer HESC colonies by Collagenase treatment. HESC cultures were treated with protease, and then washed with DMEM/F12 1×N2 and 4 ng/ml FGF2. The monolayer colonies remained attached to the tissue culture plastic but became less tightly associated with the feeder layer. The feeder layer was removed using watchmaker's forceps as above. The monolayer HESC colonies were scraped off the dish using a glass needle, were transferred to a 15 ml tube and washed twice with the same medium and centrifuged (1000 rpm, 4 minutes). The HESC colonies were transferred to suspension dishes for development as essentially serum free embryoid bodies grown in the conditions described above (DMEM/F12, 1×N2, L-Glutamine (20 mM), penicillin (0.5 U/ml), streptomycin (0.5 U/ml), 4 ng/ml FGF-2, with or without 50% MEDII).

Immunostaining

For immunostaining, seeded embryoid bodies were rinsed with 1×PBS and fixed in 4% paraformaldehyde, 4% sucrose in 1×PBS for 30 minutes at 4° C. The cells were then washed in 1×PBS and stored at 4° C. Essentially serum free embryoid bodies in suspension were disaggregated and attached to a glass slide using a standard cytospin approach for immunostaining (Watson P. A., J. Lab. Clin. Med. 68:494-501, 1966). sfEBMs were washed with 1×PBS and disaggregated with 0.05% trypsin and gentle trituration. The cell suspension was washed with culture medium, pelleted and resuspended in HESC medium and 1×104 cells were attached to a glass microscope slide by centrifugation at 300 g for 4 minutes using a cytospin apparatus (Heraeus Instruments GmbH). The attached cells were fixed immediately with 4% paraformaldehyde, and 4% sucrose in 1×PBS for 15 minutes, followed by three separate 5-minute washes in 1×PBS. Alternatively, the embryoid bodies were not attached to slides as cytospins, and were studied by whole mount immunostaining.

To perform immunostaining on fixed whole mount samples, cells or cytospins, the samples were washed in block buffer (3% goat serum, 1% polyvinyl Pyrolidone, 0.3% Triton X-100 in wash buffer) for 30 minutes, and then incubated with the appropriate dilution of the primary antibody, or combination of antibodies for 4-6 hours at room temperature. The primary antibodies were anti-Map2, a mouse monoclonal antibody recognizing the Map-2 a, b and c isoforms (Sigma, Catalog # M4403) at a 1/500 dilution; anti-Nestin, a rabbit polyclonal antibody (Chemicon, Catalog # AB5922) at a 1/200 dilution; anti-Oct4, a rabbit polyclonal antibody (Santa Cruz, Catalog # sc-9081) at a 1/200 dilution; sheep anti-Tyrosine Hydroxlyase (TH) antibody (Pel-Freez, Catalog # P60101-0) at a 1/500 dilution; anti-phosphoHistoneH3, a rabbit polyclonal antibody (Upstate, Catalog # 06-570) at a 1/400 dilution; anti-SSEA4, a mouse monoclonal antibody (Developmental Studies Hybridoma Bank, Catalog # MC-813-70) at a 1/5 dilution. The cells were then washed in wash buffer (50 mM Tris-HCL pH 7.5, and 2.5 mM NaCl) 3 times for 5 minutes each wash. The cells were then incubated for a minimum of 2 hours in secondary antibodies diluted 1:1000, followed by washing in wash buffer. The secondary antibodies were appropriate combinations of Alexa-350 (blue), 488 (green) or -568 (red) conjugated goat anti-chicken, anti-rabbit, anti-sheep or anti-mouse antibodies, all available from Molecular Probes. Some samples were stained with 5 ng/ml DAPI to detect cell nuclei, and were then washed from overnight to 2 days in a large volume of wash buffer. The slides were mounted with mounting medium and a cover slip. Slides were visualized using either a NIKON TS100 inverted microscope or a NIKON TE 2000-S inverted microscope with a Q Imaging digital camera.

### Example 7

Neural Differentiation of Essentially Serum Free Embryoid Bodies

HESCs were grown in suspension as embryoid bodies in essentially serum free conditions in the presence of 50% conditioned medium from the HepG2 hepatocarcinoma cell line (MEDII conditioned medium). The sfEBMs were cultured in suspension for up to 6 weeks, with passaging every 10 to 15 days. Passaging was performed by using glass needles to dissect the EBs into pieces, paying particular attention to the isolation of structured rosette regions. Non-rosette regions were generally removed from the culture during the passaging process, although the solid material could regenerate prior to the next passage.

Structured regions from essentially serum free embryoid bodies were seeded onto polyornithine and laminin coated permanox slides for adherent culture and further analysis. Essentially serum free embryoid bodies (sfEBs and sfEBMs) were cut into pieces using glass needles and 1-15 pieces were plated onto polyornithine/laminin coated permanox chamber slides in the same medium used for suspension culture. Polyornithine/laminin coated slides were prepared by diluting polyornithine to 20 μg/ml in tissue culture grade water, coating chamber wells at 37° C. overnight, washing the wells twice with water and coating the chamber wells with 1 μg/ml laminin at 37° C. for 2 hours to overnight. The slides were washed with water and 1×PBS prior to plating the cells. The embryoid bodies were cultured on these slides for 2-7 days.

Results

The structured rosette regions that were first observed morphologically between 7-10 days after derivation are neurectoderm/neural precursor/neural tube cell types. The rosette regions could comprise more than 50% of the mass of an essentially sfEBM. These structures take the form of spherical rosettes with a distinct radial appearance and central cavity surrounded by a ring of cells that is 4-8 cells in width. Other morphologically distinct regions that were observed in essentially serum free embryoid bodies included fluid filled cysts and homogeneous solid regions. Immunostaining of sections and cytospins demonstrated the presence of neurons (Map2+ cells) in sfEBMs in suspension. The neuronal networks were intermingled with, and surrounded the rosette structures. When seeded in adherent culture, rosettes grew as circular or ovoid radial structures and were surrounded by large interconnected mats of neurons that included many presumptive dopaminergic neurons that stained positively for TH.

In addition, Real Time PCR analysis of neural precursor (Sox1), pan-neuronal (map2) markers and dopaminergic transcription factor markers (En1, Nurr1, Pitx3 and Lmx1b) was performed. The normalized serum free/serum expression ratio was determined using the REST software. Lmx1b was analyzed by end point PCR and GAPDH-normalized expression ratio calculated by densitometry. It was noted that Sox1, En1, Nurr1, Pitx3 and Lmx1B were all upregulated in cells formed in essentially serum free conditions in comparison to cells formed in serum containing conditions.

| | | SERUM | | SERUM FREE | | | |
|---|---|---|---|---|---|---|---|
| | EFF | Mean CP | SD | Mean CP | SD | Ratio | p value |
| GAPDH | 1.49 | 17.018 | 0.09 | 16.839 | 0.26 | 1.07 | 0.706 |
| SOX1 | 1.47 | 27.745 | 0.01 | 23.287 | 0.04 | 5.188 | 0.001* |
| MAP2 | 1.72 | 19.091 | 0.17 | 22.369 | 0.21 | 0.206 | 0.001* |
| EN1 | 1.61 | 31.397 | 0.14 | 28.104 | 0.01 | 5.839 | 0.081 |
| NURR1 | 1.91 | 24.248 | 0.05 | 23.244 | 0.03 | 1.783 | 0.025* |
| PITX3 | 1.62 | 32.512 | 0.08 | 30.750 | 0.06 | 2.179 | 0.001* |
| LMX1B^{¶} | | | | | | 1.5 | |

### Example 8

Reduction in the Level of Oct4 Protein in Differentiated HESCs

The Oct4 transcription factor is a tightly regulated marker of pluripotency in the mouse, and expression of Oct4 mRNA in human inner cell mass and ES cultures has been confirmed (Hansis et al., 2000, Mol. Hum. Reprod. 6(11), 999-1004, and Reubinoff et al., Nature Biotech. 2000, 18, 399-404). However, the restriction of Oct4 protein to pluripotent cells in humans has not been examined thoroughly. Manually passaged HESC cultures containing domed or cratered colonies were stained with anti-Oct4 antibodies.

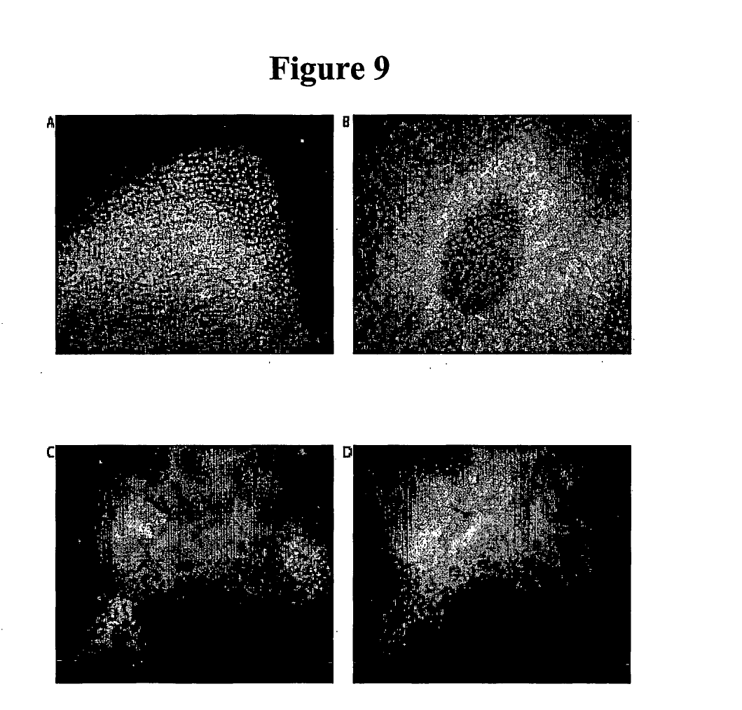

It was observed that the Oct4 protein is expressed at high levels in undifferentiated HESCs (FIG. 9A) and that levels of the Oct4 protein are down-regulated following differentiation (FIG. 9B). An unexpected characteristic of immunostaining in the culture systems analyzed was that differentiated human cells retained a reduced but detectable level of Oct4. However, when seeded sfEBM cultures were fixed and immunostained, a process that maintains the morphology of a culture, the difference between the two types of Oct4 expression was clearly distinguishable. High level Oct4 expression was only observed as bright nuclear staining in tightly packed but evenly spaced cells. Therefore immunostaining for Oct4 expression during neural differentiation in embryoid bodies was a suitable assay for the presence of residual compartments of pluripotent cells.

To monitor the persistence of pluripotent cells during sfEBM differentiation, essentially serum free embryoid bodies were generated from domed HESC colonies or monolayer crater ES cells. The sfEBMs were grown in suspension for 3-7 days, seeded onto polyornithine/laminin coated chamber slides, cultured for 3-5 days in the same medium and fixed for immunostaining. The presence of residual nests of pluripotent cells was demonstrated by clusters of high level Oct4 immunostaining amongst the generalized low level of Oct4 staining seen in the neuralized culture (FIG. 9C). The Oct4 immunoreactivity was nuclear-specific. High level Oct4 expression was not associated with the neural rosettes, which were visualized by the characteristic radial pattern of nuclei stained with DAPI (FIG. 9D). The presence of nests of residual pluripotent cells was still observed in sfEBMs that were cultured for over one month, with several passages specifically attempting to purify the neural rosette material, highlighting the persistent nature of these pluripotent cells and their implied teratoma forming potential when transplanted.

### Example 9

Induction of Apoptosis by S18 Treatment of Seeded Embryoid Bodies

Treatment of EBs with S18

sfEBMs were derived from domed HESC colonies, grown in suspension for 24 days with one passage, and seeded to polyornithine/laminin coated chamber slides in DMEM/F12, supplemented with 1×N2 (Gibco), and 1% FCS. The seeded sfEBMs were treated with 6, 8 or 10 μM S18 dissolved in the media for 36 hours. The cultures were then washed with DMEM/F12, supplemented with 1×N2, and 4 ng/ml FGF-2 and incubated for 24 hours in 50% DMEM/F12, 50% MEDII, supplemented with 1×N2, and 4 ng/ml FGF-2 before fixing and staining with DAPI.

Apoptosis in seeded serum free embryoid bodies was monitored by morphological observation of cell death and DAPI staining to reveal apoptotic nuclei. Apoptotic nuclei were observed as obviously fragmented and degenerating nuclei, with small punctuate patterns of DAPI staining. Rosette regions from essentially serum free embryoid bodies in suspension were passaged further in the same medium, either withdrawing S18 or culturing the embryoid bodies for an additional 4 to 8 days in the presence of S18. Rosette regions were then seeded onto polyornithine/laminin coated slides for analysis of proliferation and differentiation to neural lineages.

Results

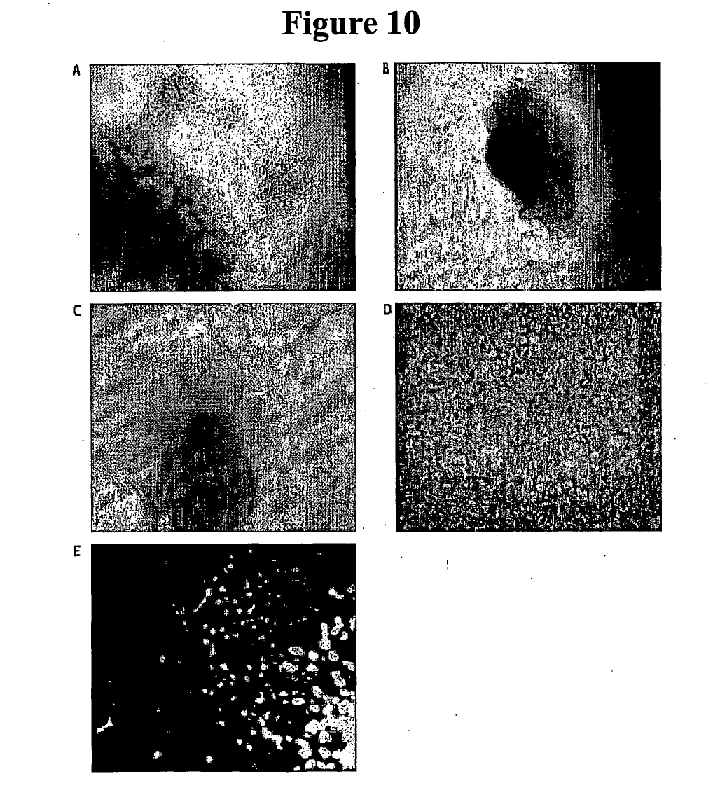

Prior to S18 treatment, the seeded cultures were heterogeneous and contained extensive neural rosette structures (FIG. 10A) as well as other cell types, such as presumptive glial cells, or other unidentified cell types. S18 treatment induced apoptosis of a large proportion of the culture at each dosage, and this effect was observed within 24 hours of treatment (FIGS. 10B, and 10C). No differences were observed between the different doses of S18. Overall, the general morphology of the culture was significantly affected, with a high level of cell death. The level of cell death is dependent upon the proportion of cell rosettes at the time of treatment. This proportion will vary, as will the level of cell death. Cellular debris was observed surrounding the seeded sfEBM, indicating that the cell types that had proliferated away from the sfEBM were killed. Neural rosette structures did not appear to be adversely affected by the S18 treatment, indicating that they were resistant to the induction of apoptosis mediated by this ceramide analog. Morphologically normal rosettes could be observed within an otherwise generally apoptotic culture (FIGS. 10C, and 10D). DAPI staining of cultures 24 hours after S18 withdrawal demonstrated that rosette cells had maintained morphologically normal nuclei, whereas cells on the periphery of the culture exhibited condensed nuclei, a characteristic of apoptotic cells (Kerr, Wyllie and Currie, 1972. Cancer 26: 239-257; FIG. 10E). The possibility that non-rosette cells in the multilayered region of the seeded sfEBM survived S18 treatment could not be addressed by this analysis. The observation that morphologically normal nuclei were an indicator of viable cells was strengthened by the observation of mitotic figures with DAPI staining, 24 hours after S18 withdrawal. This result indicated that the cells that survived treatment with S18 were capable of proliferation.

In summary, the S18 ceramide analog appeared to induce apoptosis efficiently in a range of different cell types in seeded serum free embryoid bodies, and this induction appeared to be selective, with neural rosette cells appearing not to be affected. The application of S18 to embryoid bodies thus provided a population of neural rosette cells with high purity.

### Example 10

Ceramide Analog S18 Treatment of Essentially Serum Free Embryoid Bodies in Suspension

Essentially serum free embryoid bodies (sfEBMs) were generated as described in Example 6, and were exposed to S18 at different stages of their development in order to assess the timing of depletion of high Oct4 expressing cells, and in order to determine when neural rosettes could be selected. The sfEBMs in suspension were treated with 10 μM S18 in 50% DMEM/F12, 50% MEDII, supplemented with 1×N2, Glutamine (20 mM), penicillin (0.5 U/ml), and streptomycin (0.5 U/ml) for varying amounts of time, and the sfEBMs were then evaluated histologically and by immunocytochemistry.

Essentially serum free embryoid bodies were derived from protease passaged cells and grown in the presence of 50% MEDII conditioned medium. The embryoid bodies were exposed to 10 μM S18 in the same medium from day 6 to 9 after derivation. At day 9 the S18 treated sfEBMs and matched control sfEBMs not exposed to S18 were fixed, embedded in plastic, cut to 3 micron sections and stained with DAPI to enable the precise determination of the proportion of the total healthy nuclei of an sfEBM that were rosette cell nuclei.

Results

It was not possible to derive sfEBMs from monolayer crater cells in the presence of 10 μM S18. No viable embryoid bodies were observed in the suspension culture after four days of S18 treatment, indicating that cells resistant to the induction of apoptosis were not present at this stage of the culture.

Conversely, sfEBMs at day 14 exhibited extensive neural rosette structures. This material was exposed to 10 μM S18 in 50% MEDII medium for 2 days, followed by manual passaging, and an additional 4 days in 10 μM S18 in the same medium. While 48 hours exposure to S18 did not have overt morphological effects on the sfEBM, when the embryoid bodies were manually passaged it was apparent that there was extensive apoptosis in the bodies. The non-rosette regions of the sfEBM fragmented when manipulated and released extensive stringy material that was indicative of genomic DNA from lysed cells. However, the rosette regions were morphologically normal and could be separated from all other degenerate regions of the sfEBM. The rosette pieces were incubated in 10 μM S18 for a further 4 days, and the medium was then switched to 50% DMEM/F12, 50% MEDII, supplemented with 1×N2, and 4 ng/ml FGF-2 at day 20 after the initial derivation of the embryoid bodies.

At day 21 some ceramide selected sfEBMs were seeded onto polyornithine/laminin coated slides, cultured in the same medium for an additional 8 days, and fixed for immunostaining. These seeded pieces developed as rosette cultures and mats of neurons were observed differentiating from these precursors.

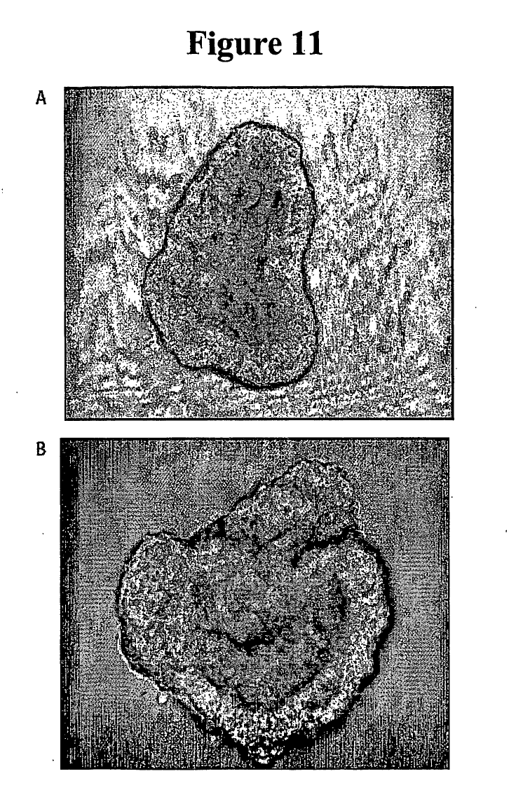

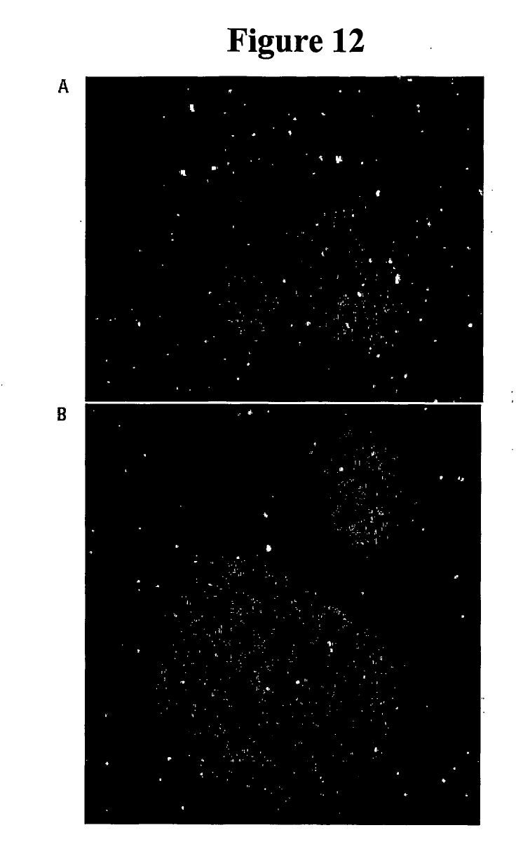

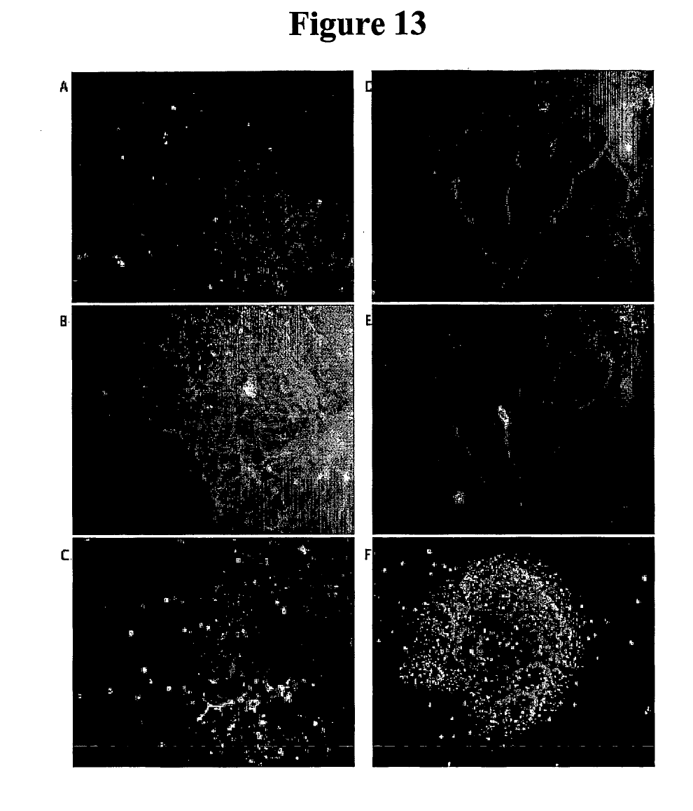

Other ceramide selected sfEBMs were maintained in suspension, and were cultured for an additional 25 days, until 45 days after their initial derivation. These suspension cultures were passaged once during this time and initially proliferated at a rate similar to seeded neural rosettes, although their growth rate slowed after around day 40 after initial derivation. At day 35, the selected sfEBMs in suspension consisted of what appeared to be essentially pure neural rosette material, without any obvious regions comprised of different cell types (FIGS. 11A and 11B).

The S18 selected sfEBM that were seeded at day 21 were analyzed by immunocytochemistry with antibodies directed against Oct4, Map2, TH and phospho-Histone H3. Staining with anti-Oct4 indicated that no regions of high Oct4 expression could be detected in any of the S18 treated samples (FIGS. 12A and 12B), indicating that no residual nests of pluripotent cells survived exposure to S18. The same result was seen in additional experiments when sfEBMs were generated and treated with 10 μM S18 in suspension prior to plating. Low level Oct4 expression was detected in rosettes (FIGS. 12A, 12B; FIG. 13A) and other cell types that were present in the cultures. While these cultures had a high proportion of rosette cells, it was clear that other cell types were present, such as neurons, as well as other presumed neuralized cell types derived from the rosette precursor cells. Immunostaining with anti-Map2 (FIGS. 13B, and 13D), which recognizes a microtubule associated protein in the dendrites of mature neurons, demonstrated the presence of networks of differentiated neurons associated with neural rosettes. Staining with anti-TH, which recognizes tyrosine hydroxylase, the rate limiting enzyme in dopamine biosynthesis, demonstrated that presumptive dopaminergic neurons or their precursors were not ablated by exposure to 10 μM S18 (FIGS. 13C, and 13E). The histone H3 protein is phosphorylated during mitosis and is an effective marker of mitotic cells.

Seeded S18 selected sfEBMs were stained with anti-phosphoHistone H3 and DAPI (FIG. 13F). The presence of neural rosettes was indicated by their characteristic radial pattern. PhosphoHistone H3 expression demonstrated that these cultures were actively proliferating at the time they were fixed (day 28 after derivation, 8 days after withdrawal of S18). PhosphoHistone H3 staining within the neural rosettes indicated that these precursor cells were still mitotically active after exposure to S18 and could therefore be expanded further.

### Example 11

SSEA4 Selection and Protease Passaging Techniques Generate a Homogeneous Cell Population from ES Cells

Methods

Embryoid bodies were generated from SSEA4 selected and bulk passaged cells as described in Example 6.

Immunostaining

Immunostaining was performed as described in Example 6 for nestin and Oct4.

For immunostaining with SSEA1, SSEA3, SSEA4, Tra1-60, and Tra1-81, samples were washed in block buffer (3% goat serum, 1% PVP in PBS) for 30 minutes, and then were incubated with the appropriated dilution of the primary antibody, or combination of antibodies for 4-6 hours at room temperature. The primary antibodies used were anti-SSEA1, a mouse IgM antibody (Developmental Studies Hybridoma Bank, Catalog # MC-480), undiluted; anti-SSEA3, a rat IgM antibody (Developmental Studies Hybridoma Bank, Catalog # MC-631), undiluted; anti-SSEA4, a mouse IgG3 antibody (Developmental Studies Hybridoma Bank, Catalog # MC-813-70), undiluted; anti-Tra-1-60 (a gift from Peter Andrews), undiluted; and anti-Tra-1-81, (a gift from Peter Andrews), undiluted. The cells were then washed in wash buffer (PBS) 3 times for 5 minutes each. The remainder of the immunostaining protocol was performed as described in Example 6.

Results

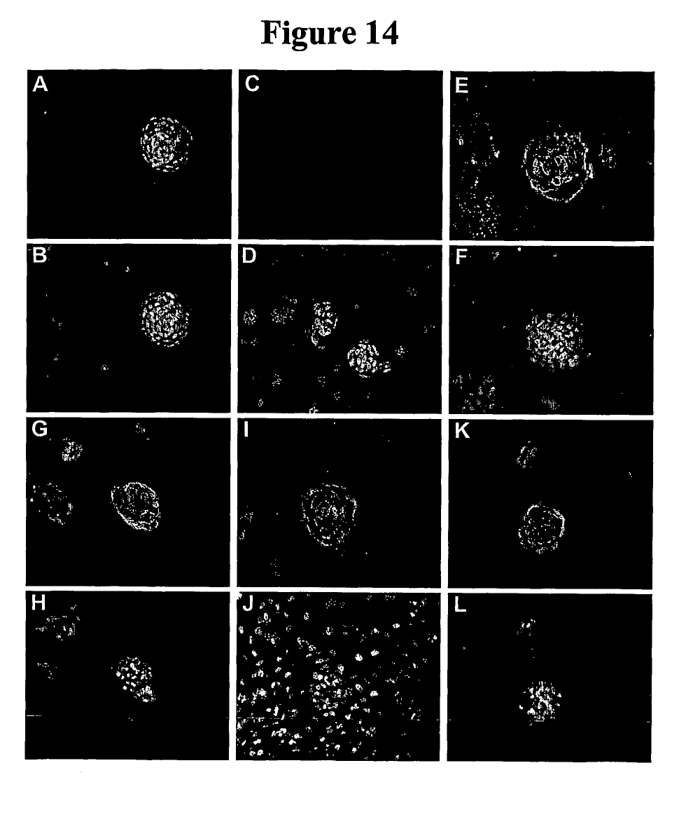

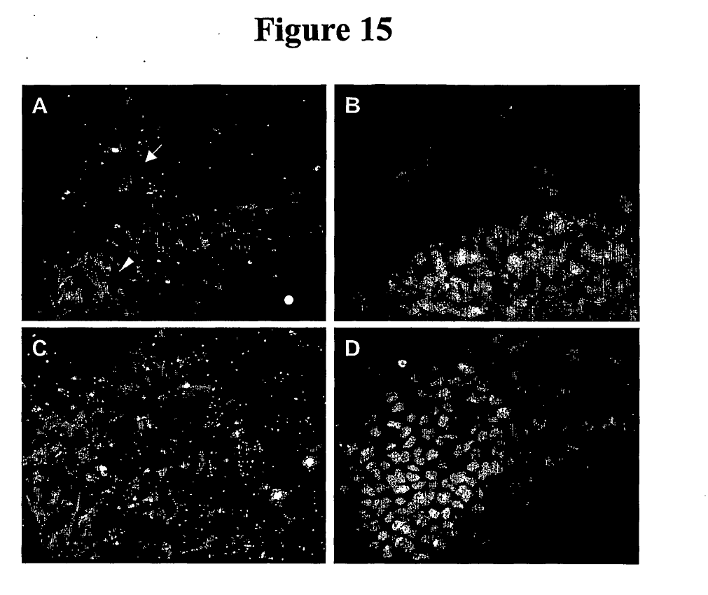

Sorted HESCs contained the expected pattern of marker expression for undifferentiated pluripotent cells: SSEA4⁺, Oct4⁺, Tra-1-60⁺, Tra-1-81⁺, SSEA3⁺, and SSEA1⁻ (FIG. 14). Unexpectedly, SSEA4 selected HESC also expressed the neural progenitor marker nestin (FIG. 15). Manually passaged HESC cultures are typically heterogeneous, demonstrated by colonies that contained a ring of cells expressing nestin that surrounded the bulk of the colony which did not exhibit nestin expression (FIGS. 15A, and 15B). In comparison, SSEA4 selected HESCs showed uniform nestin expression (FIGS. 15C, and 15D). Nestin is a intermediate filament protein that has a distinct pattern in neural progenitor cells. Nestin staining in SSEA4 selected HESCs was organized into a uniformly distributed filamentous staining. The lack of nestin expression in the bulk of manually passaged HESCs in contrast to the uniform nestin staining in SSEA4 selected HESCs indicated that this bulk passaged population, while identical to manually passaged HESCs with regard to expression of markers of pluripotency, could be a downstream cell population with some pre-neural stem cell gene expression characteristics. However, nestin may not be a tightly restricted neural progenitor marker (not shown, and see Kachinsky et al., 1994 Dev. Biol., 165(1):216-28; Wroblewski et al., 1996 Ann. N Y Acad. Sci. 8(785):353-5; Wroblewski et al., 1997 Differentiation, 61(3):151-9; and Mokry and Nemecek 1998, Acta Medica, 41(2):73-80).

### Example 12

Differentiation of SSEA4 Selected HESCs

To test their neural differentiation capacity, SSEA4 selected HESCs were differentiated in essentially serum free conditions as embryoid bodies.

Methods

Essentially serum free embryoid bodies were generated from bulk passaged monolayer HESC colonies as described in Example 6, with or without MEDII conditioned medium.

Cultures were treated with or without 10 μM S18 from day 13 to day 17. After S18 treatment, serum free embryoid bodies were washed several times and cultured further until day 18. Serum free embryoid bodies were cut into pieces to seed down to polyornithine/laminin coated slides. The explants were cultured on slides for 5 days prior to fixation at day 23 for immunostaining.

Essentially serum free embryoid bodies were derived from manually passaged cells, or protease passaged cells. EBs derived from protease passaged cells were formed in the presence of 50% MEDII conditioned medium. The embryoid bodies were exposed to 10 μM S18 in the same medium from day 6 to 9 after derivation. At day 9 the S18 treated sfEBMs matched control sfEBMs not exposed to S18, and matched control manually passaged sfEBs were sectioned to enable the precise determination of the proportion of the total healthy nuclei of an sfEBM that were rosette cell nuclei. The serum free embryoid bodies were embedded using the Immuno-Bed kit (Polysciences, Inc.). Serum free embryoid bodies were rinsed with 1×PBS and fixed in 4% paraformaldehyde, 4% sucrose in 1×PBS for 30 minutes at 4° C. The cells were then washed in 1×PBS and stored at 4° C. PBS was removed and the embryoid bodies were dehydrated by incubation in a series of 25%, 50%, 75% Ethanol/PBS for 5 minutes at room temperature, followed by 100% Ethanol. Infiltration solution was made by adding 0.25 g Benzoyl Peroxide to 20 ml Immuno-Bed Solution A. The ethanol was removed from the serum free embryoid bodies and 1 ml infiltration solution was added. After one hour, the infiltration solution was changed for three 20 min incubations. For embedding, 1 ml solution B (accelerator) was added to 25 ml fresh infiltration solution. The infiltration solution was removed from the serum free embryoid bodies and 0.5 ml embedding solution was added. The samples were transferred to a mold, a block holder was added and the mold was placed at 4° C. to set. 3 micron sections were cut using a Leica microtome, and were stained with DAPI.

Results

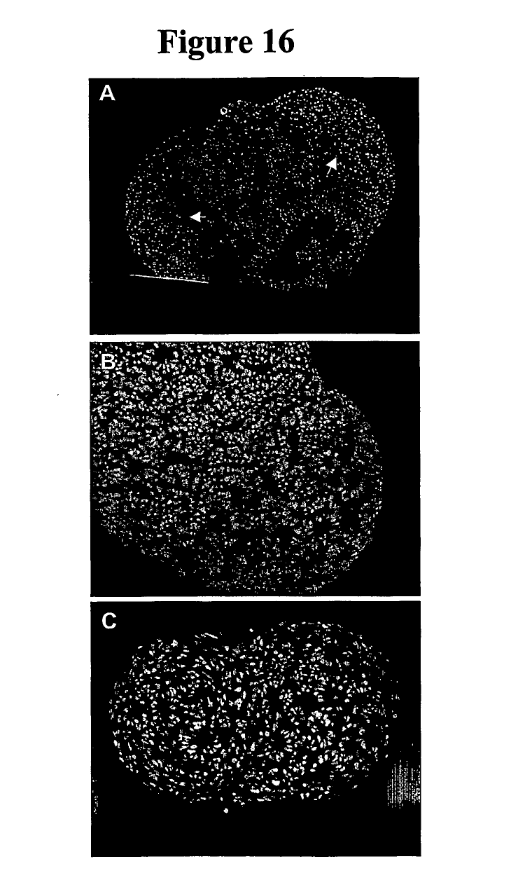

Unlike serum free embryoid bodies derived from HESC crater cells, bulk passaged sfEBMs did not form obvious neural rosette structures in suspension. Sectioning demonstrated that this was because there was a higher proportion of rosette cells formed in a much more uniform distribution in sfEBM derived from bulk passaged SSEA4 selected HESCs (FIG. 16). In suspension, neural rosette structures were therefore obscured in these embryoid bodies because the rosettes were typically smaller and evenly distributed throughout the sfEBMs. In addition, in further experiments, typical large folds of neurectoderm were found in differentiations from bulk passaged and SSEA4 selected cells.

sfEBMs derived from crater cells contained regions of cells with small round nuclei that could not survive within the embryoid body beyond approximately 5 cell widths from the edge of the embryoid bodies. DAPI staining of sections revealed that this cell type was not viable when further in from the edge than 5 cell widths, and the crater cell derived EBs showed significant regions of necrotic or apoptotic nuclei (FIG. 16A). In contrast, sfEBMs derived from SSEA4 selected HESC did not contain obvious regions of this non-rosette cell type, nor did they contain regions of necrotic/apoptotic nuclei in the center of the EB (FIG. 16B). This result further indicated the increased purity of the neural rosette population in sfEBMs derived from SSEA4 selected HESCs. Furthermore, when sfEBM derived from SSEA4 selected HESCs were exposed to 10 μM S18, DAPI staining of sectioned sfEBM indicated that the only surviving cell types had an arrangement and nuclear morphology consistent with a highly enriched population of neural rosette cells (FIG. 16C).

sfEBMs derived from SSEA4 selected bulk passaged HESCs showed significant improvements compared to sfEBMs derived from crater cells when seeded onto polyornithine/laminin coated slides and allowed to differentiate. While sfEBMs derived from crater cells contained some TH+ cells, these TH+ cells did not comprise a large proportion of the culture (i.e., <5% of the neurons were TH+), or form extensive networks, which indicated sporadic DA differentiation in these cultures. sfEBMs derived from SSEA4 selected bulk passaged cells contained extensive networks of TH+ neurons (i.e., >80% of the neurons were TH+).

### Example 13

MEDII Enhanced Differentiation of SSEA4 Selected ES Cells

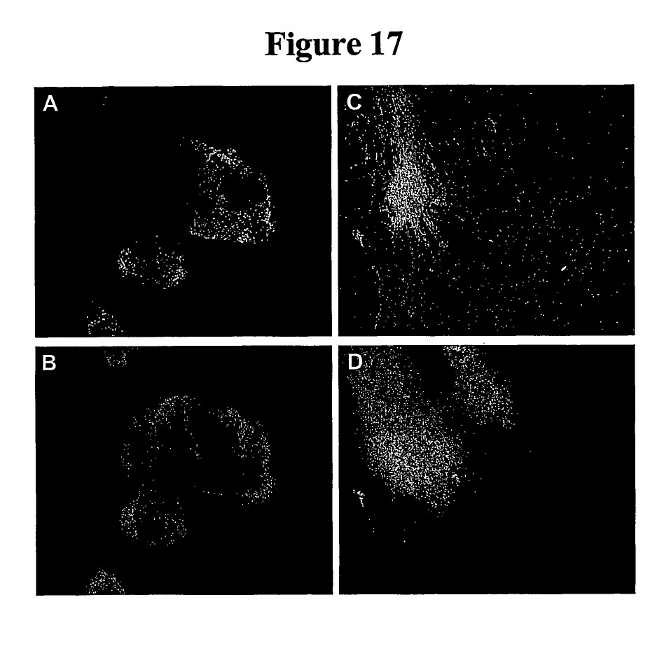

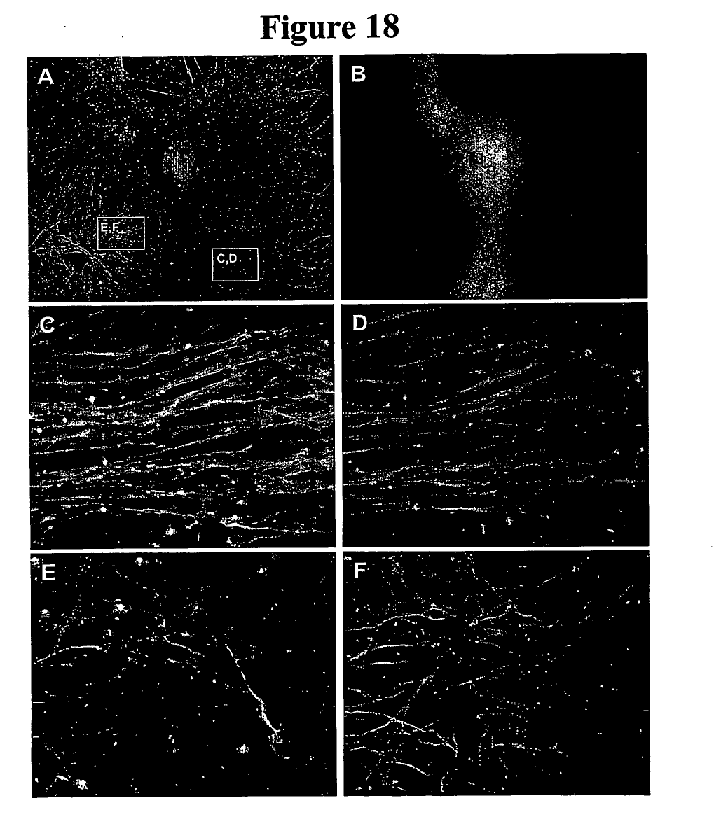

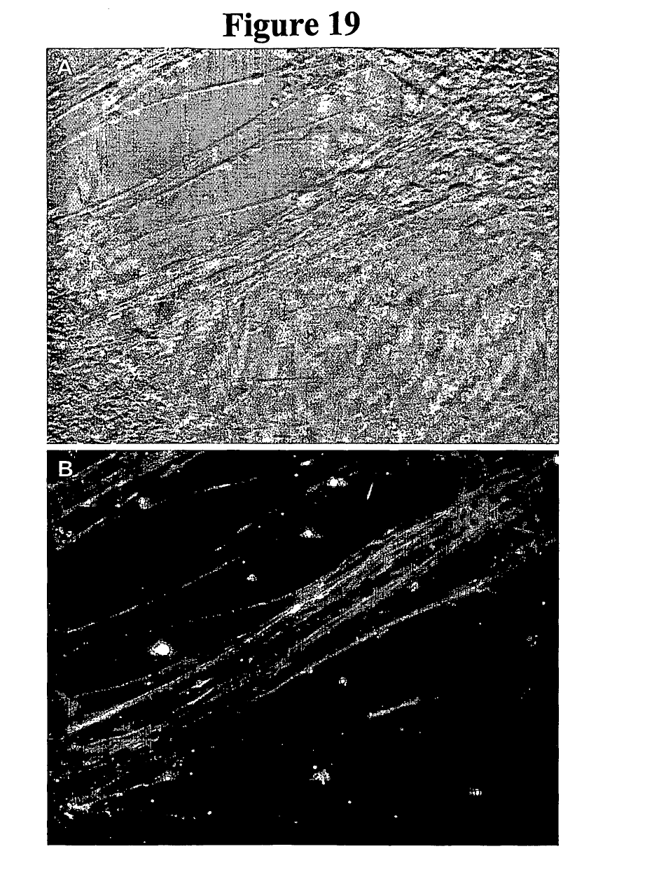

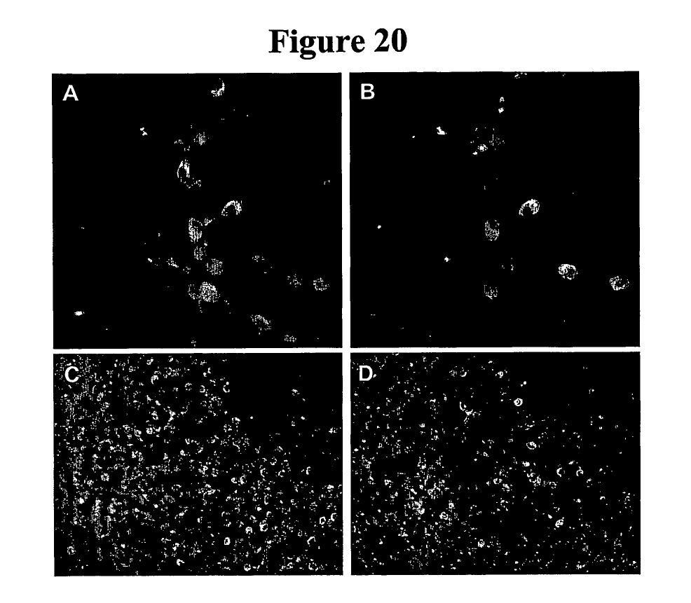

The application of 50% MEDII to embryoid bodies derived from SSEA4 selected bulk passaged cells improved the neural differentiation significantly (FIG. 17). Without MEDII, extensive TH+ networks were present, but the proportion of the culture that did not contain neurons and was presumably a non-neural background cell type varied between approximately 30 and 90%. In the presence of MEDII, a consistently high proportion of the culture contained TH+ neurons, with the background of non-neural regions that was negative for the neuronal marker βIII-Tubulin typically lower than 10%. It was not determined whether the effect of MEDII induced more efficient neuralization or inhibited the generation of non-neural cell types. Furthermore, neurons growing in the presence of MEDII exhibited much longer cellular extensions and they appeared more developed and differentiated than neurons in cultures exposed to FGF2 alone. Under this differentiation scheme, a very high proportion of all neurons, greater than 90%, expressed Tyrosine Hydroxylase (TH), the rate limiting enzyme in dopamine biosynthesis and the standard marker for dopaminergic differentiation. This proportion was determined by analysis of double staining of neural extensions for βIII-Tubulin and TH (FIG. 18), and overlaying Hoffman images with TH immunofluorescence (FIG. 19). The increase in the proportion of TH+ neurons in MEDII treated differentiations appeared to be due to the overall increase in neuronal differentiation, rather than an effect on the proportion of neurons that were dopaminergic, because the proportions of neurons that were TH+ in differentiations not exposed to MEDII was equally high. Another marker of DA cells, VMAT, was expressed in similarly high proportions of cells within the sfEBM cultures. TH+/VMAT−, TH−/VMAT+ and TH+/VMAT+ cells were observed (FIG. 20), possibly indicating temporal variability in the induction of expression of these markers prior to being co-expressed.

### Example 14

Dopamine Release Assays Using sfEBM Cultures

Methods

Dopamine released by depolarized neural cultures was detected by using a Catecholamine-Enzyme Immunoassay (Labor Diagnostika Nord), a clinical diagnostic kit for determination of Dopamine in Plasma and Urine, according to the manufacturer's instructions. The experimental sample was comprised of sfEBMs that had been derived, seeded to polyornithine/laminin coated slides at day 25 and cultured to day 30. Cells were depolarised by exposure to 300 μl 56 mM KCl in minimal MEM (Gibco) per well, for 15 minutes. The medium was removed and frozen.

The dopamine assay was performed as follows: (A) Dopamine was first extracted from the sample using a cis-diol-specific affinity gel, followed by acylation to N-acyldopamine. The supplied standards and 300 μl test sample were pipetted into wells of the cis-diol-specific affinity gel coated plate. 50 μl assay buffer containing 1 M HCl was added to the wells, followed by 50 μl extraction buffer. The plate was covered and incubated for 30 minutes at RT on an orbital shaker (600 rpm). The liquid was decanted, 1 ml wash solution added and the plate was shaken for 5 minutes at 600 rpm. The liquid was decanted and the wash repeated. 150 μl acylation buffer, then 25 μl acylation reagent was added to the wells, followed by shaking at RT for 15 minutes at 600 rpm. The liquid was decanted and 1 ml wash solution added to wells, followed by shaking for 10 minutes at RT at 600 rpm. The liquid was decanted and 150 μl 0.025 M HCl was added to wells to elute N-acyldopamine. 20 μl of the supernatant was used for the determination of dopamine. (3) The N-acyldopamine was converted enzymatically to N-acyl-3-methoxytyamine followed by a competitive Dopamine-EIA. Acylated dopamine in suspension competes with dopamine attached to the solid phase of a microtiter plate for a limited number of antiserum anti-dopamine binding sites until equilibrium is reached. Free antigen and antibody complexes are removed by washing, and antibody complexed with the solid phase dopamine is detected using a secondary antibody conjugated with peroxidase, using TMB as a substrate and detected at 450 nm. The amount of antibody bound to the solid phase is inversely proportional to the dopamine concentration of the sample.

The enzyme solution, catechol-O-methlytransferase, was made no longer than 15 minutes prior to use, and was prepared by reconstitution with 1 ml distilled water, followed by adding 0.3 ml Coenzyme, S-adenosly-L-methionine, and 0.7 ml Enzyme buffer. 25 μl of the enzyme solution was pipetted to assay wells, followed by 125 μl of 0.025 M HCl into the wells for the standards and controls. 10 μl of the extracted standards, controls, two supplied patient urine samples and 125 μl of the extracted sfEBM sample was added to the appropriate wells followed by incubation at 37° C. for 30 minutes. 50 μl anti-dopamine antiserum was added to all wells and shaken at RT for 2 hours at 400 rpm. The wells were aspirated and washed twice with 300 μl wash buffer per well. 100 μl secondary antibody enzyme conjugate was added to the wells and shaken for 30 minutes at RT at 400 rpm. The wells were aspirated and washed 3 times. 100 μl substrate was added to each well and shaken for 35 minutes at RT at 400 rpm in the dark. 100 μl stop solution was added to each well and the absorbance a 450 nm was read within 10 minutes. The absorbance for each standard, control and sfEBM sample were normalized for dilution and were plotted with the linear absorbance of the standards along the y-axis versus log of the standard concentrations in pg/ml along the x-axis.

Results

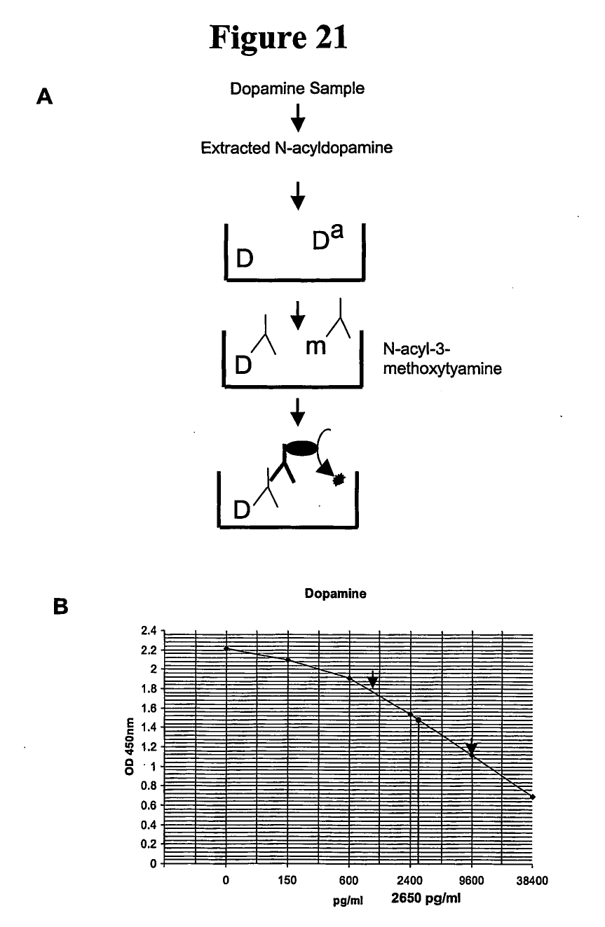

sfEBM cultures were tested for the production and release of dopamine in response to KCl, a depolarizing agent. Cultures were treated with 56 μM KCl for 15 minutes and the culture supernatant assayed for the presence of dopamine using a specific competitive ELISA. A seeded sfEBM culture supernatant contained approximately 2657 pg/ml dopamine after depolarization (FIG. 21B), indicating that dopamine was synthesized by cells within the culture and released when treated with KCl. This value does not indicate the absolute level of dopamine produced, as dopamine levels would be affected by the number of dopaminergic cells seeded as embryoid bodies, their relative level of differentiation with regard to dopamine biosynthetic pathways and vesicle production, and the volume and subsequent dilution of the KCl supernatant. However, this value was similar to the 600 pg/ml found for cultures containing mouse DA neurons (Kim et al., 2002 Nature 418: 50-56), and it also fell between two unknown control samples supplied with the kit, although these values are not directly comparable due to the above reasons.

In addition, dopamine has been detected by HPLC (data not shown).

### Example 15

S18 Treatment of SSEA4 Selected ES Cells

Serum free embryoid bodies and embryoid bodies exposed to 50% MEDII treated with or without S18 were used in the differentiations of SSEA4 selected HESCs. No gross morphological or immunocytochemical staining differences were observed between sfEB/S18− and sfEB/S18+ cultures, or sfEBM/S18− and sfEBM/S18+cultures. This indicated that exposure to S18 induced apoptosis in the possible residual pluripotent cells without otherwise affecting the differentiations.

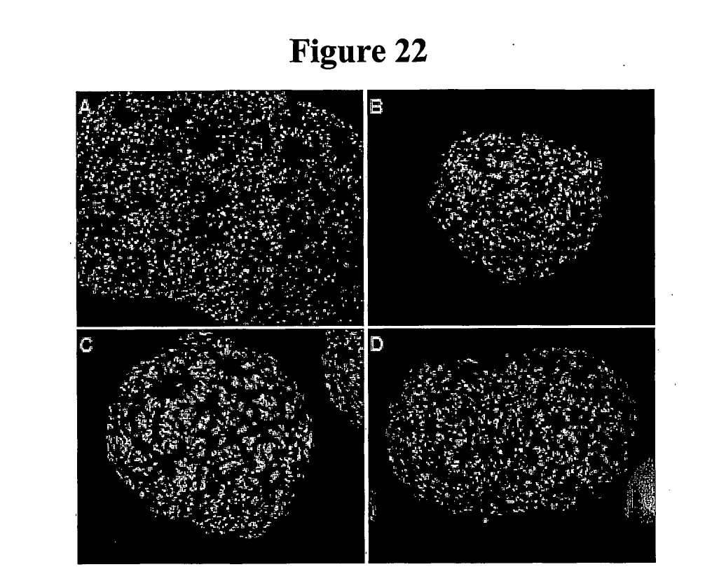

sfEBMs derived from protease passaged cells exposed to 10 μM S18 from day 6 to 9 after derivation were analyzed. In sections of control (untreated) sfEBMs, greater than 80% of the nuclei in the embryoid bodies were associated with rosettes (FIG. 22A). The rosette nuclei were generally elongated, in contrast to regions of smaller round nuclei that were not organized into rosettes. DAPI stained sections of S18 treated sfEBMs showed marked differences from the control sections (FIGS. 22B-D). The overall proportion of nuclei per measured area of sfEBM may have been reduced, but was generally still high. However, nearly all nuclei in the treated sfEBM were elongated in appearance, and rosette structures were still clearly present. The small round nuclei of the presumptively non-rosette cells were very rarely noted. This indicated that a very pure population of neural precursor rosette cells had survived the incubation with S18.

Efficient neural differentiation to predominantly DA neurons that produced and released dopamine was observed in cultures that had been exposed to S18. This high proportion of DA differentiation was significant because it was accomplished in the absence of exogenous inducing signals (MEDII influenced proportion of total neurons, and did not appear to influence proportion of neurons that were TH+) and with a simplified differentiation protocol. sfEBMs derived from SSEA4 selected HESCs that were seeded after 10 days of suspension culture generated neurons, but a low proportion of these were TH+ (data not shown). It is likely that the extended suspension culture described here, around 3 weeks, was a significant contributing factor in the efficient DA differentiation observed.

While the SSEA4 selected HESC expressed the pattern of pluripotent cells that indicate they are an undifferentiated cell population, the uniform expression of nestin may indicate pre-neural stem cell or primitive neural stem cell gene expression characteristics. This may be a contributing factor to the efficient and uniform differentiation of these HESC to neuronal cultures in response to MEDII. It is currently unclear if the protease passaging technique allows the selective growth of this cell type, or if the putative upstream pluripotent cell type in the center of undifferentiated manually passaged HESC does not survive protease passaging.

### Example 16

Differentiation of SSEA4 Selected HESCs in the Presence of Proline

To test their neural differentiation capacity in the presence of proline, SSEA4 selected HESCs were differentiated in essentially serum free conditions as embryoid bodies.

Methods

Essentially serum free embryoid bodies were generated from bulk passaged monolayer HESC colonies as described in Example 6, in the presence of 4 ng/ml FGF2 and 100 μM Proline, or in 4 ng/ml FGF2 with MEDII conditioned medium as a positive control.

Serum free embryoid bodies were cultured in suspension for 17 days, and were cut into pieces and seeded onto polyornithine/laminin coated slides at day 10 or 17. The explants were cultured on slides for 5 days prior to fixation at day 15 or 22, for immunostaining with anti-βIII-Tubulin and anti-Tyrosine Hydroxylase antibodies.

Results

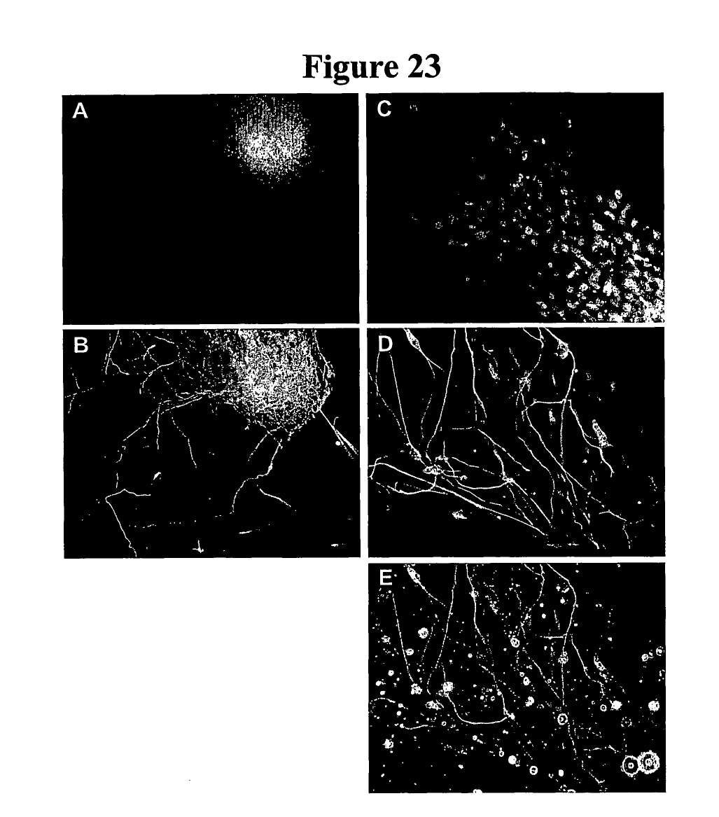

Serum free embryoid bodies grown in FGF2 and 100 μM proline (sfEBP) differentiated to neurons as observed by morphological and immunofluorescent staining of seeded pieces (FIG. 23). Dense networks of βIII-Tubulin+ cells were observed in the majority of seeded pieces (FIGS. 23A, and 23B). A proportion of seeded EB pieces, less than 30%, did not exhibit large networks of βIII-Tubulin+ cells and could represent undifferentiated neural precursors, other neural cell types, or non-neural cells. Double immunofluorescent staining indicated that greater than 90% of the neurons generated were dopaminergic, co-expressing βIII-Tubulin and TH (FIGS. 23C, D, and E). This level of dopaminergic differentiation was consistent with that observed with bulk passaged SSEA4 selected HESCs differentiated in the presence of FGF2/MEDII. Unlike sfEBMs, sfEBPs did not flatten when pieces were seeded, and generally remained in a more globular structure. As noted previously, sfEBMs exhibit large outgrowths of a monolayer cell type(s), which neurons and neural extensions grew on top of. Therefore, sfEBM cultures exhibited long neuron extensions radiating from seeded pieces, which was not as pronounced in sfEBP pieces. Therefore the effect of proline on the neural differentiation was pronounced, but did not mimic all the effects of MEDII. However, it is not clear if the proliferation of the monolayer cell type(s) will be beneficial for cell transplantations, and could effectively lower the proportions of neurons within the total culture, despite it being beneficial for in vitro differentiation of neural processes.

### Example 17

Differentiation of SSEA4 Selected HESCs in Differing Media Formulations

To test their neural differentiation capacity in the presence of different media formulations, SSEA4 selected HESCs were differentiated in essentially serum free conditions as embryoid bodies.

Methods

Essentially serum free embryoid bodies were generated from bulk passaged monolayer HESC colonies as described in Example 6, in the following media formulations:

| | | βIII-Tubulin | TH positive |
|---|---|---|---|
| | Media Formulation | positive cells | cells |
| A | minimal medium (DMEM, | Not | Not |
| | N2, L-Glutamine, | determined | determined |
| | Penicillin, Streptomycin) | | |
| B | minimal medium with | 24% | Not |
| | 4 ng/ml FGF2 | | determined |
| C | minimal medium with | 73% | 51% |
| | 100 μM Proline | | |
| D | minimal medium with | 63% | 60% |
| | 200 μM Proline | | |
| E | minimal medium with | 31% | 58% |
| | 100 μM Proline and | | |
| | 4 ng/ml FGF2 | | |
| F | minimal medium with | 36% | 37% |
| | 200 μM Proline and | | |
| | 4 ng/ml FGF2 | | |
| G | DMEM, F12, N2, L- | 50% | 52% |
| | Glutamine, Penicillin, | | |
| | Streptomycin and | | |
| | 4 ng/ml FGF2 | | |
| H | DMEM, F12, N2, L- | 25% | 32% |
| | Glutamine, Penicillin, | | |
| | Streptomycin, 4 | | |
| | ng/ml FGF2 and 50% MEDII | | |

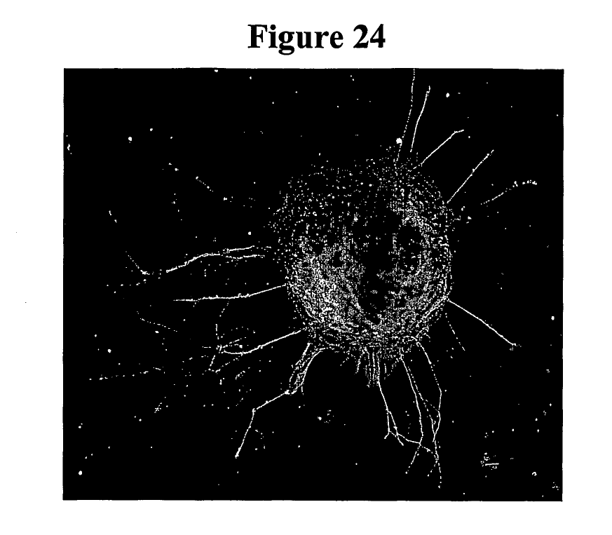

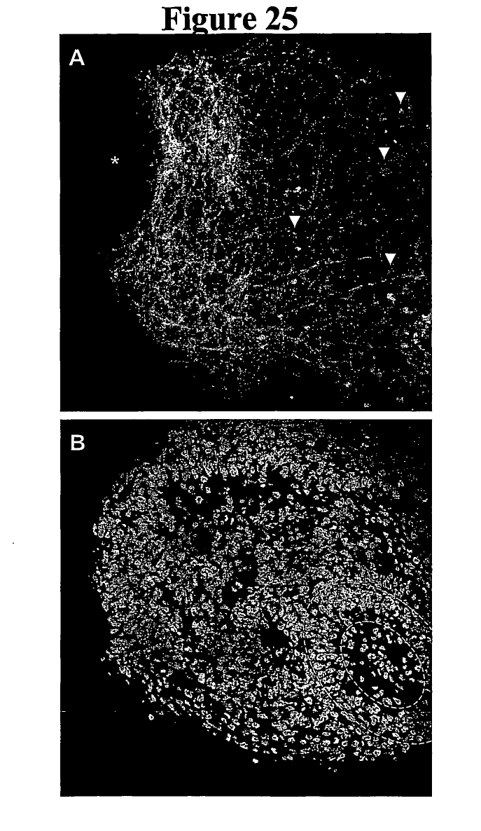

Serum free embryoid bodies were cultured in suspension for 3 weeks. Morphological differences were apparent between the cultures. Low proliferation in minimal medium (A) was observed, as well as increased cell death, with an external layer of cell death surrounding what appeared to be a viable and proliferative core of cells. Minimal medium with proline (C, D) seemed to exhibit a higher proliferation or survival rate, although still contained increased cell death compared to FGF2 containing conditions (B, E-H). Conditions B-H showed good proliferation over the course of the experiment. Serum free embryoid bodies were cultured in suspension, and were cut into pieces, seeded onto polyornithine/laminin coated slides at day 21 and fixed at day 25. Immunostaining with anti-βIII-Tubulin demonstrated the presence of extensive networks of neurons in all conditions, even in minimal medium (Condition A) that contained no FGF2, Proline, F12, or MEDII (FIG. 24). This was indicative that this differentiation protocol utilizes an intrinsic neural differentiation capacity of HESC, rather than exogenous neural inducing factors.

Cytospins of disaggregated serum free embryoid bodies were performed at day 21 to enable the counting of the proportion of βIII-Tubulin or TH positive cells generated in the different media formulations. III-Tubulin is a marker for differentiating neurons, but also known to be expressed in HESC colonies, although this expression is not neuronal-like (Carpenter et al., Exp. Neurol. 172, 383-397). Expression of βIII-Tubulin in seeded serum free embryoid bodies (FIGS. 23B, D; and FIG. 24), and in whole mount stainings of sfEBPs in suspension (FIG. 25A), was only observed in cells of overt neuronal morphology. Therefore, using this marker to count the proportion of neurons in sfEBPs is not expected to be influenced by the potential persistence of pluripotent cells. The immunostaining of these cytospins with an anti-TH antibody did not generate as strong a signal, and was therefore not likely to be as accurate as the βIII-Tubulin count

To count proportions of neurons in serum free embryoid bodies, cytospins were immunostained with anti-βIII-Tubulin (Sigma, #T8660) or mouse anti-TH monoclonal antibodies (PeIFreez Biologicals, #P80101-0), detected with alexa-488 conjugated anti-mouse secondary antibody and nuclei were stained with DAPI. Two color fluorescent images were taken under 10× magnification and merged, and double positive signals were scored as neuronal cell bodies, or TH+ neuronal cell bodies against the total nuclei count. A minimum of three randomly sampled fields and 250 or 100 nuclei for βIII-Tubulin or TH, respectively, were counted for each condition. The highest proportion of βIII-Tubulin positive cells was observed in L-Proline conditions (Conditions C and D), indicating the purest population of neurons generated in this comparison. The relatively lower proportion of neurons observed in FGF2/MEDII conditions (Condition H, 25%) indicated the overgrowth of the presumptive glial or glial progenitor monolayer cell type observed morphologically, rather than a reduced total number of neurons. The presence of a lower proportion of neurons in any condition containing FGF2 (Conditions B, E-H) presumably reflected the known activity of this factor in maintaining undifferentiated neural progenitors (Okabe et al., Mech Dev. 1996: 59(1):89-102).

This data indicated that neuronal differentiation occurred in suspension, and sfEBPs in particular were likely to be a mix of neural precursors and differentiating neurons. L-Proline media (Conditions C and D) appeared to exhibit the purest population of neurons, at more than 50% of the cells in a sfEBP, but it was not determined if these cells were as differentiated as observed previously in seeded sfEBM, where there are non-neuronal cell types for neurites to grow on. Where analyzed, immunostaining of cytospins with anti-TH also revealed similar proportion of TH+ neurons in each condition as total neurons, given the caveat of the lower confidence of the accuracy of the count. Regardless, counting of TH+ cell bodies indicated that the large majority of neurons in all the conditions tested were TH+. It is likely that this analysis will be improved as the cytospin immunostain assay for TH is optimized further. An example of this would be to develop a triple stain assay for TH/βIII-Tubulin/DAPI.

The differentiation of βIII-Tubulin positive neurons in all the conditions, including minimal, chemically defined medium (Condition A), indicated that this system was based on the intrinsic capacity of HESC to differentiate to neurons, rather than the addition of exogenous “neural inducing” factors. In this scenario, the activities of L-proline, FGF2 and MEDII could be related to the proliferation and survival of cell types generated intrinsically within the system. Alternatively, components of the N2 supplement (insulin, transferrin, progesterone, selenite and putrescine) could effect a neural inducing activity. However, these components, apart for transferrin, were tested and shown to not play a significant role in neural specification in a monolayer system of mouse ES cell differentiation (Ying et al., 2003 Nat. Biotech. 21:183-186).

### Example 18

Differentiation of SSEA4 Selected HESCs in Various Concentrations of L-Proline

To test their neural differentiation capacity in the presence of a range of L-Proline concentrations, SSEA4 selected HESCs were differentiated in essentially serum free conditions as embryoid bodies.

Methods

Essentially serum free embryoid bodies were generated from bulk passaged monolayer HESC colonies as described in Example 6, in the presence of the media set out below.

| | Media Formulations |
|---|---|
| A | Minimal medium (DMEM, N2, L-Glutamine, Penicillin, |
| | Streptomycin) |
| B | Minimal medium with 5 μM Proline |
| C | Minimal medium with 50 μM Proline |
| D | Minimal medium with 100 μM Proline |
| E | Minimal medium with 500 μM Proline |

Essentially serum free embryoid bodies formed in the presence of proline containing medium are termed sfEBPs. sfEBPs were cultured in suspension for three weeks, and were passaged by manual cutting at around the 2 week mark. sfEBPs exhibited a high level of cell death throughout the first 3 weeks of suspension culture, with an outer layer of dead cells and generally slow proliferation when compared to EB formation in FGF2/MEDII conditions in previous experiments. At around 3 weeks, sfEBPs exhibiting low cell death and distinct neural rosette structures/folds were observed in all conditions. The appearance of this type of sfEBP was noticeably enhanced in the 50 μM Proline condition. A higher proportion of the sfEBPs exhibited this morphology in the 50 μM Proline condition than in other conditions, and their morphology was superior, with fewer associated dead cells and more noticeable neural rosette structures.

sfEBPs derived in 50 μM L-proline have been passaged and maintained in a proliferative state in suspension culture for more than 7 weeks after initial derivation and 34 weeks after proliferation of neural rosette structures. This indicates that under these conditions there is a balance between rosette proliferation and neuronal differentiation. When seeded to polyornithine/laminin, a high proportion of DA differentiation was still exhibited. When seeded in 50 μM L-Proline, a high degree of cell death was observed in outgrowths, although good networks of βIII-Tubulin+ neurons were still viable. When seeded in FGF2/MEDII medium, morphologically healthy outgrowths were observed to contain neurons and cells similar to the presumed glial or glial progenitor derived from rosettes. This indicated that there were cell types within the sfEBPs that were continuously generated that could not survive in the minimal conditions. It is likely that this indicated that these cells were differentiated from rosette cells.

sfEBPs grown in 50 μM L-proline were fixed in suspension and immuonstained with anti-βIII-Tubulin or DAPI in a wholemount assay. These sfEBPs were mounted and optically sectioned using a Leica TCS SP2 Spectral Confocal Microscope. Networks of βIII-Tubulin+ neurons were visualized throughout the sfEBP, as were DAPI stained neural rosettes (FIGS. 25A and B).

The high degree of cell death observed over the first 3 weeks is likely to be indicative of the continual generation of cell types that are not viable under these serum- and serum replacer-free conditions, until the generation, maturation, or adaptation of a neural rosette cell that can proliferate in minimal medium, which is enhanced in the presence of L-proline.

### Example 19

Abnormal Karyotype in Bulk Passaged HESCs

The karyotypes of cells passaged using protease passaging and SSEA4 selection were examined. Karyotypes of exemplary cell lines are shown below. P#/# indicates the total number of passages, followed by the number of passages after SSEA4 selection. For example P42113/9-42 total passages, with SSEA4 selection at passages 33 and 29. Prior to SSEA4 selection, the cell lines were passaged with collagenase/trypsin for several passages. Therefore the protease passaged cell lines were initially passaged using manual passaging methods, then passaged with collagenase/trypsin, were SSEA4 selected, and then continued to be passaged with collagenase/trypsin. The cells passaged using manual passaging only all demonstrated normal karyotypes. Cells passaged using collagenase/trypsin protease passaging had abnormal karyotypes. At least 7 lines passaged with collagenase/trypsin had abnormal karyotypes. The most common abnormalities were trisomy 12 and 17, although trisomy 1, 7, 8 and 14 were also noted.

Protease passaging was initiated several passages prior to SSEA4 selection. It is notable that BG01 cell line passaged 35 times using manual passaging had a normal karyotype, while the same cell line that was passaged 32 times, with more than 20 of those passages using collagenase/trypsin, demonstrated an abnormal karyotype with 19 of 20 metaphases being abnormal. Passaging cell cultures with collagenase/typsin and dissociating them to an essentially single cell culture appears to lead to the development of an abnormal karyotype.

| Passaging | Cell | | | Metaphase | |
|---|---|---|---|---|---|
| Method | Line | Passage | Karyotype | counted | Comments |
| Manual | BG01 | p35 | 46, XY | 10 | Normal |
| Passage | BG02 | p19 | 46, XY | 20 | Normal |
| | BG03 | p17 | 46, XX | 30 | Normal |
| Protease | BG01 | p42/13/9 | 47, XY, +17 | 26 | Triploid 17 |
| Passage | | | 46, XY | 4 | |
| | BG01 | p32/20 | 47, XY, +17 | 12 | Mixed |
| | | | 48, XY, +12, +17 | 3 | karyotype |
| | | | 49, XY, +1, +12, +17 | 4 | with |
| | | | 46, XY | 1 | triplodies |
| | BG01 | p84/17 | 50, XXY, +12, +14, +17 | 40 | Mixed |
| | | | 51, XXY, +7, +12, +14, +17 | 2 | karyotype |
| | | | 51, XXY, +8, +12, +14, +17 | 2 | with |
| | | | | | triplodies |

We claim:

1. A human pluripotent embryonic stem cell culture, wherein the cells of the culture do not express SSEA1, express SSEA3, SSEA4, Oct4, Tra-1-60, Tra-1-80, and express nestin substantially uniformly.

2. The cell culture of claim 1, wherein the cell culture was dissociated to an essentially single cell culture.

3. The cell culture of claim 2, wherein a majority of the cells have an abnormal karyotype.

4. The cell culture of claim 3, wherein the abnormal karyotype comprises a trisomy of at least one autosomal chromosome.

5. The cell culture of claim 4, wherein the autosomal chromosome is selected from the group consisting of chromosomes 1, 7, 8, 12, 14, and 17.

6. The cell culture of claim 5, wherein the autosomal chromosome is chromosome 12 or 17.

7. The cell culture of claim 3, wherein the abnormal karyotype comprises a trisomy of more than one autosomal chromosome.

8. The cell culture of claim 7, wherein the autosomal chromosome is selected from the group consisting of chromosomes 1, 7, 8, 12, 14, and 17.

9. The cell culture of claim 8, wherein the autosomal chromosome is chromosome 12 or 17.

10. A method of culturing a human pluripotent embryonic stem cell comprising,
a) selecting a human pluripotent cell using an anti-SSEA4 antibody; and
b) maintaining a culture of the cell by passaging the cell using a protease treatment, wherein the cells of the culture do not express SSEA1, express SSEA3, SSEA4, Oct4, Tra-1-60, Tra-1-80, and express nestin substantially uniformly.

11. The method of claim 10, wherein the protease treatment comprises the sequential use of Collagenase and trypsin.

12. The method of claim 10, wherein the cell is maintained by using a protease treatment for at least 13 passages.

13. The method of claim 10, wherein a majority of the cells of the culture have an abnormal karyotype.

14. The cell culture of claim 13, wherein the abnormal karyotype comprises a trisomy of at least one autosomal chromosome.

15. The cell culture of claim 14, wherein the autosomal chromosome is selected from the group consisting of chromosomes 1, 7, 8, 12, 14, and 17.

16. The cell culture of claim 13, wherein the abnormal karyotype comprises a trisomy of more than one autosomal chromosome.

17. The cell culture of claim 16, wherein the autosomal chromosome is selected from the group consisting of chromosomes 1, 7, 8, 12, 14, and 17.

18. The method of claim 11, wherein Collagenase is used at a concentration of approximately 1 mg/ml for approximately 5 minutes, and wherein trypsin is used at a concentration of approximately 0.05% for approximately 30 seconds.

19. A method of providing a human cell culture enriched in neural cells, comprising forming an embryoid body comprising the human pluripotent embryonic stem cell of claim 10.

20. The method of claim 19, wherein the embryoid body is formed by culturing the cell with an essentially serum free medium.

21. The method of claim 20, wherein the essentially serum free medium is a MEDII conditioned medium.

22. The method of claim 21, wherein the MEDII conditioned medium is a Hep G2 conditioned medium.

23. The method of claim 21, wherein the MEDII conditioned medium comprises one or more proline residues or a polypeptide containing proline residues.

24. The method of claim 23, wherein the MEDII conditioned medium comprises proline at a concentration of approximately 50 μM.

25. The method of claim 19, wherein the embryoid body is formed by culturing the cell with a minimal medium.

26. The method of claim 25, wherein the minimal medium is essentially proline free.

27. The method of claim 25, wherein the minimal medium comprises one or more proline residues, or a polypeptide containing proline residues.

28. The method of claim 0, wherein the minimal medium comprises proline at a concentration from approximately 50 μM to approximately 250 μM.

29. The method of claim 25, wherein the minimal medium is essentially FGF free.

30. The method of claim 25, wherein the minimal medium is essentially MEDII free.

31. A human pluripotent cell produced by the method of claim 10.

32. A human cell culture enriched in neural cells, produced by any the method of any one of claims 19-30.

33. The human cell culture of claim 32, wherein greater than approximately 80% of the human cell culture comprises neural cells.

34. The human cell culture of claim 33, wherein greater than approximately 90% of the neural cells express tyrosine hydroxylase.

35. A method for treating a patient, comprising a step of administering to the patient having a neural disease a therapeutically effective amount of the human cell culture enriched in neural cells of claim 32.

36. The method of claim 35, wherein the neural disease is Parkinson's disease.

37. A method of culturing a human pluripotent embryonic stem cell comprising,
a) providing a human pluripotent embryonic stem cell culture;
b) passaging the cell culture using a protease treatment to thereby disperse the cell to an essentially single cell culture; and
c) culturing the essentially single cell culture in the presence of a feeder cell, a conditioned medium, or a minimal medium to thereby culture the human pluripotent embryonic stem cell.

38. The method of claim 37, wherein the protease treatment comprises the sequential use of Collagenase and trypsin.

39. The method of claim 38, wherein Collagenase is used at a concentration of approximately 1 mg/ml for approximately 5 minutes, and wherein trypsin is used at a concentration of approximately 0.05% for approximately 30 seconds.

40. The method of claim 37, wherein the feeder cell is a freshly plated feeder cell.

41. The method of claim 40, wherein the feeder cell is a mouse embryonic fibroblast.

42. The method of claim 40, wherein the feeder cell has been plated for less than 10 hours.

43. The method of claim 40, wherein the feeder cell has been plated for less than 6 hours.

44. The method of claim 40, wherein the feeder cell has been plated for less than 2 hours.

45. A human pluripotent embryonic stem cell culture produced by the method of claim 37, wherein the cells of the culture do not express SSEA1, express SSEA3, SSEA4, Oct4, Tra-1-60, Tra-1-80; and express nestin substantially uniformly.

46. The human pluripotent cell embryonic stem culture of claim 45, wherein a majority of the cells of the culture have an abnormal karyotype.

47. The human pluripotent embryonic stem cell culture of claim 46, wherein the abnormal karyotype comprises a trisomy of at least one autosomal chromosome.

48. The human pluripotent embryonic stem cell culture of claim 47, wherein the autosomal chromosome is selected from the group consisting of chromosomes 1, 7, 8, 12, 14, and 17.

49. The human pluripotent embryonic stem cell culture of claim 46, wherein the abnormal karyotype comprises a trisomy of more than one autosomal chromosome.

50. The human pluripotent embryonic stem cell culture of claim 49, wherein the autosomal chromosome is selected from the group consisting of chromosomes 1, 7, 8, 12, 14, and 17.

51. A method of producing a human pluripotent embryonic stem cell culture enriched in neural cells comprising,
a) providing a human pluripotent embryonic stem cell culture;
b) passaging the cell culture using a protease treatment to thereby disperse the cell culture to an essentially single cell culture;
c) culturing the essentially single cell culture in the presence of a feeder cell, a conditioned medium, or a minimal medium; and
d) forming an embryoid body comprising the essentially single cell culture by culturing the cell culture with an essentially serum free medium,
to thereby produce the human cell culture enriched in neural cells.

52. The method of claim 51, wherein protease treatment comprises the sequential use of Collagenase and trypsin.

53. The method of claim 52, wherein Collagenase is used at a concentration of approximately 1 mg/ml for approximately 5 minutes, and wherein trypsin is used at a concentration of approximately 0.05% for approximately 30 seconds.

54. The method of claim 51, wherein the essentially serum free medium is a MEDII conditioned medium.

55. The method of claim 54, wherein the MEDII conditioned medium is a Hep G2 conditioned medium.

56. The method of claim 54, wherein the MEDII conditioned medium comprises one or more proline residues or a polypeptide containing proline residues.

57. The method of claim 56, wherein the MEDII conditioned medium comprises proline at a concentration of approximately 50 μM.

58. The method of claim 51, wherein the feeder cell is a freshly plated feeder cell.

59. The method of claim 58, wherein the feeder cell is a mouse embryonic fibroblast.

60. The method of claim 58, wherein the feeder cell has been plated for less than 10 hours.

61. The method of claim 58, wherein the feeder cell has been plated for less than 6 hours.

62. The method of claim 58, wherein the feeder cell has been plated for less than 2 hours.

63. The method of claim 51, wherein the minimal medium comprises one or more proline residues, or a polypeptide containing proline residues.

64. The method of claim 63, wherein the minimal medium comprises proline at a concentration from approximately 50 μM to approximately 250 μM.

65. The method of claim 51, wherein the minimal medium is essentially proline free.

66. The method of claim 51, wherein the minimal medium is essentially FGF free.

67. The method of claim 51, wherein the minimal medium is essentially MEDII free.

68. A human cell culture enriched in neural cells produced by the method of claim 51.

69. A method for treating a patient, comprising a step of administering to the patient having a neural disease a therapeutically effective amount of the neural cell of claim 68.

70. The method of claim 69, wherein the neural disease is Parkinson's disease.

71. The human cell culture of claim 68, wherein greater than approximately 80% of the human cell culture comprises neural cells.

72. The human cell culture of claim 71, wherein greater than approximately 90% of the neural cells express tyrosine hydroxylase.

73. A method for treating a patient, comprising a step of administering to the patient having a neural disease a therapeutically effective amount of the human cell culture enriched in neural cells of claim 68.

74. The method of claim 73, wherein the neural disease is Parkinson's disease.


SEQUENCE LISTING (5 sequences)

SEQ ID NO: 1 - protein, 4 aa, Artificial Sequence
  Description of Artificial Sequence Synthetic polypeptide
GPRP

SEQ ID NO: 2 - protein, 4 aa, Artificial Sequence
  Description of Artificial Sequence Synthetic polypeptide
GPGG

SEQ ID NO: 3 - protein, 4 aa, Artificial Sequence
  Description of Artificial Sequence Synthetic polypeptide
VAPG

SEQ ID NO: 4 - protein, 4 aa, Artificial Sequence
  Description of Artificial Sequence Synthetic polypeptide
RPKP

SEQ ID NO: 5 - protein, 11 aa, Artificial Sequence
  Description of Artificial Sequence Synthetic polypeptide
RPKPQQFFGLM